(19) United States
(12) Patent Application Publication
(10) Pub. No.: US 2025/0186319 A1
(43) Pub. Date: Jun. 12, 2025

(54) ANAPLEROTIC COMPOSITIONS AND USES THEREOF

(71) Applicant: EXCELLA BIOSCIENTIFIC LTD, Nahariya (IL)

(72) Inventor: Sabina GLOZMAN, Nahariya (IL)

(21) Appl. No.: 19/037,761

(22) Filed: Jan. 27, 2025

Related U.S. Application Data
(63) Continuation-in-part of application No. 18/417,470, filed on Jan. 19, 2024.
Application No. 18/417,470 is a continuation-in-part of application No. PCT/IL2022/050777, filed on Jul. 19, 2022.
(60) Provisional application No. 63/265,310, filed on Dec. 13, 2021.
(60) Provisional application No. 63/223,322, filed on Jul. 19, 2021.
(60) Provisional application No. 63/624,902, filed on Jan. 25, 2024.

(51) Int. Cl. A61K 8/37; A61Q 17/04

(52) U.S. Cl. CPC A61K 8/37; A61Q 17/04; A61K 2800/31; A61K 2800/522; A61K 2800/805

(57) ABSTRACT

Anaplerotic Olebiome compositions comprising a blend of anaplerotic MOCL esters; a Triolein-enriched plant carrier oil, and at least one lipophilic antioxidant and/or antiaging ingredient are provided.

### CROSS-REFERENCE TO RELATED APPLICATIONS

This application is a continuation-in-part of U.S. application Ser. No. 18/417,470 filed Jan. 19, 2024, which is a continuation-in-part of PCT Application No. PCT/IL2022/050777 filed Jul. 19, 2022 which, in turn, claims the benefit of U.S. provisional application Ser. No. 63/265,310 filed Dec. 13, 2021 and U.S. provisional application Ser. No. 63/223,322 filed Jul. 19, 2021. This application also claims the benefit of U.S. provisional application 63/624,902 filed Jan. 25, 2024. The disclosures of all of these applications are hereby incorporated in their entirety by reference herein.

### FIELD OF THE INVENTION

The present invention relates to products derived from naturally occurring sources of unsaturated fatty acids, anaplerotic compositions, manufacturing processes, and applications in pharmaceuticals, cosmetic and food industries. Specifically, the present invention relates to products and anaplerotic compositions promoting longevity, treating inflammatory conditions, SPF-boosting, and maintaining or restoring microbiome balance.

### BACKGROUND OF THE INVENTION

Anaplerosis refers to a cyclical enzymatic process that generates catalytic intermediates of the TCA (tricarboxylic acid) cycle, which carry acetyl-CoA. Within mitochondria, these intermediates are enzymatically oxidized using a specific pool of nutritional anaplerotic substrates. This process replenishes the metabolite pool of the TCA cycle.

To maintain homeostasis, anaplerosis works in tandem with cataplerosis, which removes excess TCA intermediates as organic byproducts. These intermediates are then metabolized in the cytosol through processes like gluconeogenesis, sugar storage, and lipid accumulation. Anaplerotic substrates include propionate, lactate, pyruvate and its precursors (e.g., PEP [phosphoenolpyruvate] and pentanoate), derivatives of propionyl-CoA (e.g., methylmalonyl-CoA and succinyl-CoA), odd-chain fatty acids (e.g., 3-hydroxyvalerate), certain amino acids (alanine, aspartate, glutamine, glutamate, tyrosine, phenylalanine), branched-chain amino acids (valine, leucine, isoleucine, beta-amino-isobutyric acid), and C5 ketone bodies.

Anaplerosis can be induced by hypoxic conditions and operates through various pathways, such as transaminations between aspartate and pyruvate, the purine nucleotide cycle, oxidative deamination of glutamate, and the carboxylation of pyruvate. Additionally, TCA cycle metabolites, such as succinic acid (SA), citric acid (CA), and their derivatives, act as anaplerotic modifiers. These metabolites have been clinically shown to function as secretagogues with protective applications. Recent research also indicates that TCA cycle intermediates influence cell reprogramming, working synergistically with Yamanaka factors. As an essential regenerative mechanism, anaplerosis plays a critical role in replenishing these intermediates, supporting cell rejuvenation and lifespan extension. Odd-chain fatty acids, in particular, have unique utility in enhancing cellular functionality and longevity.

Lipids are fundamental not only as structural components of cellular membranes but also as key contributors to mitochondrial energy production, biogenesis, cell signaling, and organelle function. They serve as anaplerotic substrates, replenishing TCA cycle intermediates and restoring metabolic balance. Lipid droplets (LDs), once thought to be mere storage organelles, have recently been identified as significant regulators of the Integrated Stress Response (ISR), alongside stress granules (SG). Research by Labbé et al. (2024) highlights LDs' role in central carbon metabolism and lipid droplet biogenesis under stress.

Aging is commonly associated with genomic instability, telomere attrition, epigenetic alterations, loss of proteostasis, deregulated nutrient sensing, mitochondrial dysfunction, cellular senescence, stem cell exhaustion, and altered intercellular communication. Recent studies underscore the growing importance of lipidome homeostasis in aging. For instance, research by Sadowski et al. demonstrated a significant negative correlation between age and the total lipid content in female skin. Triglycerides (TGs), which constitute approximately 80% of sebum lipids between the ages of 20 and 40, were shown to decrease markedly with age. By ages 50-55, both the absolute and relative amounts of sebaceous TGs had declined by 25%, with a further 50% reduction observed in older age groups.

Another manifestation of aging is the senescence of the sebaceous skin microbiome, which includes the face, chest, and back. This microbiome is relatively simple, consisting predominantly of species such as Cutibacterium, Staphylococcus, Pseudomonas, Candida, and Malassezia yeast. As sebaceous glands (SG) undergo senescence with age, they experience atrophy, reduced sebum-lipid secretion, and changes in sebum composition. These functional changes manifest as skin dryness, diminished brightness, and a decline in the skin microbiome's diversity. Importantly, imbalances in sebum secretion contribute to disruptions in the equilibrium of the skin microbiome. Anhydrous, lipophilic lipid-based formulations are ideal for delivering a variety of lipophilic anti-aging ingredients. These formulations provide inherent antimicrobial stability, eliminating the need for harmful preservatives. Moreover, they are highly effective in maintaining the skin's hydrophobic barrier, preventing moisture loss and reducing epidermal water loss-key contributors to skin aging. This is especially important when natural sebum production declines, such as in postmenopausal women or within internal lipid membranes. Additionally, these formulations improve the intracellular absorption of lipophilic agents by seamlessly fusing with and integrating into hydrophobic cellular membranes.

Oil-in-water and water-in-oil emulsions, as well as liposomes, are also widely used as delivery systems for lipophilic compounds. However, these systems can be costly, and liposomes have specific limitations, such as vulnerability to phagocytosis and low encapsulation efficiency of active ingredients. Vegetable oils are often comedogenic, greasy, and heavy for topical application, presenting challenges for industrial formulation and practical use. For example, olive oil-recognized for its longevity benefits and containing 50-80% triolein, can cause serious irritation in sensitive skin.

Non-edible oils also pose challenges due to safety concerns, making them less suitable for use in delivery systems. Similarly, mineral oils, once widely employed as delivery systems and lubricants in anhydrous formulations, have seen reduced usage due to safety risks associated with polycyclic aromatic hydrocarbons (PAHs), including potential carcinogenic and genotoxic effects.

Specialty lipids such as medium-chain triglycerides (MCTs) (C10 and C12) and very long odd-chain fatty acids (VLOCFAs) (C15 and C17) have been recognized in peer-reviewed research as effective anti-aging compounds, primarily for their ability to enhance mitochondrial metabolism-a key hallmark of longevity, Dunn et al. (2023) on the benefits of MCTs in aging and neurodegenerative diseases (Front. Aging Neurosci., 15:1230467. doi: 10.3389/fnagi.2023.1230467) and Pfeffer and Jaudszus (2016) on the multifaceted roles of odd-chain fatty acids (Adv Nutr., 7 (4): 730-4. doi: 10.3945/an.115.011387).

MCTs serve as a source of ketogenic energy for mitochondria and are widely utilized in metabolic and anti-aging products, which represent a multibillion-dollar industry trend. However, challenges remain for formulating MCTs in a sensorily appealing manner. These challenges include their dominant coconut-like scent, temperature-dependent solidification, greasiness, and ethical concerns related to palm oil sourcing. Addressing these issues is essential to optimize MCT-based products.

VLOCFAs are emerging as industrially significant longevity-focused lipids, primarily derived in small quantities from dairy fats. Despite their potential, VLOCFAs are prone to oxidation due to their double bonds, necessitating high levels of preservatives, additives, and antioxidants. Furthermore, their thick, greasy, and viscous properties pose significant challenges for large-scale industrial formulation.

Both MCTs and VLOCFAs can be incorporated into anhydrous delivery systems at limited concentrations to address compliance and industrial scalability challenges. They hold potential for use in topical formulations and cosmeceutical products, however, optimizing their properties for industrial applications remains a significant challenge.

There has been a long-standing and unmet need to develop lipid-based anhydrous delivery systems that utilize alternatives to mineral oils and to provide new sources for regenerative anaplerotic substances that may be additive and synergistic to multiple known anti-ageing life span protecting factors. These alternatives should replicate the desirable properties of mineral oils, as similar sensory characteristics, such absorption rates, transparency, and textures, while providing “light” and fast-absorbing oil qualities.

### SUMMARY OF THE INVENTION

It is a principle object of the present invention to provide efficatious, stable, user firendly, cost-effective, lipid-based anhydrous delivery systems enriched with anaplerotic substrates.

It is another object of the present invention to provide multifunctional compositions enriched with anaplerotic substrates useful in attenuating aging of the skin and reparing microbiome disbalance, named Olebiome.

It is another object of the present invention to provide Olebiome compositions enriched with anaplerotic substrates useful as SPF boosters.

It is another object of the present invention to provide effective and safe cosmetic, food and therapeutics for various skin abnormalities associated with inflammation.

It is another object of the present invention to utilize neutral lipids compositions comprising combination of glycerol and esters of MOCL, and balanced with Triolein enriched edible plant oils serving a as precursor of TriAza derivatives, in multifunctional anaplerotic Olebiome composition as LD mimetic and replacement approach.

It is another object of the present invention to utilize neutral lipids compositions comprising combination of glycerol and esters of MOCL, and balanced with Triolein enriched edible plant oils serving as a of TriAza precursor derivatives, in multifunctional anaplerotic composition to balance mitochondrial, epigenetic, and other hallmarks of aging.

It is another object of the present invention to utilize neutral lipids Olebiome compositions comprising combination of glycerol and esters of MOCL, and balanced with Triolein enriched edible plant oils serving as a precursor of TriAza derivatives, in multifunctional anaplerotic composition as longevity booster.

It is another object of the present invention to provide a blend of Medium Odd Chain Lipids (MOCL) with edible plant carrier oils enriched with Triolein.

It is another object of the present invention to provide advantageous Olebiome anaplerotic compositions of a blend of MOCL with edible vegetable carrier oils enriched with Triolein. Triolein can be, without limitation, a precursor of MOCL exposed by partial oxidation under quality-controlled conditions (e.g. QC specification such as peroxide value), and can be characterized by having a prolonged shelf-life and non-prone to further oxidation, comprising C9 monocarboxylic and dicarboxylic (azelaic) fatty acids and antioxidants as main ingredients.

It is another object of the present invention to provide advantageous anaplerotic compositions characterized by having a prolonged shelf-life and non-prone to oxidation, comprising MOCL end products of oxidation cleavage of triolein characterized by the lack of unsaturated double bonds and oxidation stable thereby obtaining stability (under accepted QC specifications) of the drug unstable against oxidation as well as being applicable for commercial production in a large scale.

It is another object of the present invention to provide advantageous anaplerotic compositions comprising MOCL, such as C9 monocarboxylic (pelargonic) and/or dicarboxylic (azelaic) fatty acids and precursors of thereof in glycerol and non-glycerol esters form to avoid unneccessary safety risks, such asirritation as prven dermatologically (also on sensitive skin) and of free fatty acids form that known to casue irritation in some indivisuals, especially sensitive skin subjects, when applied topically.

It is another object of the present invention to provide topical anaplerotic compositions based on C9 MOCLMOCL and derivatives thereof for the treatment of skin abnormalities associated with inflammation, microbiome disbalance and/or aging.

It is another object of the present invention to provide an anaplerotic food grade composition comprising Medium Odd Chain Lipids (MOCL) or derivatives thereof, wherein said MOCL comprising esterified linear, branched or glycerol esterified fatty acids.

It is another object of the present invention to provide topical anaplerotic compositions based on MOCL and derivatives thereof for the treatment of skin abnormalities associated with aterations in sebaceous microbiome.

It is another object of the present invention to provide topical anaplerotic compositions based on C9 MOCL and derivatives thereof for the treatment of skin abnormalities associated mitochondrial dysfunction.

It is another object of the present invention to provide topical anaplerotic compositions based on C9 MOCL and derivatives thereof for preventing skin abnormalities associated impaired lipogenesis and mitochondrial dysfunction.

It is another object of the present invention to provide topical anaplerotic Triglycerides (TG) lipids-based and fatty acids (FA) precoursors enriched compositions for preventing and/or improving and/or trating and/or repairing impaired lipidome and mitichondrial dysfuntion.

It is another object of the present invention to provide anaplerotic topical compositions comprising Triolein in carrier oil and a combination of Medium Odd Chain Tryglicerydes (MOCT) with MOCL.

It is another object of the present invention to provide a sebum replacement therapy based on active anaplerotuc compositions.

It is another object of the present invention to provide a manufacturing processes for TriAza.

It is another object of the present invention to provide a manufacturing process for TriAza from vegetable, marine or microbial source, or by chemical synthesis through oxidative cleavage of C9 carbon unsaturated bond of unsaturated fatty acids chains in triglycerides, without hydrolysis under acidic conditions. Such TriAza as a naturally derivative can be used in food for shift towards anaplerotic regeneration of TCA produced bioenergy.

It is another object of the present invention to provide a functional compositons based on a prodrug conjugate hydrolysable by a lipase specific to hydrolysis of Triglycerides.

It is another object of the present invention to provide a co-formulation of both safe nonanoic acids derivatives as multifunctional composition with diverse functionalities, preferably with an additional “functional enhancer” targeted to specific function in selected for certain utilization/application.

It is another object of the present invention to provide an Triazelaine, anaplerotic composition comprising wherein Triazelaine is obtained from a vegetable source.

It is another object of the present invention to provide a process for the preparation of the composition according to the above embodiments, comprising oxidating triolein of the olive oil without hydrolyzing the acyl glycerol bond.

It is another object of the present invention to provide a method of balancing the microbiome of the gut in a subject in need, comprising administering to the subject one or more of the above anaplerotic compositions.

It is another object of the present invention to provide a method of balancing the microbiome of the skin in a subject in need, comprising administering to the subject one or more of the above anaplerotic compositions.

It is another object of the present invention to provide s a product for consumption comprising and/or prepared from one or more of the above anaplerotic compositions.

It is another object of the present invention to provide a food grade anaplerotic compositions comprising Medium Odd Chain Lipids (MOCL).

It is another object of the present invention to provide a food grade anaplerotic composition comprising at least one active biomolecule.

It is another object of the present invention to provide a food grade anaplerotic composition comprising triazelaine.

It is another object of the present invention to provide an anaplerotic composition comprising one or more odd medium chain fatty acid (MOCL) or a derivative thereof, and optionally at least one functional enhancer.

It is another object of the present invention to provide quality control for production process to avoid generation of lipid hydroperoxides and push process towards TriAZa precursors generation.

It is another object of the present invention to provide multifunctional formulation comprising neutral lipids as lipid droplets (LDs) mimicking composition, enriched with triolein, MOCL.

It is another object of the present invention to provide multifunctional formulation comprising neutral lipids as lipid droplets (LDs) mimicking composition, enriched with triolein, MOCL characterized by superior photooxidation protection SPF booster effect.

It is another object of the present invention to provide multifunctional formulation comprising neutral lipids as lipid droplets (LDs) mimicking composition, enriched with triolein, MOCL characterized by for health span longevity boosting effect.

It is another object of the present invention to provide multifunctional formulation comprising neutral lipids as lipid droplets (LDs) mimicking composition, enriched with triolein, MOCL characterized by immediate skin barrier protection effect within 4 hours after first administration.

It is another object of the present invention to provide multifunctional formulation comprising neutral lipids as lipid droplets (LDs) mimicking composition, enriched with triolein, MOCL characterized by mitochondrial ATP booster effect.

It is another object of the present invention to provide multifunctional formulation comprising neutral lipids as lipid droplets (LDs) mimicking composition, enriched with triolein, MOCL characterized by microbiome balancing effect.

It is another object of the present invention to provide Olebiome, a unique delivery of lipophilic antiaging actives.

It is another object of the present invention to provide a method for improving a metabolic fat-related condition in a subject in need, comprising administering to the subject one or more of the above anaplerotic compositions.

It is another object of the present invention to provide the use of the above anaplerotic compositions and lipophilic delivery systems, as a medicament.

It is another object of the present invention to offer longevity functionalities comparable to or exceeding those of MCTs and VLOCFA.

It is another object of the present invention to provide compositions mirroring the beneficial properties of mineral oil, including serving as a delivery system for lipophilic anti-aging actives, providing lubrication, enhancing sensory experience and physical texture, while ensuring safety and compatibility with sensitive skin.

It is another object of the present invention to provide a product for consumption comprising and/or prepared from the composition according to the above embodiments.

### BRIEF DESCRIPTION OF THE DRAWINGS

FIG. 1 demonstrates MOCL as postbiotics metabolized/hydrolyzed by dose-dependent lipase-mediated activity in-vitro (purified lipase of Pseudomonas strain bacteria used in this experiment), measured by nonanoic acid derivatives-Pelargonic and Azelaic acid from the representative TriAza partially oxidized (by ozonolysis) batch named L-PABS (-Lipase-Pathogen Activated Biological Substrate, i.e. prebiotic This proves that L-PABS/TriAza −Triolein-partially oxidized Triolein- as prebiotic substrate microbial lipase and hydrolyzed by microbial enzymes-nonanoic acids are postbiotics)). At maximal dose of lipase MOCL postbiotics-Azelaic acid and Pelargonic acid were at maximal as qualified by optical visual spot and the density of TriAza/L-PABS spot reduced;

FIG. 2 demonstrates P. acnes-associated lipase-mediated activity of time-dependent release (day 1 and day 5 in the culture), of MOCL Azelaic acids-postbiotic with maximum lipase hydrolysis on day 5 from the representative batches (L-PABS-Lipase, Pathogen Activated Biological Substrate, i.e. prebiotic-acting as prebiotic substrate for P.acnes) under incubation with live culture of Corynobacterium acnes (P. acnes) in comparison to non-hydrolyzed L-PABS-TriAza batch without exposure to bacteria enzyme in culture medium and MOCL standards of azelaic and pelargonic acids. L-PABS post-ozonation batch is partially oxidized batch still comprise impurities of pelargonic acid without P.acnes culture (−), Pelargonic acid release is evident by optical visually increased spot at day 5 indicates on the presence of nonanoic precursors-Triolein impurities, and/or partially oxidized TriAza precursors (optionally 000-ozonides, present in L-PABS prebiotic that epoxides or peroxides), hydrolyzed with P.acnes enzymatic activity and release MOCL postbiotic-Pelargonic acid-Pel.;

FIG. 3 demonstrates lipase-mediated specificity to hydrolysis by Triglyceride Lipase (Lipase) and release of Azelaic acid comparison of batches (L-PABS, i.e. prebiotic, TriAza) obtained by oxidative ozonolysis and by chemical synthesis specificity of lipase-mediated hydrolysis of Azelaic acid by Triglyceride Lipase and non-sensitivity/specificity to esterase-E treatment (FIG. 3A); and dose dependent sensitivity and selectivity of Azelaic acid release by Triglyceride Lipase nonarmed visual spot increase of Azelaic acid release form TriAza −L-PABS batch at 50 mcM of bacterial lipase, but not by Phospholipase at the same concentration, from representative TriAza batches (L-PABS-Lipase-Pathogen Activated Biological Substrate, i.e. prebiotic) (FIG. 3B);

FIG. 4 demonstrates HPLC-SEC chromatogram of representative Olebiome composition of TriAza “green” chemistry produced batch that comprise some MOCL esters end products of oxidation reaction, such as Poly-Aza-Polymers (poly-azelaic glycerol), Mono-Aza-MG-monoglycerol of azelaic acid; Di-Aza-DG diglycerol of azelaic acid; TriAza −TG-triglycerol/triglyceride of azelaic acid and Pelargonic-monocarboxylic nonanoic acid.

FIG. 5 demonstrates HPLC-SEC follow-up of L24-0003 (FIG. 5A) and L24-0007 (FIG. 5B). picture of the reaction set-up of L24-0007 (FIG. 5C) and HPLC-SEC trace obtained during the “green” synthesis of L24-0007 (TriAza batch), final composition demonstrating kinetics of formation of Poly-Aza, Tri-Aza, Di-Aza, Mono-Aza, and azelaic acid (FIG. 5D);

FIG. 6 demonstrates HPLC-SEC follow-up of L24-0024 (TriAza batch), (FIG. 6A) and L24-0040 (FIG. 6B), and picture of the reaction set-up of L24-0040 (TriAza batch), (FIG. 6C);

FIG. 7 demonstrates HPLC-SEC trace of L24-0024 (TriAza batch), after washing with hot water (FIG. 7A), and HPLC-SEC trace of collected AZA from the washing aqueous phases (FIG. 7B), and picture of a comparison between the obtained products (FIG. 7C);

FIG. 8 demonstrates GC chromatograms obtained for epoxidized VHOSO starting material (FIGS. 8A and 8B), L24-0073 (TriAza batch), (FIG. 8C) and L24-0115 (TriAza batch), (FIGS. 8D and 8E). The products were either analyzed after cleavage of the fatty acid moieties from the triglyceride by methylation (FIGS. 8A, 8C, 8D) or after silylation of the oil prior to injection in the GC equipment (FIGS. 8B and 8E);

FIG. 9 demonstrates a picture of the reaction set-up for L24-0117 (TriAza batch);

FIG. 10 demonstrates composition in Free Fatty Acids, MG, DG and TG from L24-0135 (TriAza batch): GC chromatogram (FIG. 10A), and HPLC-SEC trace of L24-0135 (TriAza batch) (FIG. 10B);

FIG. 11 demonstrates composition in Free Fatty Acids, MG, DG and TG from L24-0137 (TriAza batch): GC chromatogram (FIG. 11A) and HPLC-SEC trace (FIG. 11B). In both cases, the analyses of the reaction mixture are shown before and after adding the bleach reagent;

FIG. 12 demonstrates Triolein as prebiotic sufficient as a single nutrient for lipophilic microbiome growth by using colorimetric assay on solid medium: upper picture-emulsified Triolein only on the spot allows bacterial colonization as a sufficient substrate, also known as essential for lipophilic microbiome expressing triglyceride lipase, that are accepted as virulent factor-inducing inflammation if overexpressed-unbalanced). Bottom: negative control-no growth on the agar only (left), no growth on emulsifier only (right);

FIG. 13. Dihydroxylation of epoxidized VHOSO;

FIG. 14 demonstrates representative qualitative and quantitative analytical results characterize relative recovery of C9 TriAza derivatives of ozonolysis reaction including: 000-ozonides, AzaA-(nonanoic dicarboxylic) Azelaic acid, Pelargonic (nonanoic monocarboxylic) acid/PA, TriAza −TG-C9/TG9- and derivatives (Mono-Aza; Di-Aza, etc.);

FIG. 15 demonstrates selection of TLC separation method by analysis of spot distribution in different running solvents post-ozonolysis (000-ozonides, AzaA-(nonanoic dicarboxylic) Azelaic acid, Pelargonic (nonanoic monocarboxylic) acid/PA, TriAza −TG-C9/TG9- and derivatives (Mono-Aza; Di-Aza, etc.), oleic acid, aa/ac acid-acetic acid used as a solvent; and,

FIG. 16 demonstrates screening antibacterial efficacy of MOCL (azelaic-Az. Acid and Pelargonic Acid) and -TriAza (batch VA4-1) with and without emulsifier.

### DETAILED DESCRIPTION OF THE INVENTION

The present invention is now described more fully hereinafter with reference to the accompanying examples and drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

According to some embodiments, the invention provides neutral lipid-based anaplerotic composition.

According to some embodiments, the above composition is Lipid Drop LD-mimetic, meaning composition comprising mostly (over 70%) neutral lipids, such as esters of Fatty acids, essentially including glycerol esters, preferably triglycerides and most preferably triolein, and non-glycerol esters of fatty acids. In specific inventive “Olebiome” composition disclosed herein: said LD mimetic are enriched with Triolein and esters of MOCL.

According to some embodiments, the above composition is designed for alleviating age-related symptoms in human skin and such as SPF boosting effect, skin barrier protection, effective for perfecting the appearance and sensorially of the aged skin. As used herein, the term “perfecting” can be interchangeable with terms such as, without limitation, “improving”, “smoothing”, “feeling younger”, and “representing younger phenotype”, “feels and/or looks differently better than before the application”, and refers, without limitation to, “beautifying the skin”, “glowing skin”, “moisturizing the skin”, “skin whitening”, “wrinkle reduction”. “Perfecting” can also mean that user of said MOCL based composition may experience rapid results (such as improvements in one or more of the above parameters) after use even after single or multiple applications of the composition. “Perfecting” is defined and measured as rapid reduction of TEWL within four hours after use, or subjective description such as “feel so different” positive sensorial feeling, “feel so good” positive sensorial perception, “feel so younger”. According to some embodiments, the above LD mimetic composition is named Olebiome. Olebiome is neutral LD mimetic composition comprising over 50% of neutral lipids, wherein at least 25-50% of said composition Triolein-enriched pure carrier oil, and 50% of said LD mimetic composition are triglycerides, and at least 25-50% are lipid-based anaplerotic MOCL esters blend. According to some embodiments, the above Olebiome composition can be characterized by microbiome balancing mechanism of action, wherein said neutral lipid ingredients, preferably triolein, serve prebiotic for lipophilic skin bacteria and MOCL-nonanoic acid derivatives serves postbiotics that control microbial growth, wherein TriAza (produced by “green” synthesis, or oxidation, or partial oxidation of Triolein), may serve as a prebiotic-Triolein-mimetic, that control microbiome growth, and release azelaic acid as postbiotic by activity of microbial lipase), wherein when TriAza produced by partial oxidation of Triolein-both azelaic acid and pelargonic acids are released by microbial lipase as postbiotic and control microbial growth. Control of microbial growth by TriAza is specific and selective to lipase expressing (lipophilic) microbiome and does not affect other strains.

According to some embodiments, MOCL ingredients of the above Olebiome compositions may serve as precursors of both lipid prebiotics and postbiotic of the microbiome, therefore inducing microbiome balance and even antimicrobial control functions. Nonanoic acid precursors (heptanoic and Triolein at least partially oxidized under controlled conditions) and derivatives that are essential ingredients of Olebiome composition, for example TriAza, tripelargonin, nonoate esters that release pelargonic/nonanoic monocarboxylic or/and dicarboxylic-azelaic acid, by its own activity, as TriAza and its derivatives, or under release of said monocarboxylic acid metabolically, for example under cell esterase hydrolysis, and then resulted nonanoic acids control overgrowth of virulent microbiome.

According to some embodiments, microbial lipase, as an overexpressed virulent factor of lipophilic microbiome, when unbalanced, release lipase that decompose MOCL nonanoic precursor to pelargonic and/or azelaic acids. According to some embodiments, the above compositions comprise “probiotic”, i.e. healthy-protective microbiome, said probiotic are approved for use in topical (oil, wax, balm, soap, oleogel, organogel, stick, emulsion, cream, etc.), dental (tooth paste, tooth rinse, tooth cream, tooth soap), mucosal (intranasal, intravaginal, transmucosal sublingual or buccal), or oral formulations (supplements, additives, or food formulations, gummies), in conjunction with triglyceride-triolein like prebiotics and/or nonanoic postbiotics, and serves as anaplerotic modulators. In some cases, additional non microbiome balancing agents, e.g. microbiome-active derivatives/of probiotics, postbiotic-metabolites of probiotics, and prebiotic-nutritional for probiotics) can be included in the Olebiome formulation (like lipids with microbiome balance effect-medium even chain lipids-caprylic, lauric, and derivatives of thereof, such as ozonated fatty acids, epoxidized fatty acids, hydrogenated fatty acids alcohols, etc. short chain fatty acids-SCFA—e.g. butyric, cholic, succinic, malic, formic, propionic, etc. and SCFA derivatives, such as salts and alcohols-propanol, butanol, etc.; derivatives of thereof, preferably-amphiphilic-glycerol monolaurate, or sugar microbiome-active derivatives, natural or naturally derived polyphenols, flavonoids, terpenes, essential oils, or oligo- or poly-saccharides, e.g. xylose, trehalose, xylitol, alginate, etc.

According to some embodiments, Olebiome composition is neutral lipid, i.e. LD mimetic based anhydrous formulation, e.g. formulated without water/hydrophilic part, as an oil.

According to some embodiments, Olebiome neutral lipids, LD mimetic, anaplerotic composition that induce microbiome balance may be formulated with surfactants and emulsifiers. According to some embodiments, Olebiome is formulated as oil-in-water, water-in oil emulsion, microemulsion, nano-emulsion, or cream. According to some preferred embodiments, said formulated as cream, balm, soap, stick, butter, organogel, oleogel, foam, paste, deodorant, that comprise about 70% oil and lipophilic or amphiphilic ingredients, such as Olebiome MOCL, Triolein enriched lipids, described herein. In additional examples, Olebiome composition may comprise amphiphilic surfactants and emulsifiers, for example green surfactants, e.g. glycerol monolaurate, choline azelate, glucoside fatty acid, etc., solidifying lipids such as sterols and waxes, and rest are hydrophilic ingredients for example sugars, (sugars are oligo and disaccharides) for example trehalose, xylose, ribose, mannose, polysaccharides, such as natural polymers, for example high and low molecular weight hyaluronic acid, alginates, algae derived sugars (oligo or disaccharides) and polysaccharides, medicinal mushrooms sugars and polysaccharides, Ganoderma lucidum polysaccharides (GLPS), derived from the medicinal mushroom Ganoderma lucidum or other medicinal mushrooms. In additional embodiment, Olebiome composition enriched with Triolein and MOCL, also contain about 15-258, not more that 40% of amphiphilic, or/and hydrophilic polymers, such as natural polymers, such as peptides, polypeptides and proteins, such as lipophilic peptides (dipeptides, tripeptides, preferably lipid esters of said peptides, preferably palmitate peptides or cyclic peptides), natural protein polypeptide polymers such as growth factors, extracellular matrix elastin, gelatin, collagen proteins its derivatives and precursors. Said natural or naturally mimetic polymers can be in liquid, solid, semisolid or particulate, micro- or nano-(sub-micro-) particulate form. Said formulations can be oral, topical mucosal (intranasal, dental, or oral transmucosal). More specifically, Olebiome compositions comprise at least 70%:
a) a blend of MOCL esters, enriched with glycerol and non-glycerol esters of nonanoic monocarboxylic and dicarboxylic acids (pelargonic and azelaic and precursors and derivatives of thereof);
b) a Triolein-enriched olive oil or other plant carrier oil enriched with Triolein to, at least about 50%, preferably—about more preferably about and more, and most preferably Triolein-enriched carrier oil is used in Olebiome formulation in “pure” form; and,
c) at least one lipophilic antiaging cosmetic or aesthetic ingredient, for example antioxidant, preferably more.

As used herein, the term “pure Triolein-enriched oil” refers, without limitation, to Triolein enriched plant oil, such as olive oil, apricot oil, argan oil, wherein “Pure” used oil preferably is that raw material for said carrier oils is obtained with QC-quality control documentation, indicating “oxidation” status of this utilized in the Olebiome formulation raw material specifying PV about 3 or less, this utilized “pure” raw material is specified by industrial certificate of analysis (CoA), or external or internal lab raw material qualification, that said provided CoA indicates PV value about 3 or less, and also preferably AV value and most preferably other lipid qualification of said raw material oil, for example fatty acid composition by GC analysis, most preferably pharmaceutical or medical grade carrier oil Said qualification is done by method described herein or measured by kits as established din the state of the art. According to some embodiments of the above compositions, the blend of MOCL esters comprises at least two, more preferably three or more MOCL esters. In one embodiment, at least one ester of the blend is a glycerol ester, and at least one ester is a non-glycerol ester of MOCL. In one embodiment, at least MOCL one ester are heptanoic and nonanoic (medium odd chain C7-C9) fatty acid derivatives. In one embodiment, the total MOCL esters between 25% to 50%, concentration is preferably 30- to 60%, most preferably 35-70% and more.

According to some embodiments of the above compositions the concentration of the Triolein enriched olive oil or other plant carrier oil 25% to 85%; more preferably 25%-50%, more preferably 30%—to 60%, most preferably 35-70%. According to some embodiments of the above compositions the concentration of the Triolein enriched olive oil or other plant carrier oil is 25%, 30%, 35%, 40%, 45%, 50%, 55%, 60%, 65%, 70%, 75%, 80%, and 85%.

According to some embodiments of the above compositions, the at least one lipophilic antioxidant is an anti-aging ingredient. In the context of the invention, the term “anti-aging ingredient” is referred without limitation, to list of accepted in the state of the art antiaging ingredients (https://www.byrdie.com/anti-aging-ingredients-biology;

https://www.webmd.com/beauty/ss/slideshow-anti-aging-ingredients; to antioxidants refers to Free radical-neutralizing ingredients, such as vitamin C, coQ10, vitamin E, ferulic acid, natural polyphenols, such as resveratrol, quercetin, lipoic acid, terpenes, chelators, alkaloids. Examples of antiaging cosmetic antiaging ingredients, such as sunscreens; anti-pigmentation/whitening ingredients-hydroquinone, retinoids, retinoid mimetic such as vitamin A and bakuchiol, vitamin c, kojic acid; anti-sagging ingredients, such as peptides and ceramides; anti-dull ingredients, alpha hydroxy acids and retinoids (like retinol), collagen-inducing and anti-wrinkle ingredients such as hyaluronic acid, polyglutamic acid, exosomes, growth factors, retinol, and again-vitamin C; longevity promoting ingredients. In addition listed in the state of the art longevity supplements can be also included in the list of anti-aging ingredients described in the state of the art (https://insights.avea-life.com/healthy-ageing/supplements/best-longevity-supplements/), mostly are antioxidants too, representing by vitamin B derivatives such as, NMN (nicotinamide mononucleotide), a nucleotide derived from ribose and nicotinamide and other “mitochondrial boosters” as described below; ubiquinol-CoQ10 is a fat-soluble antioxidant compound essential for mitochondrial energy production, extracellular matrix-ECM polymers, such as hyaluronic acid polysaccharide and ECM proteins, such as elastin and collagen, essential minerals and metals (zinc, selenium, iron, chromium, copper), vitamins such as vitamin A, B, E, C, D, K, and plant derived antioxidants, resveratrol, berberine, curcumin, other naturally derived antioxidants such as spermidine, ergothioneine, pyrroloquinoline quinone, fisetin, quercetin, EGCG, glutathione derivatives, such as NAC, caffeic acid, etc.

According to some embodiments, the above compositions are undiluted.

According to some embodiments, the above Olebiome compositions are anhydrous, free of water. As used herein, the term “free of water” refers, without limitation, to a composition anhydrous at least for about 70%, at least for 50%, most preferably more than 60%, about 70% or more, of the Olebiome composition is composition that is lipid based, hydrophobic, lipid-soluble composition, composed from (1) Triolein enriched-carrier oil; (2) C9-nonanoic esters enriched MOCL blend; and (3) antiaging antioxidant ingredients, as described above. The rest of the Olebiome composition may comprise additional lipophilic ingredients, fats and lipids, such as SCFA, sterols, cholesterols, phytosterols, waxes, essential oils, phospholipids, glycolipids, ceramides, squalene, omega 3 fatty acids, cannabinols, liposoluble phenols, polyphenols, resveratrol, rutin, quercetin, cannabinoids, jasmonates, oxylipins, EGCG, flavonoids, alkaloids, terpenes and terpenoids, essential oils, preferably fatty acid derivatives, such as soaps and salts, such as ozonated, hydrogenated lipids, lipid solubilizing solvents, such as based alcohols, LCFA-long chain fatty acids, including omega 3 fatty acids, liposoluble extracts, lipopeptides, pterostilbene, vitamins, etc.). In addition some part, about 15-25%, not more than 30%, of the Olebiome composition can comprise hydrophilic and amphiphilic ingredients that do not include pure water (H₂O), but may comprise non-water natural or naturally based hydrophilic or amphiphilic moieties, and polymers, more preferably biopolymers, such as, sugars, oligosaccharides (xylitol, trehalose), polysaccharides (hyaluronic acid, alginate, etc.), amino acids and derivatives, peptides, polypeptides, proteins and aggregates-complexes of thereof, and polyamines (e.g. spermidine) and indole derivatives.

According to some embodiments, the above compositions further comprise at least one active hydrophobic, hydrophilic or insoluble SPF/sunscreen component and at least one excipient and/or surfactant. The excipient can be single- or multiple-component excipient.

According to some embodiments, the above compositions may be formulated, without limitation, as oil, oil enriched emulsion, microemulsion, spray, soap, balm, foam, cream, in liquid, solid, or semi-solid form, preferably as oleogels, organogels, formulation comprising glycerol monolaurate, selected waxes blend, solid stick with waxes. According to some embodiments, the above formulations are suitable for Olebiome oil base to be co-formulated by state of the formulation art principles with the most types of SPF agents, including hydrophilic, hydrophobic and insoluble, e.g. inorganic, for example coated particles of zinc oxide as a most green SPF for sensitive skin.

According to invention provides an some embodiments, the anaplerotic composition with Triolein containing oils as a precursor for Triazelaine, wherein Triazelaine is obtained from a vegetable source. Triazelaine is a glycerol of azelaic acids, and, in the context of the invention, can also be called TriAza. In the context of the invention, the terms Triazelaine, TriAza, glycerol of azelaic acids, azelaic triglyceride, Glycerol Triazelaine, Glycerol triazelate, azelaic acid triglyceride, are interchangeable, derivatives of TriAza may comprise monoacylglycerol of azelaic acid-Mon-Aza, diacylglycerol of azelaic acid-Di-Aza, Polyazelayl glycerol-PolyAza-azelaic polymers as shown on FIGS. 4-6. As used herein, the term “plant oil source” (olive argan and apricot—is not vegetable), or “vegetable oil source” refers without limitation to plant oils or lipid extracts of plants that contain lipids, preferably triglycerides or fatty acids with carbon chain of C10-C22 with cleavable unsaturated double bond of carbon 5, 7 or 9. Most preferably Olebiome composition is formulated of plant oils enriched with Monounsaturated triolein oil, with double bond on Carbon C9. Such double bond, during oxidative or epoxide, or ozonolysis, or electrolysis cleavage results in saturated fatty caid chain with uneven carbon C5-C11. According to some embodiments, vegetable oils are natural extracts of plant derived Lipid droplets organelles from fruit bodies, or plant kernels such as, without limitation: apricot oil, almond oil, or argan (Moroccan) oil; or seed oils, such as, without limitation: sunflower oil, VHOSF, cannabis, CBD, soybean, and rice oil. Preferably the comedogenic index of selected oil composition should be less than 4, more preferably less than 3, most preferably 0-2 comedogenic index.

According to some embodiments, such molecules may be produced by synthetic route, for example by trans-esterification of azelaic acid or pelargonic acid with glycerol moiety, or by biotechnological route by microbial fermentation and engineering. According to some embodiments, the composition further comprises pelargonic acid.

According to some embodiments, the invention provides an anaplerotic composition comprising Medium Odd Chain Lipids (MOCL) or derivatives thereof, wherein said MOCL comprising esterified linear, branched or glycerol esterified fatty acids.

According to some embodiments, the composition is a topical composition.

According to some embodiments, the composition is a food grade composition. In the context of the invention, the term “food-grade” is meant to be understood as a material/substance/media that is safe for human consumption.

According to some embodiments of the above composition, the composition is a solid composition, a semi-solid composition, or a liquid composition.

According to some embodiments of the above composition, the composition may be in the form of liquid, semi-solid, semi-liquid, gel, oil, colloid, emulsion, suspension, cream-like, emulsion, soap like, foam like, solid, granulate, particulate, coating on solid layer, or other functional consistency.

According to some embodiments, the compositions may have preservative properties.

According to some embodiments, the invention provides a process for the preparation of the above compositions, comprising oxidating triolein of the olive oil without hydrolyzing the acyl glycerol bond.

According to some embodiments, the Triazelaine content in the above composition is between 1% to 100%; 5% to 95%; 10% to 80%, 15% to 75%.

According to some embodiments, the invention provides an anaplerotic composition comprising Medium Odd Chain Lipids (MOCL) or derivatives thereof, wherein said MOCL are of a non-animal source.

According to some embodiments, the invention provides anaplerotic Olebiome composition comprising
a) a blend of anaplerotic MOCL esters;
b) a Triolein-enriched plant carrier oil, and
c) at least one lipophilic antioxidant and/or antiaging ingredient.

According to some embodiments of the above anaplerotic compositions the blend of MOCL esters is at least one of:
a. enriched with glycerol and non-glycerol esters of nonanoic monocarboxylic and dicarboxylic acids, or any precursors and derivatives thereof;
b. comprises a plurality of nonanoic fatty acid derivatives and/or precursors, wherein the precursors and/or derivatives are selected from heptanoic acid, dicarboxylic nonanoic acid, and/or at least partially oxidized Triolein derivative;
c. comprises a plurality of nonanoic fatty acid ester derivatives, wherein said derivatives comprise a partially oxidized dicarboxylic derivative of Triolein, and wherein said partially oxidized derivative of Triolein is selected from azelaic acid, ester of azelaic acid, diester of azelaic acid, or any combination thereof; or, any combination thereof.

According to some embodiments, the above anaplerotic compositions are anhydrous, and the total MOCL esters concentration in the anaplerotic Olebiome composition is between 25% to 70%. In some embodiments, the total MOCL esters concentration in the anaplerotic Olebiome composition is 25%, 30%, 35%, 40%, 45%, 50%, 55%, 60%, 65%, or 70%.

According to some embodiments of the above anaplerotic compositions, the Triolein-enriched plant carrier oil is selected from the group consisting of olive oil, apricot oil, argan oil, almond oil, sunflower oil, VHOSF, cannabis oil, CBD oil, soybean oil, avocado oil, and rice oil, sesame oil, and mixture of thereof. According to some embodiments, Triolein-enriched plant carrier oil is edible plant carrier oil.

According to some embodiments of above the anaplerotic compositions, the concentration of the Triolein enriched plant carrier oil is between 25% to 85%. According to some embodiments, the concentration of the Triolein enriched plant carrier oil is 25%, 30%, 35%, 40%, 45%, 50%, 55%, 60%, 65%, 70%, 75%, 80%, or 85%.

According to some embodiments of the above anaplerotic compositions, the at least one lipophilic antioxidant ingredient is lipophilic anti-aging agent. A non-limiting list of the lipophilic anti-aging agent includes lipid soluble derivative of ascorbic acid, ascorbyl palmitate, mitochondrial/longevity-boosting ingredient, or any combination thereof. According to some embodiments, the composition comprises two lipophilic anti-aging agents. According to some embodiments, the two lipophilic anti-aging agents are, without limitation, ascorbyl palmitate and coQ10.

According to some embodiments of the above anaplerotic compositions, Triolein-enriched plant carrier oil comprises at least 30% by weight of Triolein. According to some embodiments, Triolein-enriched plant carrier oil comprises at least 30%, at least 35%, at least 40%, at least 45%, at least 50%, at least 55%, at least 60%, at least 65%, at least 70%, at least 75%, at least 80%, at least 85% by weight of Triolein.

According to some embodiments, the above anaplerotic Olebiome compositions comprise less than 30% of hydrophilic ingredients and free of pure water as a carrier. According to some embodiments the above anaplerotic Olebiome compositions comprise less than 30%, less than 28%, less than 25%, less than 23%, less than 18%, less than 15%, less than 13%, less than 10%, less than 8%, less than 8%, less than 3%, less than 1%, of hydrophilic ingredients.

According to some embodiments, the above anaplerotic Olebiome compositions further comprise at least one SPF agent, and, optionally, at least one excipient and/or formulation stabilizer complex, and wherein the excipient is a single- or a multiple-component excipient-stabilizer complex selected from “green” amphiphilic surfactant, biopolymer, or any combination thereof. As used herein, the term “SPF agent” refers without limitation to any substance having Sun Protection Factor. In the context of the invention, SPF agent can be a pre-mix, a raw material, substance in any suitable form, or any end-user cosmetic and/or therapeutic product designed for topical application, either available or not available commercially.

According to some embodiments, the above anaplerotic Olebiome compositions are in the form of oil, oil enriched emulsion, microemulsion, spray, soap, balm, foam, cream, oleogel, organogel, waxes blend, and solid stick with wax.

According to some embodiments, the invention provides a method of enhancing the sun protection factor (SPF) of an SPF agent, comprising combining the SPF agent with any of the above anaplerotic Olebiome compositions. According to some embodiments, combining the SPF agent with the anaplerotic Olebiome composition comprises applying to a sun exposed area of the skin the SPF agent and an amount of the anaplerotic Olebiome composition effective to enhance the sun protection factor (SPF) of the SPF agent. According to some embodiments, the anaplerotic Olebiome composition and SPF the agent are applied simultaneously or consequently. In the context of the invention, the above anaplerotic Olebiome compositions can be applied, without limitation, as layering as a serum/primer, coating layer before use of SPF agent, add-mixed with SPF agent, or preformulated with SPF agent.

According to some embodiments of the above method, the sun protection factor (SPF) of an SPF agent is enhanced by between 15% to 55%. According to some embodiments, the sun protection factor (SPF) of an SPF agent is enhanced by 15%, 20%, 25%, 30%, 35%, 40%, 45%, 50%, or 55%.

According to some embodiments, the invention provides a method of preventing and/or treating symptoms associated with at least one of at least one of
a. aging;
b. cellular oxidative stress;
c. external radiation induced oxidation; or,
d. any combination thereof,
comprising administering to a subject in need an amount of the above anaplerotic Olebiome compositions, effective to prevent and/or treat the symptoms. According to some embodiments, the above symptoms are selected, without limitation, from mitochondrial dysfunction, dryness, loss of water, loss of skin vitality, loss of elasticity, wrinkles, inflammation, microbiome imbalance, or any combination thereof.

According to some embodiments, the invention provides a process for the preparation of the above anaplerotic Olebiome compositions, comprising partially oxidating triolein of the plant carrier oil under anhydrous conditions, without hydrolyzing the acyl glycerol bond and preventing formation of lipid hydroperoxides and volatile by rancidity associated byproducts by adding lipophilic antioxidant.

According to some embodiments of the above anaplerotic compositions, the Medium Odd Chain Lipids have between 6 to 12 carbons. In one embodiment, the Medium Odd Chain Lipids have between 7 to 9 carbons. In one embodiment, the Medium Odd Chain Lipids have 9 carbons-C9.

According to some embodiments of the above anaplerotic compositions, Medium Odd Chain Lipids are Medium Odd Chain Fatty Acids preferably in ester form, Medium Odd Chain Triglycerides or any combination thereof.

According to some embodiment, the above anaplerotic compositions comprise azelaic acid precursors, pelargonic acid, or a combination thereof.

According to some embodiments, the above anaplerotic compositions further comprise at least one active biomolecule.

According to some embodiments, the at least one active biomolecule characterized by having anaplerotic modulation properties. In the context of the invention, the term “active biomolecule” refers to any molecular entity, including chemical entity, having biological activity. As used herein, the term “chemical entity” refers, without limitation, to a physical entity of interest in chemistry including molecular entities, parts thereof, and chemical substances. In the context of the invention the term “anaplerotic modulation properties” refers, without limitation to substrate for enzymatic generation of intermediates of TCA cycle by degradation of odd chain fatty acids. Anaplerosis function refers to the process of replenishing the Krebs (TCA) cycle intermediates and restoring metabolic homeostasis, i.e., energy balance intracellularly in cytosol. TriAza+PA composition composed of Triacylglycerol of azelaic acid and pelargonic acid are vegetable oil derived source of Medium Chain Odd-chain fatty acids (MCOCFA) that are anaplerotic source, material for OCFA catabolism TriAza and PA composition obtained by disclosed methods of the current invention are anaplerotic substrates that can be used to produce intermediates that are used to replenish the oxidative capacity of the Krebs cycle. MOCL of disclosed composition and obtained by disclosed methods will be metabolized by triglycerol lipase mediated hydrolysis of TriAza and metabolizing in the body, wherein the said lipase may be microbiome lipase or human lipase that metabolize TriAza in classic dose dependent and time dependent mechanism of hydrolysis. Derived by oxidation or by ozonolysis of vegetable oil or by synthetic route, TriAza and PA enriched composition may serve, without limitation, as food additive, food carrier, functional food, and medical food. In some embodiments, the TriAza and PA enriched composition may serve as a topical composition for cosmetic and/or pharmaceutical use.

The non-limiting list of the active biomolecules of the invention includes: MOCT with any additional anaplerotic compound that serves as non-carbohydrate source for energy homeostasis balance. In addition, composition may serve as a carrier for other cell life span protectants/anti-ageing that may add cell regenerative properties through, without limitation, epigenetic, senolytic, redox modulating, autophagy modulating functions.

According to some embodiments of the above composition, TriAza fulfills the function of a preservative to prevent bacterial, yeast or any other contamination.

According to some embodiments, the above composition further comprising at least one of a surfactant, an emulsifier, a colorant, texture/viscosity-modifying excipient or/an additive, and a taste additive, and/or at least one microbiome stabilizing agent (prebiotic, postbiotic, probiotic, quorum sensing moiety), and/or redox Active agent, and/or functional biomaterial that may regenerate cell life span in conjunction with MOCT/TriAza. In some embodiments, redox potential regulating substance is selected without limitation, of Antioxidants, Vitamins, Substances with thiol-groups, Amino Acids, Peptides, Polyamines, polyphenols, Nucleotide derivatives, Quorum Sensing Substances, Phytohormones, senolytics, autophagy modulators, proteostasis modulators, epigenetic modulators, HDAC inhibitors, and anti-ageing/longevity/life span regenerative agents.

According to some embodiments, the above compositions can be caracterized as alternative “mitochondrial energy boosters” with enhanced skin “perfecting”- and antiaging sensibility. “Mitochondrial energy boosters”, ingredients that activate, enhance, improve, mitochondrial functionality, also are categorised herein as “lohgevity boosters since mitochondrial dysfunction is included in validated “hallmarks of aging” in the state of the art (ref. Jimenez AG. A revisiting of “the hallmarks of aging” in domestic dogs: current status of the literature. Geroscience. 2024 February; 46 (1): 241-255. doi: 10.1007/s11357-023-00911-5.): are coQ10 (oxidized coQ10-ubiquinone from and reduced coQ10-ubiquinol forms, and derivatives of thereof), alpha lipoic acid (α-lipoic acid), pyrroloquinoline quinone-PQQ, L-carnitine (acetyl-l-carnitine), D-ribose, phospholipids, NAD activators, branched-chain amino acids, vitamin D, creatine, β-nicotinamide adenine dinucleotide (NAD+) derivatives, such as NADH, NMN (nicotinamide mononucleotide), and other viatmin B derivatives, keto-lipids, such as MCT-medium chain triglycerides, antioxidants (resveratrol, vitamin C, E, EGCG, glutahtion and NAC-glutatione precursors, etc.), sugars, oligosaccharides, polysacharides, and SCFA. In additon, mitochondrial metabolites that serve as co-substrates for epigenetic activities, are alos categorised as mitochondrial and longevity boosters. include ATP, α-ketoglutarate (a-KG; also known as 2-oxoglutarate, 2-OG), β-nicotinamide adenine dinucleotide (NAD+), and acetyl coenzyme A (acetyl COA) Additonal mitochondrial energy booster may be substate, co-factor, by product, end product of anaplerotic reaction, or anaplerotic reagent, anaplerotic ingredients, intermediates and derivatives of thereof, for example oxolaacetate, malate, succinate, adenylsuccinate, fumarate, propionate, citrate, formic acid, asparate, pyruvate, glutamine and derivatives of thereof like, GlutaMax, polyglutamine, ketoglutarate, succinyl-CoA, propionyl-CoA and sources for propionyl-CoA, sucha as propionic acid, and derivatives as sources are the essential amino acids: valine, methionine, isoleucine, threonine, long odd chain fatty acids and derivatives of thereof. According to some embodiments, the mitochondrial energy boosters are designed to provide formulations that not only enhance mitochondrial function but also deliver desirable anti-aging effect, contributing to overall skin health and performance.

According to some embodiments, the above compositions can be characterized by fulfilling at least one of the following functions:
a. MOCL-longevity modulation a novel feature disclosed in current invention that includes enrichment neutral lipids with MOCL composed of blend at least tow, preferably three and more MOCL, as an substate for anaplerotic reaction in mitochondrial Krebs cycle, wherein said MOCL are representing in the variety of ester forms, including glycerol and glycerol forms, to provide slow enzymes/hydrolysis-controlled release of MOCL in a free fatty acids form available as a substrate to mitochondrial, and not require in most cases the carnitine transporter that is essential for long chain lipids, those esters release MOCL in free non-esterified form by host and microbiome lipase and esterases and hydrolases, and enter into “longevity boosting effect”, mitochondrial boosting anaplerotic and ketolipid generation to activate cellular energy status.
b. Microbiome balancing that includes (i) action as a delivery system for “healthy” non-virulent microbiome; (ii) being both controlling “probiotic” for lipophilic microbiome (microbial lipase and esterase see glycerol and nonglycerol esters of MOCL as “triolein-like” prebiotic-nutritional substrate, and (iii) “postbiotic” also derived by some microbiome as yeast fungi as metabolite, and (iv) providing some microbicidal “preserving” biocontrol functions selectively to virulent and non-damaging to healthy microbiome probiotic strains).
C. SPF boosting and preventing photooxidation.
d. Mitochondrial boosting, enhancement of mitochondrial function through anaplerotic TCA substrates regeneration, and ketogenic easy energy substrate. As disclosed herein, “mitochondrial boosting” and “longevity boosting” can be interchangeable since mitochondria is a major validated “hallmark of longevity” (Brand MD. The role of mitochondria in longevity and healthspan. Longev Healthspan. 2014 May 22; 3:7. doi: 10.1186/2046-2395-3-7.)
e. Supporting homeostasis and stress response by mimicking LD.

According to some embodiments, the above anaplerotic compositions comprise mainly neutral lipids, preferably triglycerides and fatty acids derivatives essentially in the form of ester, enriched with glycerol esters of MOCL mimicking intracellular organelles, such as, without limitation, LDs.

According to some embodiments, the above anaplerotic compositions comprise neutral lipids, especially TGs, preferably enriched with anaplerotic MOCT that are glycerol lipids of MOCL, as mimetics of LD for use in longevity/antiaging applications, such as delivery systems, mitochondrial protectants, and metabolic functional foods.

According to some embodiments, the above compositions are SPF boosters. As used herein, the term “SPF booster” refers, without limitation, to a composition capable of increasing the sun protection factor of sunscreens. According to some embodiments, the above SPF booster compositions allow reducing concentration of SPF agents in the cosmetic SPF product. As used herein, the term “cosmetic-SPF product” without limitation, to cosmetic commercial products with UV protection indicated on the product label as SPF number 15-50+.

According to some embodiments, the above SPF booster compositions reduce the compounding quantity of the ultraviolet absorber which is harmful to the human body.

According to some embodiments, the above SPF booster compositions increase the absorption effect of the ultraviolet absorber by between 15% to 55%, 15% to 45%; 15% to 35%, 15% to 25%. According to some embodiments, the above SPF booster compositions increase the absorption effect of the ultraviolet absorber by 15%, 20%, 25%, 30%, 35%, 40%, 45%, 50% and 55%.

According to some embodiments, the above SPF booster compositions reduce the formulation's heaviness and improve compliance for the use of high-level SPF protectants. MOCL of Olebiome composition are composed of “light” low molecular weight MOCL enriched oils that are liquid in room temperature-RT and fast absorbed onto skin surface without “greasy feeling”, this is in contrast to standard vegetable oils and also in contrast to other widely used and known for low molecular weight oils-most medium chain lipids-based formulations that are solid at RT and leave “heavy” greasy glow or spot on skin surface. Due to its “light” texture Olebiome composition easier and compliant to apply as a primer/serum layer before using SPF cosmetic products, or/and mix with said cosmetic SPF product before application/administration. In the context of the invention, “compliance” of sunscreen compositions is to provide good protection against the sun, a measure of which is the Sun Protection Factor (SPF) value, yet have satisfactory sensory perception, such as a smooth but not greasy feel upon application, posing a significant challenge due to the fact that many active sunscreen compounds, when used in concentration to reach above SPF20-30 protective factor have an oily or greasy feel, and increasing their content tends to cause the final product to suffer from that effect.

According to some embodiments, the above SPF booster compositions are superior by combining a desired compliance and photoprotection.

According to some embodiments, based on users' testimonials, blending, layering, coating of the above SPF booster compositions with commercially available SPF 20-30 cosmetics (photoprotection sufficient for several hours of sun exposure similar to SPF 40-50) are characterized by, without limitation, an improved absorption, user-friendly application, satisfactory spreadability, less white film, and the desired balance between skin moisturizing, protection, and light texture.

According to some embodiments, the above SPF booster compositions can be considered as environmentally friendly (“green”) surfactant that offers a pre-formulation raw material or can be utilized on demand as ready-to-use foundation for blending with preparations such as, without limitation, sunscreen emulsions and water-based formulations.

According to some embodiments, topical administration of the above SPF booster compositions to sun-exposed skin areas, either mixed with, coated on, or layered before the use of SPF preparations, enhances the sun protection factor (SPF) by effectively blocking ultraviolet radiation from reaching the skin. According to some embodiments, the topical administration of the above SPF booster compositions to sun-exposed skin areas enhances the sun protection factor (SPF) by between 15% to 55%, 15% to 45%; 15% to 35%, 15% to 25%. According to some embodiments, the above SPF booster compositions increase enhances the sun protection factor (SPF) by 158, 20%, 25%, 30%, 35%, 40%, 45%, 50% and 55%.

According to some embodiments, the invention provides a process for the preparation of the above SPF booster compositions, comprising mixing sunscreens, cosmetic excipients and carriers. According to some embodiments, the invention provides a method of enhancing the sun protection factor (SPF) of an SPF agent by combining the SPF agent with the above SPF booster compositions. According to some embodiments, the step of combining the SPF agent with the above SPF booster compositions comprises applying to the sun exposed area of the skin a single dose topical formulation comprising the SPF agent with the above SPF booster compositions. According to some embodiments, the step of combining the SPF agent with the above SPF booster compositions comprises applying to the sun exposed area of the skin the SPF agent the above SPF booster compositions. According to some embodiments, the SPF agent the above SPF booster compositions are applied simultaneously. According to some embodiments, the SPF agent and the above SPF booster compositions are applied consequently. According to some embodiments, application of the above SPF booster compositions to the sun exposed area of the skin precedes the application of the SPF agent. According to some embodiments, application of the SPF agent to the sun exposed area of the skin precedes the application of the above SPF booster compositions. According to some embodiments, when applied consequently, application of the above SPF booster compositions and the SPF agent to the sun exposed area of the skin may be carried out over or after a predetermined time interval. According to some embodiments, the time interval is between 1 second to 30 minutes.

According to some embodiments, the invention provides a method for preventing and/or treating an aging-associated cellular oxidative stress in a subject in need, said method comprises administering to the subject in need an effective amount of one or more of the above anaplerotic compositions.

According to some embodiments, the invention provides a method for preventing and treating external radiation induced oxidation, e.g. photooxidation, such as sun or electromagnetic radiation inducing cellular aging said method comprises administering to the mammal an effective amount of one or more of the above anaplerotic compositions.

IN the context of the invention, “Sunscreen” when applied topically to the skin, provides protection against solar radiation, i.e. exert a photoprotective effect; the effect may comprise absorbing and/or reflecting at least part of sun's ultraviolet (UV) radiation, thus protecting the skin against sun-related damages and diseases, such as erythema, sunburn, skin cancer, etc. UV filters can include broad-spectrum UV filters that protect against both UVA and UVB radiation, or UV filters that protect against UVA or UVB radiation. Non-limiting examples of UV filters include:

Hydrophillic UV filters, such as Phenylbenzimidazole Sulfonic Acid (PBSA), Sulisobenzone-sodium salt, Benzydilene Camphor Sulfonic Acid, Camphor Benzalkonium Methosulfate, Cinoxate, Disodium Phenyl Dibenzylmidazole Tetrasulfonate, Terephthalylidene Dicamphor Sulfonic Acid, PABA, and PEG-25 PABA.

Hydrophobic organic UV filters, such as Bis-Ethylhexyloxyphenol Methoxyphenyl Triazine, Butyl Methoxydibenzoylmethane (BMBM), Oxybenzone, Sulisobenzone, Diethylhexyl Butamido Triazone (DBT), Drometrizole Trisiloxane, Ethylhexyl Methoxycinnamate (EHMC), Ethylhexyl Salicylate (EHS), Ethylhexyl Triazone (EHT), Homosalate, Isoamyl p-Methoxycinnamate, 4-Methylbenzylidene Camphor, Octocrylene (OCR), Polysilicone-15, and Diethylamino Hydroxy Benzoyl Hexyl Benzoate (DHHB).

Poorly soluble UV filters, such as Methylene Bis-Benzotriazolyl Tetramethylbutylphenol, Tris-Biphenyl Triazine, Methanone, 1, 1 ‘-(1,4-piperazinediyl) bis [1-[2-[4-(diethylamino)-2-hydroxybenzoyl]phen-yl]- and mixtures thereof

Inorganic UV filters, such as titanium oxide and zinc oxide, iron oxide, zirconium oxide and cerium oxide

Mixture of UV filters: a para-aminobenzoate derivative, a salicylate derivative, a cinnamate derivative, a benzophenone or an aminobenzophenone, an anthranillate derivative, a b, b-diphenylacrylate derivative, a benzylidenecamphor derivative, a phenylbenzimidazole derivative, a benzotriazole derivative, a triazine derivative, a bisresorcinyl triazine derivative, an imidazoline derivative, a benzylmalonate derivative, a 4,4-diarylbutadiene derivative, a benzoxazole derivative, a merocyanine derivative, malonitrile or a malonate diphenyl butadiene derivative, a chalcone derivative, or mixtures thereof.

As used herein in, the term “Cosmetically acceptable carrier” refers, without limitation, to a carrier that is compatible with skin and acceptable for application to the skin of the body, especially the skin of the face.

A non-limiting list of cosmetically acceptable carriers of the invention includes water and/or water-soluble solvents, glycerin, C1-4 alcohols, organic solvents, fatty alcohols, fatty ethers, fatty esters, polyols, glycols, vegetable oils, mineral oils, liposomes, laminar lipid materials, water, or any combinations thereof.

According to some embodiments, the invention provides a method for rapid and/or immediate improvement to the appearance of the skin and preventing and/or reverting water loss at rapid onset after administration said method comprises administering an effective amount of one or more of the above anaplerotic compositions.

The term “radiation” as used herein can refer to electromagnetic wavelengths from 2×10⁷ to 108 meters (Ultraviolet), corresponding to energy levels of approximately 5 electron volts to several kiloelectron (kEv).

According to some embodiments, the above compositions can be in the form of an oil, dye, gel, spray, paste, foam, liquid, cream, lotion, or formulated into the material, said Olebiome composition “coating” can be applied to a surface, mixed within or into a material, intra- and/or inter-woven into a material, including but not limited to a naturally sourced or synthetic edible produce. The final concentration of the above compositions can be in a range from about 1.0 mg/cm² to about 500 mg/cm².

According to some embodiments of the above compositions and methods, TriAza, AZA precursor utilized as a sufficient probiotic by lipophilic microbiome.

According to some embodiments of the above compositions and methods, the invention provides use of TriAza and its derivatives as a method of inducing TriAza in situ during process of production and/or secondary metabolism in vivo.

According to some embodiments, the above anaplerotic food grade compositions are characterized by having three main components: a lipid source, a carbohydrate source and a protein source. In some embodiments, the carbohydrate source is a mixture of trehalose and xylose, wherein the xylose: trehalose ratio is between 1:4 to 1:10. In some embodiments, the protein source is selected from collagen hydrolysate, gelatin hydrolysate, and plant protein source, or any combination thereof.

An exemplary embodiment of the above anaplerotic composition is a chocolate spread and dip food. The chocolate spread and dip is made by adding hydrophilic hydrolyzed (AA-specified) protein mixture, lipid phase (fortified with TriAza), and trehalose/xylose longevity biomimetic carb sugars. A preferable carrier is cacao obtained from cocoa beans taken from tropical cacao trees. 20% or more by weight of the content of cacao as the main raw material is classified as chocolate. Chocolate is a preferable carrier, it contains caffeine that elevates the cognitive activity, composition is further fortified by anaplerotic active enhancer (AAE)-longevity metabolites.

Selected AAE are such as anaplerotic ingredients to improve mitochondrial activity, probiotics to improve Gut-brain, gut-skin, gut-muscle axes, trehalose for relieving muscle fatigue and stress and vagal modulators, such as choline, succinic acid, to activate the gut-brain axis and improve gait pattern, and optionally, serotonin, indole, tryptophane, to improve mental stability. Chocolate comprises endogenous antioxidants, such as epicatechin, catechin, tannin, polyphenol, vitamin E, and many other minerals such as calcium, magnesium, potassium, and phosphorus. According to some embodiments, the invention provides a method of balancing the microbiome of the gut in a subject in need, comprising administering to the subject the anaplerotic compositions according to one or more of the above embodiments. In the context of the invention, the term “microbiome” refers, without limitation, to the microorganisms in a particular environment (including the body or a part of the body), specifically in the GI tract. Microbiome also refers to the combined genetic material of the microorganisms in a particular environment. Microbiome is considered as a term that describes the genome of all the microorganisms, symbiotic and pathogenic, living in and on all vertebrates.

According to some embodiments, the invention provides a method for treating and/or ameliorating and/or preventing aging related processes, manifestations, and conditions. A non-limiting list of aging related processes, manifestations, and conditions includes Fatigue, frailty, sarcopenia, muscle atrophy, movement abnormality, loss of activity, cognitive loss, and impairments of microbiome function. According to some embodiments, the invention provides a method for improving lifespan and promoting longevity. According to some embodiments of the above methods, the anti-aging effect is achieved by consumption of biomimetic anaplerotic food compositions according to one or more of the above embodiments.

According to some embodiments of the above compositions and methods, the anaplerotic compositions of the invention comprise microbiome modulators such as, without limitation, probiotics, prebiotics, and postbiotics. The non-limiting list of probiotics includes Nonvirulent such strains, as Lactobacillus, and Bifidum. The non-limiting list of prebiotics includes polyphenols, flavonoids, polyamines (spermidine), amino acids that serve also anaplerotic substrate, (e.g., leucine, tryptophan, melatonin, inulin, glucans, resistant starch, preferably oxidized resistant starch are prebiotic). The non-limiting list of postbiotic additives includes SCA, TCA intermediates, indoles (e.g., IPA), vitamins and derivatives,

According to some embodiments of the above compositions and methods, the anaplerotic compositions of the invention comprise Glycerol monolaurate (GML), C9 esters/plasticizers, glyceryl monooleate (GMO), glyceryl monostearate (GMS), and glyceryl monopalmitate (GMP) as additive, which significantly modulated glycerophospholipid metabolism.

According to some embodiments of the above compositions and methods, the anaplerotic compositions of the invention comprise addition of f resistant starch (RS) most preferably-oxidized starch that was heated and then cooled during storage before eating. Preferable oxidation may undergo during TriAza formation important probiotic as it escapes from process. RS is an digestion in stomach and small intestine and it is fermented by microbiome in large intestine into short chain fatty acids SCFA, acetate, propionate and butyrate, major postbiotic and anaplerotic substrates. Growth of microbiome, such as Bacteroides species are stimulated by RS as prebiotic.

According to some embodiments of the above compositions and methods, microbiome is capable to utilize and modulate TCA and hydroxybutyrate cycles metabolites and affected by glycolysis and glutamate metabolic pathways. Lactobacteria, e.g. Lactobacillus sp., may produce B12 derivatives, TCA metabolites, lactic acid and HBA production.

According to some embodiments of the above compositions and methods, the postbiotics are selected from SCA and vitamin B derivatives (B7, B12, B6 and B3), Nicotinamide riboside (NR), and NAD+ precursor Gut bacteria produce vitamin B3 in the colon and are capable of salvaging and metabolizing vitamin B3 and its derivatives.

According to some embodiments of the above compositions and methods, the non-limiting list of metabolic and microbiome modulators includes Bifidum longum and other human microbiome strains that produce butyrates and other protective metabolites, as well as Lactobacillus reuteri, spermidine, ascorbic acid, and vitamin E.

According to some embodiments of the above compositions and methods, MOCL derivatives that have ionic carboxylic group will facilitate membrane binding and Vander Walls interaction to form colloidal and micellular structures with surfactants, and other micro and nano-particle forming excipients, therefore will improve intracellular traffic.

According to some embodiments, the above anaplerotic compositions comprise lipid formulation modulators such as, without limitation, defoamers, emulsifiers, hard fats, and other substances, to reach desirable viscosity and/or other beneficial characteristics.

According to some embodiments, the above compositions comprise additives such as, without limitation, surfactants, poloxamers, emulsifiers, starch (oxidized and bleached by excess of hydrogen peroxide during reaction of TriAza generation), CoQ10 (turns into ubiquinone form), vitamin B12 and ascorbic acid (turns to DHA, or any other acceptable additive. In some embodiments, the above additives can be added prior and/or during oxidation to co-formulate.

According to some embodiments of the above compositions and methods, emulsifier Imwitor 375 (SASOL, GmbH) may be added at the concentration of 1-10%; Brij surfactant may be added at the concentration of 2-10% during oxidation.

According to some embodiments of the above compositions and methods, natural derived additives, such as, without limitation, citrate, lactate, linoleate, GML, IMWITOR (supplied by Sasol), lecithin (Lipoid 100-3, Lipoid GMBH,), SLS, Brij, polysorbate 60 (Croda) can be added to reach desired properties and textures.

According to some embodiments, the above compositions can be in the form of balm, dressing emulsion, oil, spread, dip, paste, sauce, or any other form that may be beneficial for the applications of the invention.

According to some embodiments of the above compositions and methods, naturally occurring emulsifiers used in emulsion formulations include, without limitation, lanolin, beeswax, phosphatides, lecithin, and acacia.

According to some embodiments of the above compositions and methods, absorption bases possess hydrophilic properties such that they can soak up water to form w/o emulsions yet retain their semisolid consistencies, such as anhydrous lanolin and hydrophilic petrolatum.

According to some embodiments of the above compositions and methods, finely divided solids have also been used as good emulsifiers especially in combination with surfactants and in viscous preparations. These include, without limitation, polar inorganic solids, such as heavy metal hydroxides, non-swelling clays such as, without limitation, bentonite, attapulgite, hectorite, kaolin, montmorillonite, colloidal aluminum silicate and colloidal magnesium aluminum silicate, pigments, and nonpolar solids such as carbon or glyceryl tristearate.

According to some embodiments of the above compositions and methods, formulation enhancers can be added including without limitation, Lipids (e.g., fats, oils, waxes, fatty acids, fatty alcohols, fatty esters), humectants, naturally occurring gums and polysaccharides (e.g., acacia, agar, alginic acid, carrageenan, guar gum, karaya gum, (e.g., carboxymethylcellulose and and tragacanth), and cellulose derivatives carboxypropyl cellulose.

According to some embodiments of the above compositions and methods, synthetic preservatives include, without limitation methyl paraben, propyl paraben, quaternary ammonium salts, benzalkonium chloride, esters of p-hydroxybenzoic acid, and boric acid can be added.

According to some embodiments of the above compositions and methods, TriAza is characterized by stabilizing and/or preserving functions such as, without limitation, preventing overgrowth of Candida, Staphylococcus and Pseudomonas.

According to some embodiments, the above compositions can be enriched with additional naturally derived antimicrobial agents such as, without limitation, anti-E. coli bactericidal agents, to prevent optional in use contamination and to improve stability. According to some embodiments of the above compositions, agents such as without limitation, essential oils or microbicidal actives of thereof, such as typhimurium, carvacrol, cito, cinnamaldehyde, geraniol, terpineol, and pinene, can be added.

According to some embodiments of the above compositions and methods, antioxidants can be added to prevent deterioration of the formulation. A non-limiting list of antioxidants includes free radical scavengers such as, without limitation, tocopherols, alkyl gallates, butylated hydroxyanisole, butylated hydroxytoluene; reducing agents such as ascorbic acid and sodium metabisulfite, and antioxidant synergists such as, without limitation, citric acid, tartaric acid, and lecithin; natural ROS modulators such as, without limitation, polyphenols and terpenoids. According to some embodiments, flavonoids (quercetin, kaempferol, myricetin, apigenin, luteolin, and others) and carotenoids (β-carotene, lycopene, lutein, zeaxanthin, and others), ROS and agents protecting against DNA damage can be added to the compositions.

According to some embodiments of the above compositions, naturally derived surfactants such as, without limitation, glycerol monolaurate; non-toxic surfactants and co-surfactants such as, without limitation, Brij-35, Brij 100, Polaxomer, Brij 96, polyoxyethylene oleyl ethers, polyglycerol fatty acid esters, tetraglycerol monolaurate, or decaglycerol monocaprate can be used.

According to some embodiments, the invention provides methods for selective depletion of virulent pathologic microbiome and enhancement of protective microbiome by preserving and controlling the risk of contamination by pathogenic species. Disclosed selective methods and compositions useful for treatment/control of lipase-producing pathogenic/virulent microbiome without damaging major host protective microbiome.

According to some embodiments, the disclosed compositions can serve as preservative agent to prevent contamination and maintain sterility of food-grade compositions.

According to some embodiments, the invention provides specific compositions and additives of TriAza based microbiome biocontrol delivery system of prebiotic, post biotic and probiotics.

According to some embodiments, the invention provides a method of delivering at least one chemical entity and/or biomolecule into a food product for consumption or a raw material for the preparation of said food product, comprising adding to said food product or the raw material for the preparation of said food product an effective amount of the composition according to the embodiments of the invention. In one embodiment, said food product is a product for human consumption.

According to some embodiments, the invention provides process for the preparation of the composition according to the above embodiments. The process comprises oxidating triolein of olive oil without hydrolyzing the acyl glycerol bond.

According to some embodiments, the above compositions are used as food, and/or a functional food.

According to some embodiments of the above use, the invention is presented by most preferable process of acceptance of acyl-glycerol of Azelaic acid (9:0-diacid) nonanoic acid from triolein of olive oil.

Additional sources of odd FAs:

Additional example may serve triglyceride of unsaturated bond positioned at odd carbon number less than 12 in the chain, such as, without limitation, Aleprestic 5-Cp-5:0 5 (cyclo-pent-2-en-1-yl)-penta-noic acid, Alepric 9-Cp-9:0 9 (cyclo-pent-2-en-1-yl)-nona-noic acid, and Aleprylic 7-Cp-7:0 7 (cyclo-pent-2-en-1-yl)-hepta-noic acid.

### Additional Raw Materials to Derive MOCL

Similar utility can be functionalized by deriving triglycerides of additional unsaturated glycerides that have unsaturated bond at odd number and may be accepted from different vegetable oils and other sources.

For example, algae oils and jojoba oils may be great sources to obtain odd chain medium chain triglycerides that can be used in food.

Surprisingly, jojoba oil is used for cosmetic, personal care, and not approved for food use in the natural form, however, once double bonds will be oxidized to form carboxylic odd chain safe derivatives-extension of use towards food, veterinary and agricultural (e.g. plant bio protection) purposes. The same process can be used to obtain dicarboxylic triglycerides of adipic and sebacic acids that have been shown to have metabolic advantages in diabetic like disorders, surprisingly we have shown that Tri-basic acid can be obtained by green process of oxidation of castor oil. Moreover, polyglycerol lipids of cutin and waxes from plant can serve source of TriAza and other optional odd chain glycerides of fatty acids and may serve great anaplerotic substrate in multifunctional food compositions disclosed herein. For example, complex dimer triglycerols of azelaic acid are found in Arabidopsis’ cutin.

According to some embodiments of the above methods and compositions, the non-limiting list of C9 FAS includes: Pelargonic Acid-PA, also called nonanoic acid FA-Fatty Acid, a esters of PA and branched PA-isononanoic acid including list of potential ingredients, such as Pentaerythrityl Tetrapelargonate; Diethylene Glycol Diethylhexanoate/Diisononanoate, Ethyl Pelargonate; Diethylene Glycol Diethylhexanoate/Diisononanoate; Butylene Glycol Diisononanoate, Cetearyl Isononanoate, Cetearyl Nonanoate, Cetyl Isononanoate, Diethylene Glycol Diethylhexanoate/Diisononanoate, Dipentaerythrityl Pentaisononanoate, Ethylhexyl Isononanoate, Isodecyl Isononanoate, Isononyl Isononanoate, Isostearyl Isononanoate, Isotridecyl Isononanoate, Neopentyl Glycol Diisononanoate, Tridecyl Isononanoate, Ethylhexyl Pelargonate, Ethyl Pelargonate, Isobutyl Pelargonate, Methyl Pelargonate; Butylene Glycol Diisononanoate, Glycol Pentaerythrityl Tetraisononanoate, Propylene Glycol Diisononanoate, Pentaerythrityl Tetrapelargonate; Pelargonic Acid, Polyglyceryl-20 Octaisononanoate; Butylene Glycol Diisononanoate, Dipentaerythrityl Pentaisononanoate, Pentaerythrityl Tetrisononanoate, Propylene Glycol Diisononanoate, Pentaerythrityl Tetrapelargonate, (E, Z)-2,6-nonadienal; trans-2, cis-6-nonadienal; Heptanoic acid, ester with 2,2-dimethyl-1,3-propanediol; Ethyl pelargonate; nonanal-C9 aldehyde. In addition, glycerol esters of C9—are mono-di- and triglycerides of nanonoic acid-Tripelargonin, Cetearyl Isononanoate, Glycerin, Isononyl Isononanoate.

According to some embodiments of the above methods and compositions, the non-limiting list of TGs includes: heptanoic acid and esters of thereof including Triheptanoin TG7, Dicarboxylic C9 azelaic acid and salts and di- and monoesters; glycerol esters, Mono-Aza, Di-Aza, Poly-Aza, and Triazelaine, Tripelargonin, Triheptanoin, other analogues of C9 glycerol non-glycerol esters and dicarboxylic azelaic acid, such as diethyl azelate (De-Aza).

According to some embodiments of the above methods and compositions, oil enriched with Triolein is used as sebum antiaging replacement therapy. In some embodiments, the oil is characterized by comedogenic index of 0-2 max (out of 5 grades of comedogenicity). The non-limiting list of oils includes argan oil, apricot kernel oil, rose hip oil, Simmondsia Chinensis (Jojoba) Seed Oil, rose flower oil (Rosa Damascena Flower Oil), Olea Europaea (Olive) Fruit Oil, most preferably olive oil BP certified, castor oil, coconut oil, soybean oil, canola oil, rapeseed oil, corn oil, cottonseed oil, peanut oil, safflower oil, sesame oil, sunflower oil (Helianthus Annuus (Sunflower) Seed Oil), linseed oil, palm kernel oil, tung oil, jatropha oil, camelina oil, pennycress oil, wheatgerm oil, pistachio oil, poppy oil, pine oil, Persea Gratissima (Avocado) Oil, hazel nut oil, grapeseed oil, colza oil, cade oil, peach kernel oil, coffee bean oil, Michelia Alba Leaf Oil, Silybum Marianum Seed Oil, Camellia Japonica Seed Oil, Carnosine, and palm oil. In some embodiments, the carrier oil is loaded with oleic acid in an amount of at least to 30% of total content of carrier oil and said oleic acid in carrier oil has functional activity in improving penetration of active ingredients into skin layer so will be bioavailable in epidermis and dermis to enter intracellular and provide desired functionalities. In some embodiments, the carrier oil comprises endogenous antioxidants preferably vit E-tocopherol, and phenols.

In additional embodiments oxidative treatment of Triolein or Triolein enriched oil without hydrolyisis byozonolysisand/or heating, and/or epoxidation and oxidative cleavage of double bonds of other unsaturated fatty acids including even triglycerides and/or diglycerides of polyunsaturated fatty acids that carry double bonds that can be cleaved by oxidation reaction and result in odd or even saturated derivatives with shorter chain length than precursor are important optional components that functionally can be substrate in anaplerotic reaction to regenerate metabolic intermediates in Krebs cycle to generate bioenergy.

According to some embodiments, the invention provides a product for consumption comprising and/or prepared from the above compositions. In the context of the invention, the terms “product for consumption”, “product for personal care use”, longevity booster “, mitochondrial booster”, “microbiome balance booster product” are interchangeable with the term “antiaging products” “topical product”, “dermatological product”, “anti-infective product”, “cosmetic product”, “lubricating product”, “emollient product”, “food product”, “food additive”, and a “food supplement”.

According to some embodiments, the invention provides a “Rainbow-Spread and Dip”-variety of protective food additives package design based on TriAza with anti-ageing functional actives “. TriAza, obtained by process according to the embodiments of the invention, is a natural anaplerotic substance with multifunctional properties, including stabilizing microbiome, based on the selective anti-microbial activity, regeneration of intracellular energy balance based on odd chain fatty acid-mediated anaplerotic functionality, and thus non-insulin boosting), also is used as amphiphilic carrier to food functional agents that are beneficial for general healthy food, for anti-ageing and special health indications. According to some embodiments, to simplify the utility of TriAza based foods we designed product called “Rainbow food system” which is a packed collection of specialty foods defined by color for seven days a week (each of the seven colors of the rainbow), flavor/seasoning (sweet, sweet-acidic, umami, sour, salty, savory, bitter, etc.), smell/fragrance/infusion (rose, orange, limonene, menta, vanilla, rosemary, basil etc.) and by cuisine (culture, geography area, country, etc., e.g. French, Chinese, Japanese, Greek, Italian, Spanish, Indian, etc.). An individual unit of Rainbow may be, without limitation, a box of seven tubes (tube, as a spread or seasoning sealed flasks, caps, boxes, bottles, jars, etc.) of food (each food type having a different color, according to the colors of the rainbow), each containing ˜20-50 ml, 10-25 ml of composition is TriAza −PA infused/oil, which is preferably consumed one a day over a week period. Each tube is designed to be consumed with cut vegetables, salad, or fruit, grain crackers, rice crackers, bread, tortilla, etc.

According to some embodiments, the content of each “Rainbow” tube comprises seven ingredients, which are selected according to their dietary or medicinal properties for affording the consumer improved metabolism that supports an extended lifespan, by, without limitation, activating inherent cellular and organ mechanisms for removing and waste toxins, increasing rejuvenation, while preventing accumulation of oxidative damage and stress. The seven categories of functional ingredient in a tube of TriAza based Rainbow food may be: Antioxidants/Redox modulator that preferably affect food color (natural source of: red, orange, yellow, green, blue, indigo, violet) preferably originating from Phyto protectants, polyphenols, flavins, terpenes, flavonoids, vitamin E, and chelators; senolytic activator (clearing of aged (senescent) cells from the body), partially by anti-inflammatory action (turmeric, rosemary, ginger, oregano, resveratrol, curcumin, cannabinoids, ginseng, saponins, terpenoids, flavonoids, polyphenols, Ginkgo biloba, capsaicin, genistein, kaempferol-PLA2, COX 2, AA, prostaglandin release inhibition; epigenetic modulating agents/HDAC inhibitors (readdressing balance in gene expression, genome-wide, to rejuvenate cell activity, MCT-ketone generating oils, BHB); cell reprogramming agents (Vitamin C, retinoic acid derivatives, a-Ketoglutarate precursors and derivatives); Sirtuin modulators (NMN, Resveratrol, Quercetin/rutin); Autophagy modulator (Genistein, lithium, caffeine, omega-3 polyunsaturated fatty acids, resveratrol, spermidine, vitamin D, trehalose, polyphenol (-)-epigallocatechin-3-gallate and combinations thereof); Proteostasis (protein homeostasis stabilizers) modulators (e.g. allicin, polyamines (spermidine, spermine), selenium, iodine, microelements: zinc, magnesium, anti-aggregation and anti-amyloid actives, such as gallic acid and derivatives (EGCG), disaccharides, xylitol, mannitol, polysaccharides, thiol compounds, lipoic acid, N-acetylcysteine, inhibitors-chelators, EDTA, succinates, citrates, protease fructose, etc.).

According to embodiments, the invention provides an some anaplerotic composition comprising one or more odd medium chain fatty acid (MOCL) or a derivative thereof, and optionally at least one functional enhancer. In non-limiting embodiments, we disclose here novel anaplerotic compositions, named OleBiome™® MOCT, based on co-formulation of at least two MOCL and derivatives thereof, and MOCT and precursors thereof, wherein said formulation additionally comprise at least one “functional enhancer”. A composition comprising C9 FA TriAza derivative as odd chain anaplerotic substrate in conjunction with PA obtained during the process and natural surfactant as optional “functional enhancer”, has fortified the desired functionality of OleBiome composition. OleBiome anaplerotic composition disclosed herein composed of PA and Aza derivatives with multiple bioprotective functionalities that are enhanced, adjunctive and even putatively synergistic with assistance of “functional enhancer”. The multifunctional OleBiome composition comprises at least two medium chain fatty acids or derivatives thereof wherein two MCFA of odd chain FA derivatives of nonanoic acid C7-C9 (C11-undecanoic FA, is not naturally derived can be used where natural sourcing less relevant such as in biofuel and other industrial applications), most preferably PA and Aza or the derivatives thereof.

In some preferable embodiments, the composition comprises at least two MOCL-odd medium chain fatty acids (MOCL)-derivatives and precursors thereof, and at least one functional enhancer. In non-limiting embodiments, In some embodiments both nonanoic derivatives are in fatty acid-FA form, in some compositions both nonanoic derivatives are in ester form, for example in glycerol ester form, such as monoglycerol esters, diglycerol esters or triglyceride (TG) form, in some embodiments nonanoic acids are mixed in FA and TG. Both nonanoic acids and derivatives thereof are co-formulated in a single composition using industrially available nonanoic derivatives at approved safe concentration. Most preferably, MOCL are MOCL-nonglycerol esters and MOCT-glycerol esters are derivatives of pelargonic or azelaic (nonanoic) C9 carboxylic acids and C12 (dodecanoic) MCFA is derived of glycerol monolaurate-GML. In some preferable embodiments, OleBiome composition optional glycerol esterified or derived components are TriAza derivative-Tripelargonine, Aza and PA. In optional embodiments, OleBiome composition anaplerotic MOCL ingredients can be in triglyceride or mono-di-glycerol esterification MOCL form, or non-glycerol ester derivative form, or conjugated with other derivation moiety.

According to some embodiments, the one or more MOCL are selected from the group consisting of pentanoic (—C5), heptanoic (—C7), nonanoic (—C9), undecanoic (—C11), tridecanoic (—C13), pentadecanoic (—C15), and heptadecanoic (—C17) carboxyl acids. More preferably, the MOCL are nonanoic (—C9) carboxyl acids.

According to some embodiments, the MOCLMOCL is of a natural source.

In preferable embodiments, MOCLMOCL is azelaic acid (AzA) or pelargonic acid (PA). In some embodiments, the concentration of each of Aza and PA is from about 5% to 20%. In some non-limiting examples, the co-formulation of TriAza with PA at concentrations around 5-20% each in conjunction with “functional formulation enhancers”, such surfactants, emulsifiers, self-assembly inducers, essential oils, antioxidants and stabilizers results in the enhancement of known characteristics of TriAza, and derivatives thereof, especially glycerol esters are enhanced by addition of pelargonic acid. PA and AA are natural products and metabolites of some plants, microbiome and derivatives can form multiple chemically-conjugated, branched, esterification modified etc. chemical forms.

According to some embodiments, the MOCL derivative is odd chain triglycerides (MOCT), i.e. glycerol esters of MOCL. According to preferable embodiments, the MOCT is Triazelaine. According to some embodiments, the functional enhancer is characterized by having anaplerotic fortifying, bioprotective and/or multifunctional properties. In some non-limiting embodiments, TriAza dicarboxylic FA precursor widely used drug and cosmetic agent, TriAza in conjunction with PA and functional enhancer characterized by multifunctionality, bioprotective activities and serve novel medium chain triglycerides-MCT precursor composition, named herein as Olebiome-TriAza.

According to some embodiments, the functional enhancer is selected from the group consisting of surfactants, emulsifiers, self-assembly inducers, essential oils, antioxidants, and stabilizers.

In more preferable embodiments, the functional enhancer is selected from the group consisting of triglycerides of even MCFA, classic medium chain triglycerides (MCT), e.g. glycerol monolaurate (GML), e.g. pure glycerol, or e.g. butyric acid-tributyrate (SCFA, epigenetic modulator), glyceride glucoside (moisture protectant), glycerol ester of wood rosin and/or glycerol gum (stabilizer/formulation enhancer), olive oil, cedar wood, oregano oil, rosemary oil, lavender, basil oil, rose oil, jasmine oil, citrus oil, lemongrass oil, vanilla, jojoba, pine oil, anise oil, argan oil, probiotic, prebiotic, Phyto protectants, e.g. polyphenolics), bioprotective lipophilic amphiphilic or hydrophilic entity, or any combination thereof.

In the context of the invention, the term “functional enhancer” is meant to be understood as a biomolecule having anaplerotic fortifying, bioprotective and/or multifunctional properties, such as surfactants, emulsifiers, self-assembly inducers, essential oils, antioxidants, and stabilizers. According to the present invention, a non-limiting list of functional enhancers includes MCFA derivative is a derivative of standard even MCFA C8-10, carboxylic MCFAs, more preferably: triglycerides of MCFA, classic MCT, GML, pure glycerol or tributyrate) SCFA, epigenetic modulator), glyceride glucoside (moisture protectant), glycerol ester of wood rosin or/and glycerol gum (stabilizer/formulation enhancer), or selected functionality enhancer towards specific bioprotective action, such as cosmetic, formulation structure enhancer (e.g. stabilizer-surfactant self-assembly inducer), whitening agent GML, Kojic acid), antioxidant, antiaging moiety, metabolic bio-protection, additional anaplerotic substrate, essential oils (cedar wood, oregano oil, rosemary oil, lavender, basil oil, rose oil, jasmine oil, citrus oil, lemongrass oil, vanilla, jojoba, pine oil, anise oil, argan oil), probiotic, prebiotic, phyto-protectants, e.g. polyphenolics), bioprotective lipophilic amphiphilic or hydrophilic entity. In some preferable embodiments Olebiome composition consists of two MOCL carboxylated fatty acid derivatives-PA, Aza and MCFA-glycerol monolaurate (C10 FA)-GML-lipid derived from human breast milk, etc. monolaurate and CML are known emulsifiers, anti-infective, whitening emollients and immune-protectants. Multifunctionality is of essential characteristic of “functional enhancer” that can be stabilizer, antiaging booster, antioxidant, mitochondrial booster, longevity booster, SPF booster. Surprisingly, mixtures of PA and Aza and/or derivatives thereof with GML, resulted in favorable multifunctional applications and structures.

According to some embodiments, the composition according to the embodiments further comprises at least one of a surfactant, an emulsifier, a colorant, or a taste additive.

According to some embodiments, the composition according to the embodiments is a food grade composition.

According to some embodiments, the composition according to the embodiments is a solid composition, a semi-solid composition, or a liquid composition.

According to some embodiments, the composition according to the embodiments has preservative properties.

According to some embodiments, the concentration of each Aza and PA derivatives and precursors and individual MOCL is from about 0.5%-25%, more preferably is from 5% to 20%.

According to some embodiments, the composition according to the embodiments serves as medium chain triglycerides (MCT) precursor composition. According to more preferable embodiments, the MCT is Triazelaine, or partially oxidized triolein enriched oil by “green” QC-controlled oxidation method such as ozonation, and/or, heating, and/or, bleaching oxidation, peroxidation and epoxidation and cleavage of triolein with formation of MOCL-TriAza and derivatives of thereof-such as Di-Aza, Mono-AZA, Poly-Aza, wherein a byproduct is nonanoic monocarboxylic MOCL, e.g. Pelargonic acid. MOCL, including TriAza and its derivatives and precursors can be obtained by other “green” chemical synthesis methods, e.g. esterification or/and catalytic synthesis, and biological methods—e.g. microbial—lipase biosynthesis.

According to some embodiments, the invention provides a method for improving aging and oxidative stress associated symptoms, such as skin disorders, CNS disorders, inflammation, dryness, itch, irritation, wounds, microbiome disbalance, mucositis, metabolic disorders, e.g. fat-related condition in a subject in need, comprising administering to the subject the composition according to the embodiments of the invention.

According to some embodiments, the age-related metabolic fat-related condition is selected from the group consisting of obesity, diabetes, aging, preterm newborns nutrition, age-related sarcopenia, anorexia, chemotherapy induced-oxidative stress or/and radiation or cachexia, wherein cachexia is a side effect of cancer therapy, age-related degenerative diseases comprising weight gain and/or increase in the body mass/weight ratio, fat deposition, brown fat tissue effects, and microbiome-related disorders.

According to some embodiments, the invention provides the use of the composition according to the embodiments of the invention, as a medicament in the treatment of a metabolic fat-related condition.

According to some embodiments, the metabolic fat-related condition is selected from the group consisting of obesity, diabetes, aging, preterm newborns nutrition, age-related sarcopenia, anorexia, cachexia, wherein cachexia is a side effect of cancer therapy, age-related degenerative diseases comprising weight gain and/or increase in the body mass/weight ratio, fat deposition, brown fat tissue effects, and microbiome-related disorders.

According to some embodiments, the invention provides topical anaplerotic compositions.

According to some embodiments, the above and foregoing topical anaplerotic compositions are suitable for the treatment of a variety of skin abnormalities associated, without limitation, with inflammation, microbiome disbalance and/or aging.

According to some embodiments, the above and foregoing topical anaplerotic compositions are based on MOCL and derivatives thereof. In one embodiment, the above compositions are based on C9 MOCL.

According to some embodiments, the above and foregoing topical anaplerotic compositions are based on the combination of C9 MOCT/MOCL and derivatives thereof.

According to some embodiments, the above and foregoing compositions comprise Triolein in carrier oil and (ii) as an active anaplerotic ingredient-MOCT-the TG and fatty acid moieties, each with 9 carbon atoms (C9 MOCL). In some embodiments the acids are nonanoic carboxylic acid and/or dicarboxylic nonanoic fatty acids or derivatives of thereof.

According to some embodiments, the above and foregoing compositions formulated as MOCL mixture of aliphatic esters such as, without limitation, fatty acid ethyl esters, triglycerides (TG) triacylglycerols (TAG), diacyl glycerols (DAG), monoacyl glycerols (MAG), and derivatives thereof.

According to some embodiments, the above and foregoing compositions are stable for at least 6 months, preferably 10 months, most preferably 12 months or greater than 12 months. In some embodiments, the compositions are stable for 24 months. In some embodiments, the composition comprises high concentration of carrier oils rich with endogenous inhibitors of Free Radicals and antioxidants, such as, without limitation, lipophilic antioxidants, ascorbyl palmitate, vitamin E, tocopherols, carotenoids, phenols, and tocopherols.

According to some embodiments, the above and foregoing compositions further comprise at least one active agent suitable for promoting antiaging functionality or sustaining the aging process. A non-limiting list of agents includes ROS/FR inhibitors (e.g. antioxidant, chelators, vitamin E and derivatives, vitamin C and derivatives), carnitine, metabolic modulators (such as Krebs cycle/TCA substrates, additional anaplerotic substrates, glucose modulator-2 deoxyglucose-2DG, ketogenic substrates, MCT), energy maintaining agents (such as B12, coQ10, ATP modulators and derivatives, nicotinamide derivatives, nicotinamide adenine dinucleotide (NAD), nicotinamide riboside, nicotinamide mononucleotide), senolytics and senomorphics, SIRT modulators (e.g. resveratrol, quercetin, pterostilbene, oleic acid), autophagy regulators (e.g. polyamines), mTOR inhibitors (e.g. caffein, fisetin, EGCG, telomere modulators), healthy microbiome balance inducers (e.g. probiotics, prebiotics, postbiotics, quorum sensing modulators), proteostasis promoters (e.g. nucleic acids stabilizers, DNA stabilizers, RNA stabilizers, spermidine), epigenetic modulators (e.g. Histone deacetylases (HDACs), short chain fatty acids, butyric acid derivatives, histone methyl transferases (HMTs), DNA methylation inhibitors, stilbens), hormone replacement agents (e.g. estrogen, DHEA, testosterone, oxytocin), anti-inflammatory agents/(AHR inhibitors, polyphenols, phytosterols, steroids, flavonoids and indoles). According to some embodiments of the above and foregoing compositions, the concentration of the at least one active agent suitable for promoting antiaging functionality or sustaining the aging process is at least 1%, preferable 2, 5%, most preferable 5% or greater. In some embodiments, at least 50% of the activity of the at least one active agent suitable for promoting antiaging functionality or sustaining the aging process is impacted/affected by the endogenous properties of selected carrier oil composition. In the context of the invention, the phrase “impacted/affected by the endogenous properties of selected carrier oil composition” is meant to be understood as level of intrinsic natural anti-ageing compounds in selected carrier oil, such as antioxidants (e.g. tocopherols, carotenes), polyphenols (e.g. coumarin), terpenes (e.g. pinenes), level of internal fatty acids with antiaging properties (odd chain fatty acids, oleic acid, myristic acid) etc.

According to some embodiments of the above and foregoing compositions, the carrier oil constitutes 50% to 90% of the composition. In some embodiments, the carrier oil constitutes 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% of the composition. In some embodiments the oil carrier TG is Triolein.

According to some embodiments of the above and foregoing compositions, C9 MOCTA/OCFA constitutes between 5% to 25% of said composition. In some embodiments, the C9 MOCTA/OCFA constitutes 5%, 6%, 7%, 8%, 9%, 10%, 11%, 12%, 13%, 14%, 15%, 16%, 17%, 18%, 19%, 20%, 21%, 22%, 23%, 24%, 25%.

According to some embodiments of the above and foregoing compositions, at least one active agent suitable for promoting antiaging functionality or sustaining the aging process, may constitute 5% to 25% of said composition. In some embodiments, at least one active agent suitable for promoting antiaging functionality constitutes 5%, 6%, 7%, 8%, 9%, 10%, 11%, 12%, 13%, 14%, 15%, 16%, 17%, 18%, 19%, 20%, 21%, 22%, 23%, 24%, 25%.

According to some embodiments, the above and foregoing anaplerotic compositions comprise: a first component selected from triglycerol, diglycerol, monoglycerol of pelargonic acid, triacylglycerol of Azelaic acid, or a mixture of thereof; and a second component selected from azelaic acid, pelargonic acid, and esters thereof.

According to some embodiments of the above and foregoing anaplerotic compositions, the mol/mol ratio of MOCT-C9 to MOCL-C9 is from 10:90 to 90:10.

According to some embodiments of the above and foregoing anaplerotic compositions, the combined amount of MOCT-C9 and MOCL-C9 make up least 5 mol % of the lipids in the composition.

According to some embodiments of the above and foregoing anaplerotic compositions, the mol/mol ratio of MOCT-C9 to MOCL-C9 is 20:80 to 85:15; 20:80 to 80:20; 30:70 to 85:15; 40:60 to 75:25; 50:50 to 70:30; 50:50 to 67:33; 55:45 to 65:35; 58:42 to 62:38.

According to some embodiments of the above and foregoing anaplerotic compositions, the total amount of triglycerides and fatty acids corresponds to: (i) C9 precursors and derivatives, such as, without limitation, pelargonic acid, azelaic acid, nonanoic esters; triglycerides such as, without limitation, Triheptanoin, Tripelargonine, and TriAza) and (ii) lipids of carrier oil. In some embodiments, the lipids of carrier oil are in the amount of at least about 30%, more preferably about 40%, most preferably about 50% or greater of total lipids of said compositions.

According to some embodiments, the invention provides topical anaplerotic compositions based on C9 MOCL and derivatives thereof for the treatment of skin abnormalities associated with aterations in sebaceous microbiome. A non-limiting list of abnormalities associated with aterations in sebaceous microbiome includes redness, irritation, acne, seborrheic disorder, eczema, atopic dermatitis inflammatory symptoms, overdryness or oilness of the skin, non-sooth/even surface or abnormal pores, uneven or hyperpigmentation, itching, loss of skin integrity, elasticity, or loss of fat, dryness, wrinkles-“aged appearance”.

According to some embodiments, the invention provides topical anaplerotic compositions based on C9 MOCL and derivatives thereof for the treatment of skin abnormalities associated mitochondrial dysfunction.

According to some embodiments, the invention provides topical anaplerotic compositions based on C9 MOCL and derivatives thereof for preventing skin abnormalities associated impaired lipogenesis and mitochondrial dysfunction.

According to some embodiments, the invention provides topical anaplerotic Triglecyride (TG) lipids-based and fatty acids (FA) precoursors enriched compositions for preventing and/or improving and/or treating and/or repairing impaired lipidome and mitichondrial dysfuntion.

According to some embodiments, the invention provides anaplerotic topical compositions comprising Triolein in carrier oil and a combination of MOCL with lipophylic antiageing compound, more preferably antioxidant, most preferably-ascorbyl palmitate.

According to some embodiments, the above and foregoing compositions provide improved penetration of C9 FAs into the adult human skin.

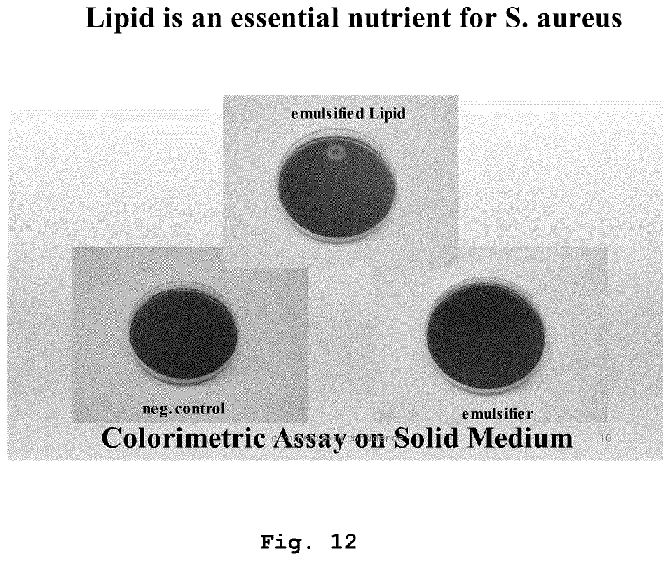

According to some embodiments, the invention provides a sebum replacement therapy based on active anaplerotuc compositions: sebum is Triolein enriched lipid lubrication of the skin is reduced with aging, loss of sebum cause microbiome disbalance (sebum comprise Triolein-we proven in FIG. 12—is essential and sufficient prebiotic for lipophilic microbiome) and dryness-Olebiome anaplerotic composition replace sebum. Also sebum comprise LD neutral lipids droplets organelles released from sebaceous glands, Olebiome anaplerotic composition is LD mimetic and therefore improve skin lubrication-protect form water loss as proven by TEWL assay, and improve microbiome balance . . .

According to some embodiments, the invention provides compositions comprising TriAza for the treatment of skin inflammation, skin disorders associated with activity of lipases secreted by microorganisms, and impaired microbiome.

According to some embodiments, the invention provides a method of treating and/or preventing and/or slowing progression of conditions associated with inflammation, microbiome disbalance and/or aging.

According to some embodiments, the above method comprises administering to a subject in need an affective amount of topical anaplerotic compositions according to one or more of the above embodiments.

According to some embodiments of the above methods and compositions, triozonoids can be used as a precursor of TriAza and PA, that in situ will be oxidized to TriAza by acidic pH, oxygen, Zn, or hydrogen peroxides, or other hydroxyl or superoxide donors to decompose ozonides to carboxylic aliphatic derivatives, such as TriAza.

According to some embodiments of the above methods and compositions, the non-limiting list of C9 FAS includes: Pelargonic Acid-PA, also called nonanoic acid FA-Fatty Acid, a esters of PA and branched PA-isononanoic acid including list of potential ingredients, such as Pentaerythrityl Tetrapelargonate; Diethylene Glycol Diethylhexanoate/Diisononanoate, Ethyl Pelargonate; Diethylene Glycol Diethylhexanoate/Diisononanoate; Butylene Glycol Diisononanoate, Cetearyl Isononanoate, Cetearyl Nonanoate, Cetyl Isononanoate, Diethylene Glycol Diethylhexanoate/Diisononanoate, Dipentaerythrityl Pentaisononanoate, Ethylhexyl Isononanoate, Isodecyl Isononanoate, Isononyl Isononanoate, Isostearyl Isononanoate, Isotridecyl Isononanoate, Neopentyl Glycol Diisononanoate, Tridecyl Isononanoate, Ethylhexyl Pelargonate, Ethyl Pelargonate, Isobutyl Pelargonate, Methyl Pelargonate; Butylene Glycol Diisononanoate, Pentaerythrityl Tetraisononanoate, Propylene Glycol Diisononanoate, Pentaerythrityl Tetrapelargonate; Pelargonic Acid, Polyglyceryl-20 Octaisononanoate; Butylene Glycol Diisononanoate, Dipentaerythrityl Pentaisononanoate, Pentaerythrityl Tetrisononanoate, Propylene Glycol Diisononanoate, Pentaerythrityl Tetrapelargonate, (E, Z)-2,6-nonadienal; trans-2, cis-6-nonadienal; Heptanoic acid, ester with 2,2-dimethyl-1,3-propanediol; Ethyl pelargonate; nonanal-C9 aldehyde. In addition, glycerol esters of C9-are mono-di- and triglycerides of nanonoic acid-Tripelargonin, Cetearyl Isononanoate, Glycerin, Isononyl Isononanoate.

According to some embodiments of the above methods and compositions, the non-limiting list of TGs includes: heptanoic and esters of thereof including Triheptanoin TG7, acid Dicarboxylic C9 azelaic acid and salts and di- and monoesters; glycerol esters, mono-di and Triazelaine; Tripelargonin, Triazelaine, Triheptanoin, analogues of C9 glycerol esters and dicarboxylic azelaic acid.

According to some embodiments of the above methods and compositions, oil enriched with Triolein is used as sebum antiaging replacement therapy. In some embodiments, the oil is characterized by comedogenic index of 0-2 max (out of 5 grades of comedogenicity). The non-limiting list of oils includes argan oil, apricot kernel oil, rose hip oil, Simmondsia Chinensis (Jojoba) Seed Oil, rose flower oil (Rosa Damascena Flower Oil), Olea Europaea (Olive) Fruit Oil, most preferably olive oil BP certified, castor oil, coconut oil, soybean oil, canola oil, rapeseed oil, corn oil, cottonseed oil, peanut oil, safflower oil, sesame oil, sunflower oil (Helianthus Annuus (Sunflower) Seed Oil), linseed oil, palm kernel oil, tung oil, jatropha oil, camelina oil, pennycress oil, wheatgerm oil, pistachio oil, poppy oil, pine oil, Persea Gratissima (Avocado) Oil, hazel nut oil, grapeseed oil, colza oil, cade oil, peach kernel oil, coffee bean oil, Michelia Alba Leaf Oil, Silybum Marianum Seed Oil, Camellia Japonica Seed Oil, Carnosine, and palm oil. In some embodiments, the carrier oil is loaded with oleic acid in an amount of at least to 30% of total content of carrier oil and said oleic acid in carrier oil has functional activity in improving penetration of active ingredients into skin layer so will be bioavailable in epidermis and dermis to enter intracellular and provide desired functionalities. In some embodiments, the carrier oil comprises endogenous antioxidants preferably vit E-tocopherol, and phenols.

As used herein, the phrase “slowing and/or preventing progression” refers, without limitation, to the influence of the treatment on the clinical course of the disease or the condition. For example, in the case of acne, illness severity ranges from mild to severe, while mild disease is categorized as <30 total lesions count to moderate disease is categorized as 30-125 of total lesions count; severe disease has manifestations of >125 of total lesions count. In the context of the invention, the proposed therapy is aimed at slowing and/or preventing the transition from mild to severe illness. The “slowing and/or preventing” progression of the condition according to the embodiments of the above method may be measured using any appropriate questionary, method, scale, diagnostic tool, or any other means that are known in the art or acceptable by the relevant functions and professionals. The term “preventing” might, but does not necessarily, means recovery from the illness. As such, the term “preventing” relates to the situation when the patient does not present symptoms and/or signs and/or manifestations of the next “stage” of illness severity as defined by the appropriate and acceptable parameters for the specific disease condition. The term “slowing”, or attenuating can, without limitation, prolong the time of transition into the next “stage” of illness severity, thus providing greater window of opportunity for extensive care and recovery.

According to some embodiments, the invention provides a method of treating an inflammatory condition of the skin in a subject, comprising administering to the subject the topical composition according to the above embodiments. In one embodiment, the inflammatory condition of the skin is associated with a lipase-producing microorganism, including, without limitation, Pseudomonas spp., Cutibacterium acnes, Staphylococcus, and Corynebacterium.

According to some embodiments, the invention provides a method of treating an inflammatory condition of the skin such as, without limitation, eczema, acne, atopic dermatitis, tissue necrosis, skin sores, psoriasis, cellulitis, fungal infection, gangrene, and disorder of the pilosebaceous unit, comprising administering to the subject the topical composition according to the above embodiments.

According to some embodiments, the invention provides a method of treating localized skin inflammation comprising administering to the site of the inflammation the topical composition according to the above embodiments. In the context of the invention, the term “localized skin inflammation” is meant to be understood as local inflammatory response, which is limited to a certain, well-defined area/portion/region of the skin affected by the harmful stimulus. Localized skin inflammation according to the embodiments of the invention may appear simultaneously in several parts of the body.

According to some embodiments, the invention provides a method of balancing the microbiome of the skin in a subject in need comprising administering to the subject the topical composition according to the above embodiments. In the context of the invention, the term “balancing the microbiome” is meant to be understood as maintaining the possibility of microbiome homeostasis to control by selectively targeting target the desired opportunistic or/and pathogenic species of microbiome which causes the production of the triglyceride lipase as a virulent factor causing disease or disorder and thus reaching desired effect in reduction of disease or disorders symptoms while keeping microbiome balanced.

According to some embodiments, the invention provides a method of treating a condition associated with disbalance of skin microbiome in a subject in need comprising administering to the subject the topical composition according to the above embodiments.

According to some embodiments, the invention provides a method of maintaining the balance of skin microbiome in a subject in need comprising administering to the subject the topical composition according to the above embodiments.

According to some embodiments, the invention provides a method of preventing aging of the skin in a subject in need comprising administering to the subject the topical composition according to the above embodiments. Classical manifestations of aging include genomic instability and telomere attrition, epigenetic alterations, and loss of proteostasis, deregulated nutrient-sensing, mitochondrial damage and dysfunction, cellular senescence, stem cell exhaustion/dysregulation, and altered intercellular communication. In the context of the invention, the term “aging of the skin” is interchangeable with such terms as “skin abnormalities/diseases/disorders associated with aging”, and refers, without limitation, to phenomena such as decline of both absolute and relative amounts of sebaceous TG, skin dryness, lack of brightness, loss of skin microbiome, wrinkling, loss of elasticity, laxity, rough-textured appearance of the skin, sebaceous glands (SG) senescence and SG atrophy, and disturbance of skin barrier. The aging process is usually accompanied by mitochondrial dysfunction, phenotypic changes in cutaneous cells as well as structural and functional changes in extracellular matrix components such as collagens and elastin.

The present invention further provides a lipase-hydrolyzable (lipase-mediated) site-targeting sytem releasing upon hydrolysis an inert, or non-inert, carrier compound (referred to as “carrier component”) and an active compound (referred to as “active ingredient”), selected from the group consisting of:

According to some embodiments, a lipase-hydrolyzable triglyceride ester comprises an inert carrier component (glycerol moiety) and an active component (fatty acid moiety). For example, a component, such as azelaic and/or retinoic acid is chemically attached to the glycerol backbone to form tri-retinoic, tri-azelaic and mixed di-retinoic, mono-azelaic, or di-azelaic, mono-retinoic ester derivatives.

In preferable embodiments, the multifunctional Olebiome formulations of glycerol (stabilizer and skin barrier enhancer), glycerol esters (e.g. GML-glycerol monolaurate-natural emulsifier and bioprotectant), glycerides (diacylglycerides, metabolic modulators, glycerides of glucose-moisture and osmoprotectant) and triglycerides (MCT-emollients, ketogenic agents, stabilizers) with MOCL and/or MOCT are compliant for scale up and practical applicability. The resulted Multiple Functions achieved by the composition disclosed herein are selected from:

1. Structural effect on formulation resulted in oil-based micro emulsion (>5 microns) when prepared by industrial close loop vacuum reactor process (e.g. EKATO vesicle), sub microemulsion (<1-5 mcn) produced by homogenic sonication in bath equipment (Sonomechanic machines), nanocolloids (200 nm-1 mcn) when processed by ultra-sonication (Ultrasonix) equipment glycerol esters impact stability, and/or alternatively mixed under high pressure homogenizing to yield uniform suspension of solid lipophilic homogeneous oil based lipophilic formulation, with addition of surfactant- or amphiphilic self-assembly formulation. The latter is based on surfactant or emulsifying abilities of selected MCFA and MCT and glycerol esters in composition.

2. Metabolic health status optimizing/modulation function is characterized by anaplerotic abilities of MOCL and ketogenic abilities of eMCFA. Metabolic functions can be measured and characterized by mitochondrial activity; AMPK activation path, ATP/AMP ratio and NADPH assay related energy assays; lipogenesis and hepatic and pancreatic enzymes activity through lipolytic modulation of MCFA; glucose in blood, gluconeogenesis; amino acids metabolism and consumption as major accepted in state of the art today anaplerotic substrates; reduced glutamate toxicity in vitro and in vivo, e.g. dependent of glutamate/glutamine enzymatic path;

3. Effect on obesity, fat liver, diabetes is measured by weight gain, fat deposition, fatty acid oxidation rate, and weight gain and ketosis level in plasma is regulated by measured: hormonal activity-suppress insulin resistance, insulin sensitivity; anabolic and catabolic parameters; adipocytes phenotype changes, etc.

4. Antiaging functionality of disclosed composition is expected is measured by effect on HDAC, and butyrate mediated epigenetic activity.

5. “Calorie restriction-mimetic”, even more appropriate functional characterization of anaplerotic Olebiome composition is a calorie substitution-a novel function is disclosed based on anaplerosis alternative energy capabilities provided by disclosed composition and is measured by effect on physiological parameters and performance during intermittent fasting, sport activities, muscle strengths and gain (body mass/weight ratio). Less calories needed to mitochondria due to regeneration of Krebs cycle substrates during anaplerosis. In such composition preferable “functional enhancers are MCT-as ketogenics, amino acid anaplerotic substrates, metabolic modulators such as metformin, SCFAs, low calorie proteins, peptides and alternative sugar substitutions sources, such as stevia, monk sugar, xylitol, trehalose, etc.

6. Bioprotective role of disclosed composition in certain medical conditions, such as preterm newborns nutrition, age related sarcopenia, anorexia and cachexia as side effects of cancer therapy, age degenerative diseases-measures weight gain, muscle and weight gain (body mass/weight ratio), fat deposition, brown fat tissue effects, etc.

7. Delivery system for lipophilic bioprotective, and healthy-ageing agents, for example Phyto protectants, such as natural bioprotectants, such as polyphenols (e.g. resveratrol), flavonoids (quercetin/rutinoside), terpenes, cannabinoids, ferulic acid, allicin, NAC, spermidine, catechins, essential oils, etc.

8. Microbiome balancing ability's function determines the optional use of selected MOCL composition as preservatives in food and cosmetic formulations based on selectivity of anti-pathogenic, and microbicidal read outs:

a) targeting microbial virulent lipase (e.g. TriAza shown anti-Corynebacterium, anti-Malassezia, anti-Candida anti-Staphylococcus and anti-Pseudomonas selective activities are enhanced when PA is present the composition.

b) Anti-Salmonella activity of pelargonic acid add to optional use of disclosed composition in
c) Immunomodulating and antiviral activities of GML
d) Microbiome balancing optional function of MCFA is used as delivery vehicle of probiotic microbiome, its postbiotics (SCFA, indoles), prebiotics, quorum sensing moieties, etc.

e) Preferably—as a delivery system for live microbiome for nutritional cosmetic and medical functional applications without risk of contamination by pathogenic microorganism)

In additional embodiments we disclose, multiple beauty related healthy skin related cosmetic functions of Olebiome composition results in following multiple functions (further enhanced by selected “functional enhancer” as exemplified below):
a) Improved whitening (kojic acid, GML)
b) improved emollient (GML, hyaluronic acid, glycerin, and derivatives)
c) improved skin barrier (elastin, osmoprotectants, wax, shea butter, ceramides, cholesterol, glycerol, GML)
d) moisturizing protection (hyaluronic acid, MCT, GML) e) microbiome skin balance (pre-, post-, probiotics, essential oils, GML)
f) anti-acne, selected functional enhancer salicylic acid, benzoyl peroxide, GML)
g) anti-rosacea (ivermectin, GML)
h) anti-inflammation (GML, lipoic acid, green tea extract, chamomile extract)
i) anti-alopecia and/or improved hair condition and growth, for example in conjunction with functional enhancers—e.g. lipase inhibitors such as Jack inhibitors, GML, minoxidil, etc.

j) anti-seborrheic functional enhancer exemplified as: corticosteroids, anti-fungal, microbiome-protective probiotic,)
k) anti-allergic (antihistamines, corticosteroids)
l) anti-melasma (AHA-alfa-hydroxy acids, hydroquinone)
m) anti-dermatitis (lanolin, antibiotics, steroids, Zn)
n) soothing enhancement (ferulic acid)
o) antiaging (collagens, retinoids, spermidine/spermine, niacin, biotin, vit C, antioxidants, exosomes cell derived agents, such as mRNA, e.g. mRNA-TERT, etc.)
p) energy burst (enhanced by coQ10, niacin, biotin, etc,)
q) odorants delivery and enhancement, e. g. pelargonic aldehyde-nonanal improve odorant properties and functionality of citral, other rose oil, natural essential oils scents improvement, pheromone enhancement (and may pelargonic azelaic acid esters especially glycerol esters may serve carrier controlled by skin microbiome lipase delivery system for odorants when formulated in Olebiome anaplerotic composition.

According to some embodiments, the invention provides a method of preventing aging of the skin in a subject in need comprising administering to the subject the topical composition according to the above embodiments. Classical manifestations of aging include genomic instability and telomere attrition, epigenetic alterations and loss of proteostasis, deregulated nutrient-sensing, mitochondrial damage and dysfunction, cellular senescence, stem cell exhaustion/dysregulation, and altered intercellular communication. In the context of the invention, the term “aging of the skin” is interchangeable with such terms as “skin abnormalities/diseases/disorders associated with aging”, and refers, without limitation, to phenomena such as decline of both absolute and relative amounts of sebaceous TG, skin dryness, lack of brightness, loss of skin microbiome, wrinkling, loss of elasticity, laxity, rough-textured appearance of the skin, sebaceous glands (SG) senescence and SG atrophy, and disturbance of skin barrier. The aging process is usually accompanied by mitochondrial dysfunction, phenotypic changes in cutaneous cells as well as structural and functional changes in extracellular matrix components such as collagens and elastin.

According to some embodiments, the active agent of the invention TriAza, exhibit multifunctionality which is based on its nature as a (a) lipid-based emollient (i.e. skin and mucosal barrier protection) and as a (b) microbiome balancing, by maintaining low risk of overgrowth of lipase producing microbiome strains.

According to some embodiments, the active agent of the invention TriAza is targeted to treat inflammation/irritation symptoms caused by at least one of the following pathological processes (1) skin barrier destroyed and/or (2) lipase overexpressing microbiome overgrowth.

According to some embodiments, the composition comprising TriAza, or C9 derivatives of thereof, provides a safe and effective “anti-irritation”, or “anti-inflammatory treatment.” Said “anti-irritation” treatment meaning: (i) reducing the severity of the irritaton, (ii) ameliorating a symptom of the irritation, and/or (iii) shortening the duration of the skin irritation, (iv) decreasing recovery time from a irritation.

According to some embodiments, the said anti-inflammatory treatment includes relief, or temporary relief of pain, redness, or discomfort due to mild to moderate skin inflammation.

According to some embodiments, the use of TriAza composition for the treatment for sunburn induced inflammation, or other kind of inflammatory symptoms, such as irritation, burning sense, itching, redness, by administering to the patient in need a combined therapeutically effective amount of TriAza and optionally OTC (over-the-counter) local analgesic (e.g. lidocaine), or/and NSAID (e.g. piroxicam), steroid (e.g. cortisone), or/an antihistamine.

According to some embodiments, the use of TriAza composition for the reduction of symptoms of microbial overgrowth induced irritation, for example Candida induced anti-irritation treatment of topical skin or mucosal irritation caused by Candida or other microbial pathogen, or mixed biofilm caused overgrowth or infection-induced by lipase-overexpressing unbalanced commensal microbiome, such as, without limitation, Candida, Pseudomonas, and Staphylococcus. Infection can be nosocomial infection caused by resistant strains (e.g. MRSA).

According to some embodiments, the active agent of the invention TriAza can be given as a single composition or as a combination with other anti-infective microbicidal or/and antibiotic treatments, such as, without limitations clotrimazole, mupirocin, streptomycin, iodine, permanganate.

According to some embodiments, the use of TriAza composition is for the treatment of a vaginal yeast infection (candidiasis) that causes irritation, discharge and intense itchiness of the tissues at the vaginal opening. Preferable use of TriAza combination for vaginal candidiasis is combined with live or freeze-dried probiotic, or/and postbiotic, or/and antifungal such as, without limitations clotrimazole agents.

According to some embodiments, the use of TriAza composition is for the prevention and treatment of vaginal inflammation symptoms caused by postmenopausal dryness, a skin barrier estrogen reduction associated with impairment, in combination with lactic acid or/and estrogen agents in single formulation or as a parallel regimen.

According to some embodiments, the use of TriAza composition is for prevention, reduction, shortening and elimination of symptoms of mucositis. According to some embodiments, the inflammation/irritation symptoms caused by mixed infection includes chemotherapy or radiation induced mmucositis cause sores in the mouth, throat, stomach, rectum, or vagina due to inflammation of the mucous membranes. According to some embodiments, the symptoms are accelerated by degraded mucosal barrier due to cancer unbalanced overgrowth of lipase-overexpressing microbiome. According to some embodiments, TriAza composition is used in combination with local anesthetics, such as, without limitation lidocaine, and/or vasoconstrictor, such as, without limitation pseudoephedrine, and/or antihistamine, such as, without limitation loratadine. According to some embodiments, another symptom of cancer therapy induced inflammation is skin dryness and inflammation symptoms.

According to some embodiments, preferable anti-mucositis regimen for the prevention of mucositis and skin barrier inflammation and overgrowth symptoms is administered (i) prior, (ii) during and (iii) post-cancer treatment.

According to some embodiments, the use of TriAza compositions for the prevention can be formulated in frozen, ice cream-like formulations to be taken immediately before or during chemotherapy, radiotherapy to protect mucosal and barrier:

According to some embodiments, TriAza compositions are administered to a patient before, during and after cancer therapy sessions for the best results. According to some embodiments, TriAza compositions should be administered prior to cancer treatment-preferably preventive eradication of lipase-expressing microbiome by application of TriAza combination, as a stand alone or in combination with other antimicrobials.

According to some embodiments, TriAza compositions are administered as cooled formulations during chemotherapy especially during radiotherapy sessions, preferably with local anesthetic and/or vasoconstrictor as above.

According to some embodiments, TriAza compositions are administered during post-treatment, and includes pro-; pre-; post-biotic for microbiome balance.

According to some embodiments, the multifunctionality of TriAza/and MOCL composition is based on: (1) regulation of cellular barrier and anti-inflammatory activity; (2) preserving cellular energetic status based on anaplerotic nature of odd chain fatty acids as substrate to Krebs cycle regeneration and as a medium chain fatty acid source for ketogenic energy; (3) microbiome balance through preventing overgrowth of lipase overexpressing commensal microbiome (Staph spp., Candida) and pathogenic species (Aspergillus; MRSA; Pseudomonas). Therefore, such multifunctionality targets three major skin ageing causes: (1) skin barrier loss translated in aging appearance related symptoms-skin dryness, pigmentation, wrinkles and loss of elasticity; mitochondrial dysfunction and microbiome disbalance that also impact “aged appearance” of mature skin.

According to some embodiments, the disclosed TriAza/C999

Olebiome compositions may be used to treat various dermatological disorders such as, without limitation hyperkeratosis, photo-aging, bums, donor site wounds from skin transplants, ulcers (cutaneous, decubitus, venous stasis, and diabetic), psoriasis, skin rashes, and sunburn photoreactive processes. According to some embodiments, the topical therapeutic compositions of this invention may also be used orally in the form of a mouth wash or spray to protect and accelerate the healing of injured oral tissue such as, without limitation mouth sores and bums, treatment of various dermatological disorders such as, without limitation hyperkeratosis, photo-aging, and sunburn photoreactive processes.

According to some embodiments, the mechanism of said treatment in the disclosed invention is directed to a method for preventing and reducing injury to mammalian cells, and increasing the resuscitation rate of injured mammalian cells, which comprises the steps of providing a therapeutic wound TriAza healing composition which comprises (a) carrier preferably vegetable oil enriched with Triolein (at least 20%) and comedogenic index less than 2, (b) MOCT/OCFA, wherein MOCT is TriAza or/and Tg9-tripelargonin, or/and Tg7-triheptanoin, and OCFA-nonanoic acids, salts and esters or diesters of thereof, branced and/or linear, and (c) a mixture of anti-ageing active agents, e.g. antioxidants or/and enhancers targeting protection of cellular barrier (e.g. carrier oils) or mitochondrial energy balance (coQ10, acyl-carnitine) or derivatives of thereof assiting for the resuscitation of injured mammalian cells, and contacting the therapeutic wound healing composition with the mammalian cells.

According to some embodiments, injury of mammalian cells which causes epidermal damage such as, without limitation lesions, incisions, wounds in which the skin is broken by a cutting instrument and lacerations, wounds in which the skin barrier (epidermal cells barrier) is broken by lesion induced by endogenous degenerative process (e.g. diabetic ulcers, immune-related psoriasis, or allergic induced pruritis, aging induced dryness and sores, inflammation); or by external, i.e.

mechanical tension (pressure wounds, shaving wounds, surgery, etc.), burns, or by photo UV radiation-gamma radiation-induced damage; or by xenobiotic infection.

According to some embodiments, the invention provides a product for consumption comprising and/or prepared from the composition according to the embodiments of the invention.

According to some embodiments, the composition according to the embodiments of the invention is selected from a pharmaceutical composition, a nutraceutical composition, and/or an edible composition.

Tripelargonine may serve as TriAza derivative, so Olebiome TriAza can be in form of Tripelargonine plus PA, or TriAza and optionally-functional enhancer, e.g. in the form of natural surfactant, or and natural oil (exemplified ozonated or/and oxidized processed olive or other vegetable oil composed of triolein in significant concentration) components and derivatives.

The present invention has surprisingly found the improvement of using glycerol ester of azelaic acid in conjunction with its by product (pelargonic acid), another odd chain C9 FA derivative, wherein both obtained by double bond oleic acid triglyceride. TriAza and PA in same composition without purification and separation and extraction steps. Therefore, during the process of TriAza oxidative production, we obtained PA and AzA derivatives at one step; herein we disclosed multifunctional composition with PA and Aza without the need of separation to obtain enhanced activities.

### EXAMPLES

### Example 1: Lipase-Mediated Activity

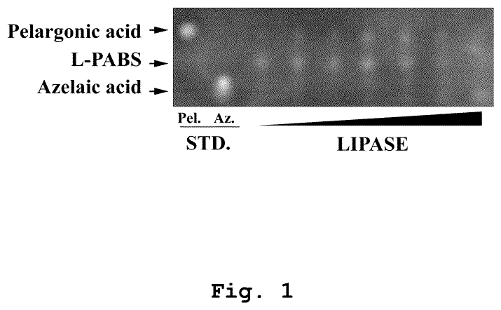

To determine the affinity of a bacterial lipase to the active ingredient according to the embodiments of the invention, the ability of the lipase to utilize TriAza as a substrate was tested. For this purpose, the commercially available lipase from P. cepacian was used. Increasing amounts of the enzyme were mixed with a fixed concentration of the substrate, and incubated for a few hours at 37° C. The enzymatic activity was monitored by TLC, by registering the production of azelaic acid. It was found that increasing concentrations of the enzyme are coupled to the gradual accumulation of azelaic acid at the expense of TriAza (FIG. 1). The results suggest that TriAza could be utilized as a substrate for bacterial lipase activity.

### Example 2: Lipase-Mediated Specificity

This example proves the controlled release of nonanoic MOCL by microbiome lipase (prove MOCL as microbiome derived metabolites-postbiotic) and host derived metabolites by intracellular and extracellular enzymatic activity of esterases that will release MOCL from glycerol nonglycerol esters, providing slow-controlled release of anaplerotic substrates of Olebiome composition ingredients.

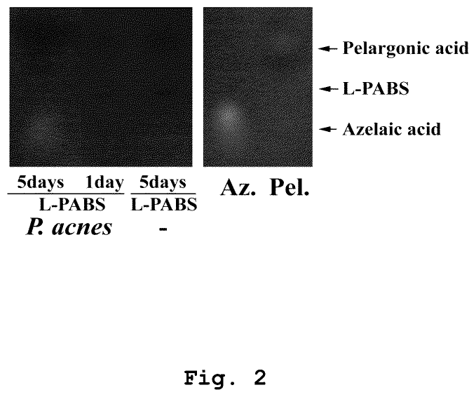

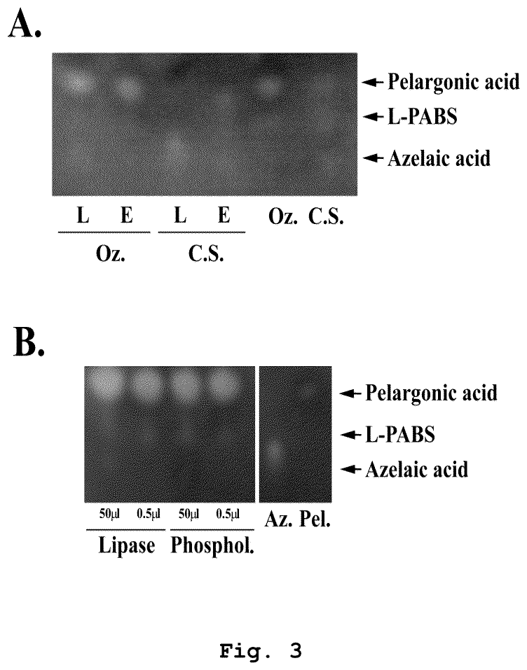

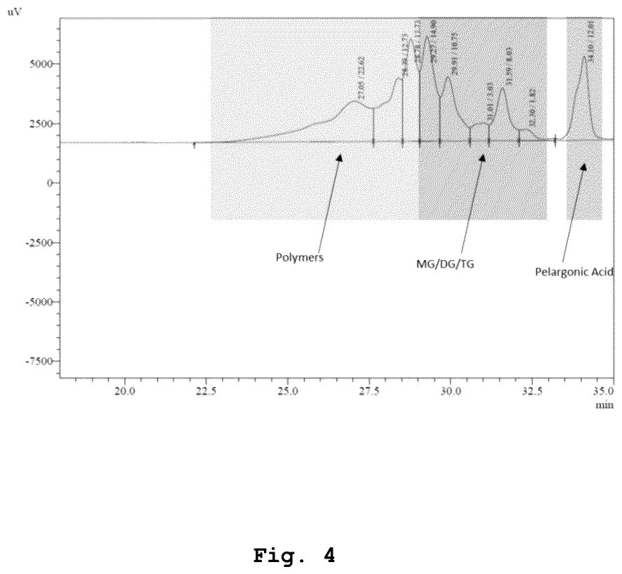

Pure pro-drug activity of designed molecules implies their high specificity towards the target enzyme. TriAza represents the glycerol ester of a specific fatty acid that might be recognized by a mammalian esterase or other triglyceride-utilizing lipase. To establish specificity of bacterial lipase to TriAza, the lipase activity from P. cepacia to that of mammalian esterase and phospholipase A2 were compared. The enzymes, in equally active amounts, were exposed to the fixed concentrations of TriAza. The extents of enzyme activities were registered according to the level of generation of azelaic acid. It was found that in contrast to the lipase, neither esterase nor phospholipase A2 could produce azelaic acid (FIG. 2 and FIGS. 3A-B). These results indicate that consumption of TriAza by bacterial lipase is highly specific.

### Example 3: Antibacterial Activity

TriAza was designed as a pro-drug of azelaic acid, a known antibacterial agent, for the treatment of skin disorders associated with pathogenic propagation of bacteria. For example, the development of Acne vulgaris is tightly associated with extensive accumulation of P. acnes in the sebaceous gland, a milieu of the disease. To determine the antibacterial activity of TriAza against P. acnes, we tested the minimal bactericidal concentration (MBC) of TriAza. The effect of TriAza was compared to the MBC of azelaic acid and benzoyl peroxide (BPO), two main competitors used in the treatment of acne. We found that TriAza, as well as the reference molecules, showed an anti-microbial activity against P. acnes ranging between 0.5-5 mg/ml (Table I). Even though all the molecules behaved similarly, the activity of TriAza was found to be 2-fold stronger than that of Azelaic acid, supporting the use of TriAza in the treatment of acne vulgaris.

TABLE 1
Activity of antibacterial agents against P. acnes

| Name | MIC* |
|---|---|
| Azelaic acid | 3.75 mg/ml |
| BPO | 0.5 mg/ml |
| TriAza | 1.95 mg/ml |

*MIC designates a minimal inhibitory concentration for tested bacteria. MIC of L-PABS is equal to its minimal bactericidal concentration (MBC). MIC of L-PABS that is demonstrated in the table characterizes an antibacterial activity of batch #5.

To compare the antimicrobial activities of various batches of TriAza we determined MIC of number of batches that were prepared with non-principal modifications. Selected batches were labeled as TriAza −3, TriAza −6, TriAza −7 and TriAza −19 and supplied to the growth medium of P. acnes. A, minimal inhibitory concentration of the batches was monitored and compared to each other. We found that all samples possess similar antibacterial activity with small deviations (Table II). For further analysis, batch TriAza −19 was chosen as a batch containing the minimal amount of pelargonic acid.

Since TriAza is designed for marketing as a soap, we decided to define an antibacterial activity of its soap. To this end, sodium salt (a soap) of the most active batch of TriAza (TriAza-19-Na) was prepared and MBC of the material was determined. We have found that the TriAza −19-Na exhibits both anti-bacterial and bactericidal activities, yet less significant than that of TriAza −19 itself (Table I). This finding indicates that soap of TriAza can be used in anti-P. acnes treatment, although with a less anti-bacterial activity. It should be stressed that despite the decreased activity of TriAza soap it is still higher than that of azelaic acid (Table 1 and 2).

TABLE 2
Activity of various TriAza batches and their soaps against P. acnes

| Name | MBC (mg/ml) |
|---|---|
| TriAza-3 | 1 |
| TriAza-3-Na | 4 |
| TriAza-6 | 2.8 |
| TriAza-6-Na | 4 |
| TriAza-7 | 2 |
| TriAza-7-Na | 6 |
| TriAza-19 | 1 |
| TriAza-19-Na | 4 |

### Example 4: Antibacterial Specificity: Range of Activity

Since TriAza possesses antibacterial activity, we decided to determine the extent of its specificity to P. acnes. For this purpose, we selected a few major skin pathogens and tested their sensitivity to TriAza. We chose Staphylococcus aureus (S. aureus) and Candida albicans (C. albicans), representing bacterial and fungal genera, respectively. Both organisms' express lipase. The bacteria and fungi were exposed to various concentrations of TriAza −19 and its sodium salt, and both the MICs and MBCs of the agents were statistically determined. We found that both organisms were sensitive to the material, with S. aureus exhibiting higher sensitivity. The level of TriAza activity was like that demonstrated against P. acnes. The slight difference in MICs of TriAza against the tested organisms can stem from the difference in their lipase activity. Our findings revealed that TriAza could be used in the treatment of various infectious diseases associated with S. aureus and C. albicans infections.

TABLE 3
Activity of TriAza and its soap against S. aureus and C. albicans *

| | MIC | MBC | MIC | MBC |
|---|---|---|---|---|
| | (S. | (S. | (C. | (C. |
| Name | aureus) | aureus) | albicans) | albicans) |
| TriAza-19 | 5 | 10 | 20 | 24 |
| TriAza-19-Na | 31 | 63 | 31 | 37 |

*** The parameters of MIC and MBC are presented in mg/ml calculations.

### Example 5: Ozonolysis and Synthesis Examples

The synthesis consists of three steps only, making it simple and optimized as ecologically safe and cost effective:

(1) OLIVE OIL→(2) OXIDATION/OZONOLYZIZ→(3) SOAP-LIKE FORMULATION (10%-20% of 3A)

This synthesis was performed by ozonolysis, an ecology-friendly method utilizing triolein, a natural material, as a substrate. Ozonolysis relies upon ozone-mediated breakage of triolein-containing double bounds that is followed by a production of COOH groups (Scheme 1). This method includes two steps: 1. exposure of triolein to the mixture of ozone and oxygen (in a solvent) and 2. oxidation of first reaction products by oxygen in acidic environment.

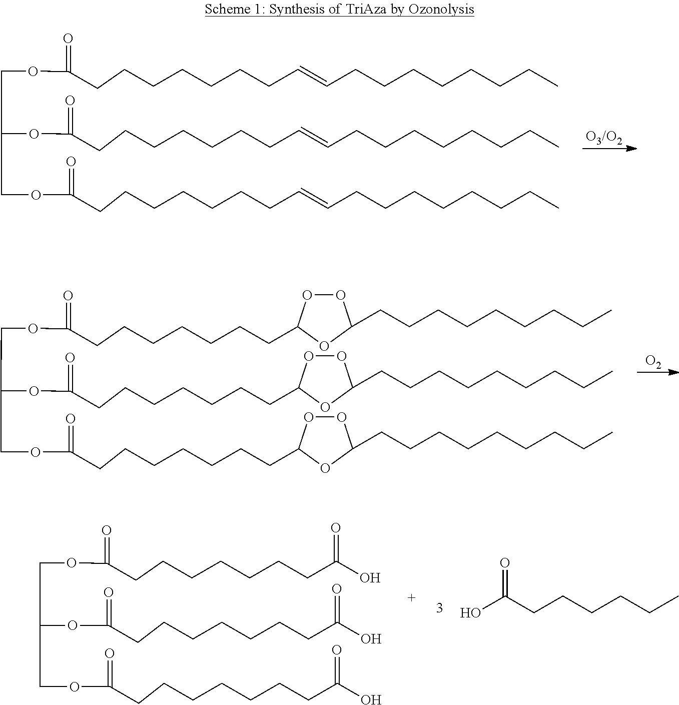

Transesterification is an additional method of current invention, is a structural lipid modification method of preparation of TriAza −to prepare TriAza −tributyrin, triolein, or other natural based selected triglyceride comprising the at unsaturated or middle chain fatty acid as a least one constituent fatty acid may be subjected to transesterification: in such reaction lipase enzymatic method may be used or a catalytic method based on chemical catalyst to be used in ester interchange may be a chemical catalyst. In ester interchange using a chemical catalyst, the constituent fatty acids will be bonded at random positions. The applicable chemical catalyst may be exemplified by alkali metal hydroxides s lithium hydroxide, sodium hydroxide and potassium hydroxide, or alkali metal alkoxides such as lithium methoxide and sodium methoxide. In the case of using a chemical catalyst, about 0.1-2 wt % of the catalyst may first be added to the mixed oil (natural triglyceride and azelaic acid, or any R selected from R description) consisting of the triglyceride, and the resulting mixture is subjected to reaction under stirring at an atmospheric or reduced pressure for 3-120 minutes at 50-270. degree. C. By performing ordinary purification steps such as washing with water, drying, decoloring, and deodorizing, the end-product can be obtained.

Substrate (TriAza) for lipase-mediated activity was synthesized. This synthesis was performed by ozonolysis, an ecology-friendly method utilizing triolein, a natural material, as a substrate. Ozonolysis relies upon ozone-mediated breakage of triolein-containing double bounds that is followed by a production of COOH groups (Scheme 1). This method includes two steps: 1. exposure of triolein to the mixture of ozone and oxygen (in a solvent) and 2. oxidation of first reaction products by oxygen in acidic environment.

The parameters that were used are solvents (dichlormethane, pelargonic acid, formic acid, water, n-propylacetate), feeding acids (pelargonic, formic, acetic, citric acids), and reaction time. In a result 17 batches were produced that were analyzed by thin-layer chromatography (TLC), nuclear-magnetic resonance (NMR), and mass spectroscopy (MS). To confirm the results produced by ozonolysis of triolein, TriAza was synthesized by either oxidation of triolein using Os04 or reacting glycerol with azelaic acid. The materials synthesized by these methods were analyzed by TLC along with the products of ozonolysis. TriAza synthesized by ozonolysis were subjected to MS, TLC, and NMR analysis. An analysis of molecular mass revealed that all synthesized batches contain mono-, di-, and triglycerides of TriAza MOCL family. In addition, triolein, TriAza −containing aldehydes, diglyceride were identified with both OH and COOH termini, triglycerides with dimmers of azelaic acid, and pelargonic acid. To further confirm the presence of ozonides in batches 3H-NMR was performed. The analysis has revealed that none of the batches contained ozonides and all batches were almost free of molecules possessing double bounds. Since triolein is the only double bond containing molecule, it was concluded that the substrate is not completely utilized in the reaction. Moreover, an absence of ozonides in the samples is evident to their destruction and producing modified forms of medium-chain triglycerides, optionally obtained MOCL, such as TriAza derivatives: e.g. Mon-Aza, Di-Aza, Poly-Aza represented in Scheme 2 below:

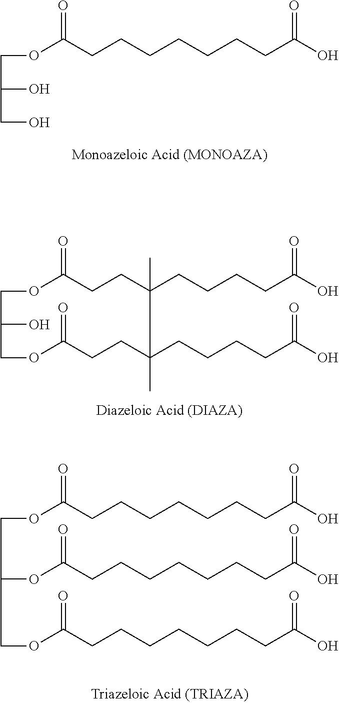 [SMILES: O=C(O)CCCCCCCC(=O)OCC(O)CO ; CC1(CCCCC(=O)O)CCC(=O)OCC(O)COC(=O)CCC1(C)CCCCC(=O)O ; O=C(O)CCCCCCCC(=O)OCC(COC(=O)CCCCCCCC(=O)O)OC(=O)CCCCCCCC(=O)O]

Poly-Aza optional derivative:

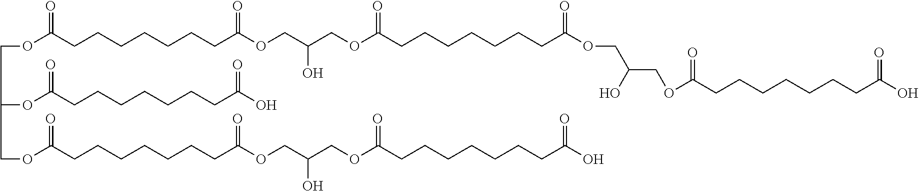 [SMILES: O=C(O)CCCCCCCC(=O)OCC(O)COC(=O)CCCCCCCC(=O)OCC(O)COC(=O)CCCCCCCC(=O)OCC(COC(=O)CCCCCCCC(=O)OCC(O)COC(=O)CCCCCCCC(=O)O)OC(=O)CCCCCCCC(=O)O]

Example of HPLC-SEC chromatogram of representative batch that comprise some MOCL esters of oxidation or/AND “green” synthesis reaction, such as Poly-Aza (polyazelaic glycerol, Mono-Aa-MG-monoglycerol; Di-Aza-DG diglycerol; TriAza −TG triglycerol of azelaic acid and Pelargonic (monocarboxylic nonanoic) acid (FIG. 4):

For the detailed analysis of TriAza, the batches produced by ozonolysis were analyzed by TLC and compared to that synthesized by liquid synthesis. All samples were separated by normal phase chromatography and visualized by either general dye (primulin) or COOH-specific staining (BromCresol Green). It was found that most batches of ozonolysis contain 4-7 clear spots (Rf of the resulting products were identified and compared with known standards). Out of them, 3 major spots were visualized with BromCresol Green, suggesting presence of carboxyl ends. Among them, one spot represents monocarboxylic MOCL nonanoic/pelargonic acid, hence two additional spots might correspond to TriAza molecules. Comparison of Rf of substances obtained by ozonolysis and liquid synthesis has revealed that among the COOH-containing spots two of them are common, i.e., they might represent TriAza. Altogether, we have concluded that using ozonolysis of triolein as intermediate process for “green” synthesis of MOCL, TriAza, pelargonic acid, and its derivatives we succeeded in producing TriAza. General ozonolysis process represented in Scheme 3 below:

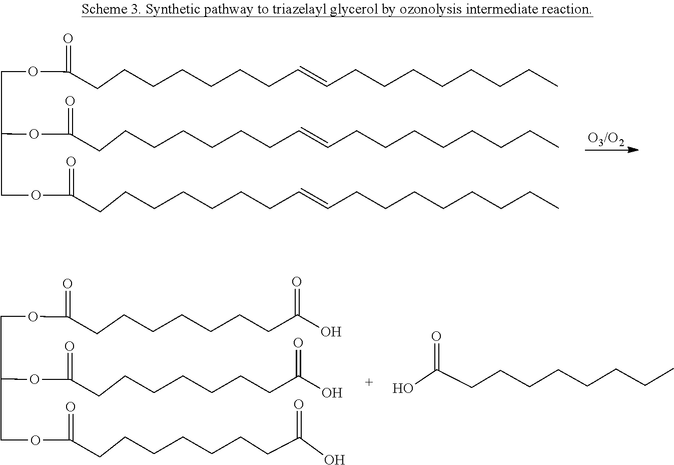

### Example 6: Selection of Process Conditions to Produce TriAza with Green Surfactants

The raw material Olive oil comprises up to 50% oleic acid. Oleic acid is present as free oleic acid, monooleate, di-oleate and tri-oleate. KMno4 added to olive oil cleaves double bond of oleic acid leading to formation of the following MOCL products: pelargonic and azelaic acid (followed by cleavage of oleic acid), Mono-Aza-monoazelate (followed by cleavage of monooleate), Di-Aza-di-azelate (followed by cleavage of di-oleate) and tri-azelate (followed by cleavage of tri-oleate). An additional product of this reaction is MnO-precipitates of black powder. The reaction between olive oil and potassium permanganate under various conditions was performed. The results are summarized in Table 4:

TABLE 7
Results obtained under various conditions in the reaction
between olive oil and potassium permanganate.

| | | | | | | | QC | QC | QC |
|---|---|---|---|---|---|---|---|---|---|
| | | | | | | | Purity: | Identity: | Identity: |
| | | | | Time | | Visual | Trioleate | Pelargonic | TriAza |
| # | Surfactant | Citric | H2O2 | of mix | | Observation | HPLC | GC | MS |
| 1 | − | − | − | 20 | min | Two phases | + | + | + |
| 2 | − | − | − | 1 | h | Two phases | − | + | + |
| 3 | − | + | − | 20 | min | Two phases | + | + | + |
| 4 | − | + | − | 1 | h | Two phases | − | + | + |
| 5 | − | + | + | 20 | min | Two phases | + | + | + |
| 6 | Brij 35 | + | − | 1 | h | One | − | + | + |
| | | | | | | amphiphilic | | | |
| | | | | | | phase with | | | |
| | | | | | | flakes | | | |
| 7 | Sodium cocoyl | + | + | 1 | h | One phase | − | + | + |
| | glutamate | | | | | with flakes | | | |
| 8 | Sodium cocoyl | + | + | 20 | min | Two phases | − | + | + |
| | glutamate | | | | | | | | |
| 9 | Sodium lauroyl | + | + | 20 | min | Two phases | − | + | + |
| | sarcosinate | | | | | | | | |
| 10 | Sodium lauroyl | + | + | 1 | h | One | − | + | + |
| | sarcosinate | | | | | amphiphilic | | | |
| | | | | | | phase with | | | |
| | | | | | | flakes | | | |
| 11 | Sodium cocoyl | + | + | 1 | h | Two phases | + | + | + |
| | glutamate | | | | | | | | |
| 12 | Sodium cocoyl | + | + | 1 | h | One oil | + | + | + |
| | glutamate | | | | | phase | | | |

Examples 7-8: New Green Chemistry Scale Up Industrial Methods of

production of TriAza and related glycerol azeloyl TriAza derivatives. Esters of azelaic acid, especially TriAza, and its derivatives—are the best disclosed ingredients for Olebiome MOCL formula. Previously, we disclosed small scale batches of production of TriAza and evaluated different chemical pathways for the scale-up of triazelaic acid (TRIAZA)-based product, representative processes targets to reach both food and cosmetic markets and thus searches for a chemical pathway as green as possible. The formation of TRIAZA was performed through two different chemical pathways, namely esterification and oxidative cleavage. In the case of esterification of azelaic acid with glycerol, it was shown that the reaction proceeded so easily that azelaic acid tended to polymerize with glycerol, probably forming a hyperbranched polymeric structure.

Example 7: Esterification. Commercially available Azelaic acid (trademark Azepur99®, cosmetic grade) was supplied from Azeco Cosmeceuticals. Glycerol (grade USP) was supplied by Quimicadroga. Both products were characterized by gas chromatography (GC). Purities of 99.5% and 95% were observed for both Glycerol and Azelaic Acid respectively.

Half kg batch L24-0024 production: Two reactions were performed in a 500 mL three-neck round bottom flask into which both azelaic acid and glycerol were added with the ratios described in Table 5 below.

TABLE 5
Reaction parameters involved in the lab-scale esterification trials

| | | Batch #1 | Batch #2 |
|---|---|---|---|
| | | (L24-0003) | (L24-0007) |
| Azelaic Acid (AZA) | Mass (g) | 200.2 | 201 |
| | Moles (mol) | 1.06 | 1.06 |
| Glycerol | Mass (g) | 33.5 | 16.9 |
| | Moles (mol) | 0.35 | 0.18 |
| Ratio Glycerol / AZA | mol/mol | 1/3 | 1/6 |
| Temperature | ° C. | 160° C. | From 120° C. to 170° C. |
| Yield | % _{w/w} | n.d. | 52% |

n.d.: not determined

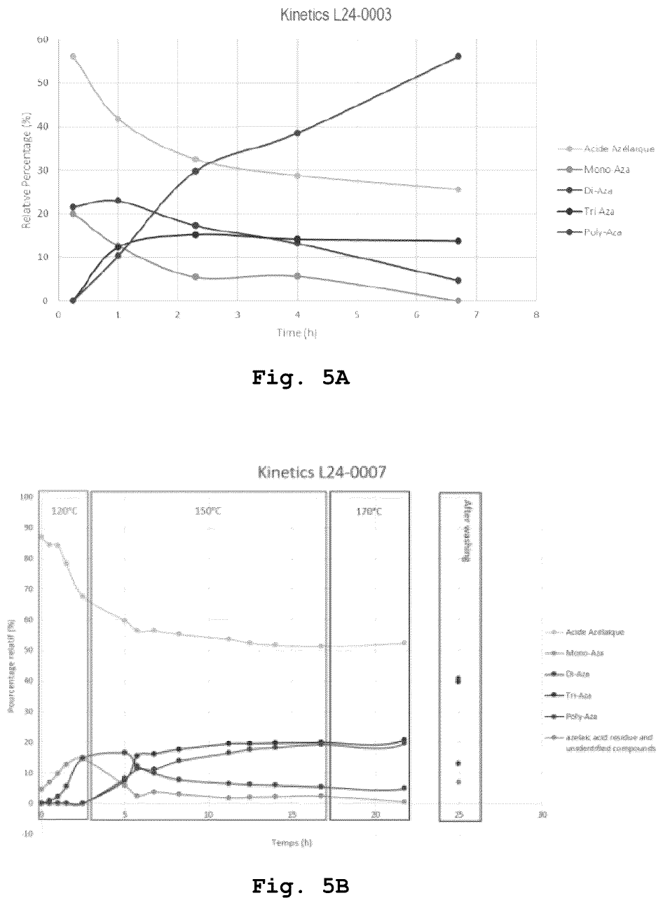

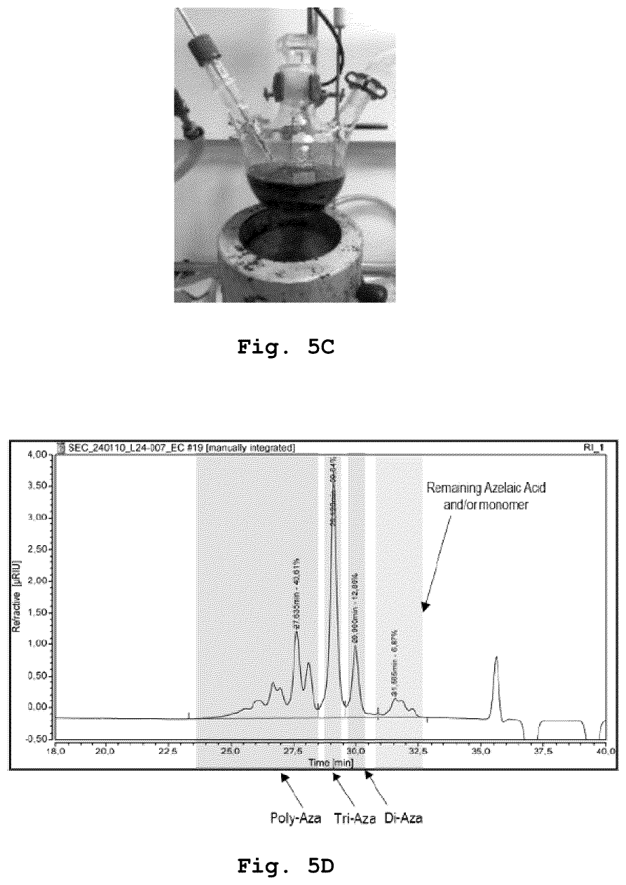

The azelaic acid being solid, it was first melted before glycerol is added onto it. The reactions were heated at temperatures higher than 100° C. so that the generated water by-product evaporates. The reactions were followed by HPLC-SEC and the results obtained are depicted in FIGS. 5 (A-D). SEC allows for the identification and characterization of polymeric Azeloyl glycerol-TriAza derivatives products. A temperature of 120° C. (controlled heating) was thus set to allow for the evaporation of water. After 2.5 h of reaction, Mono-AZA and Di-AZA-TriAza derivatives were formed. The temperature was thus increased to 150° C. during 15 h and it could be observed that TRIAZA got formed as well as polymers while the relative concentrations of Mono-AZA and Di-AZA decreased. Washing with hot water was performed on the melted final mixture. Azelaic acid being poorly soluble in water, extensive washing steps were necessary to obtain 61.2 g of a brownish liquid product (yield of 53% w/w). It was composed of 40% of TriAza, 40% of polymers and 13% of Di-AZA, the rest being remaining AZA and unidentified products of low molar masses. FIG. 5A and FIG. 5B demonstrate HPLC-SEC follow-up of L24-0003 and L24-0007 respectively. FIG. 5C shows a picture of the reaction set-up of L24-0007 (TriAza batch) and FIG. 5D shows an HPLC-SEC trace obtained during the synthesis of L24-0007 (TriAza batch), indicating the final composition.

Kilolab-scale esterification trials—the synthesis of batch L24-0024.

The synthesis of TRIAZA was then tried on a kilo-lab scale considering the results obtained at the lab-scale (namely an increased ratio of Glycerol/AZA and a low reaction temperature). Azelaic acid (trademark Azepur99®, cosmetic grade) supplied from Azeco Cosmeceuticals was used for these reactions and put to react with glycerol in ratios described in Table 6. To avoid any potential oxidation, the reaction was performed under controlled nitrogen atmosphere.

TABLE 6
Reaction in process QC- quality control parameters involved in the kilolab-scale esterification trials

| | | Batch #3 | Batch #4 |
|---|---|---|---|
| | | (L24-0024) | (L24-0040) |
| Azelaic Acid (AZA) | Mass (g) | 502 | 2215.1 |
| | Moles (mol) | 2.66 | 11.69 |
| Glycerol | Mass (g) | 40.8 | 107.7 |
| | Moles (mol) | 0.44 | 1.17 |
| Ratio Glycerol / AZA | mol / mol | 1/6 | 1/10 |
| Temperature | ° C. | From 120° C. to 110° C. | 110°C |
| Yield | % _{w/w} | 22% | 17% |

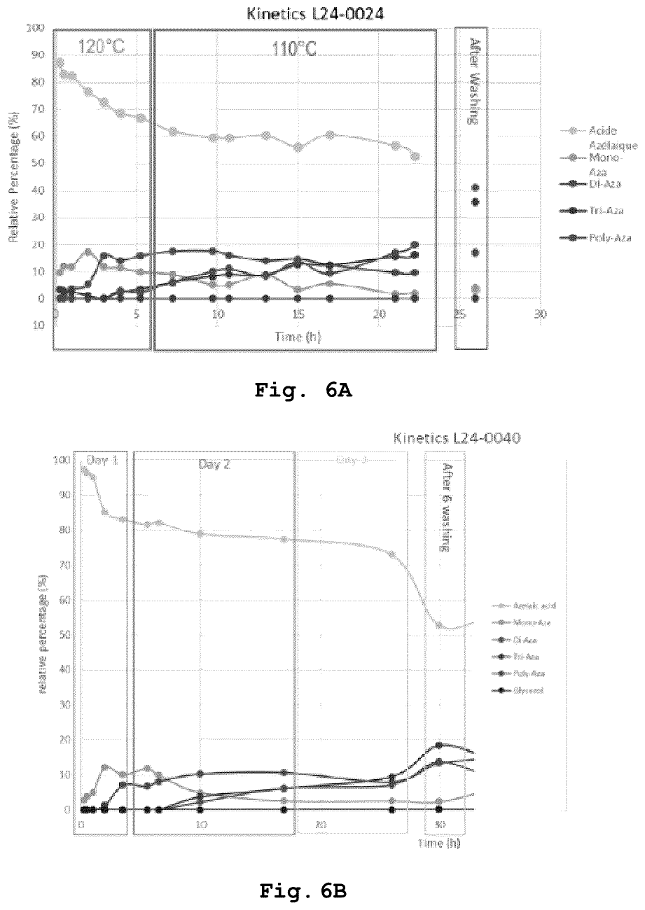

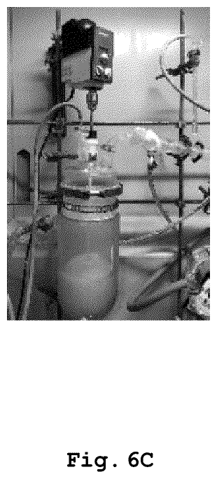

The esterification conditions were kept similar to L24-0007, except for the temperature that was kept at 120° C. from the beginning of the reaction. FIG. 6A and FIG. 6B demonstrate HPLC-SEC follow-up of L24-0024 and L24-0040 TriAza MOCL batches, respectively. FIG. 6C shows a picture of the reaction set-up of L24-0040.

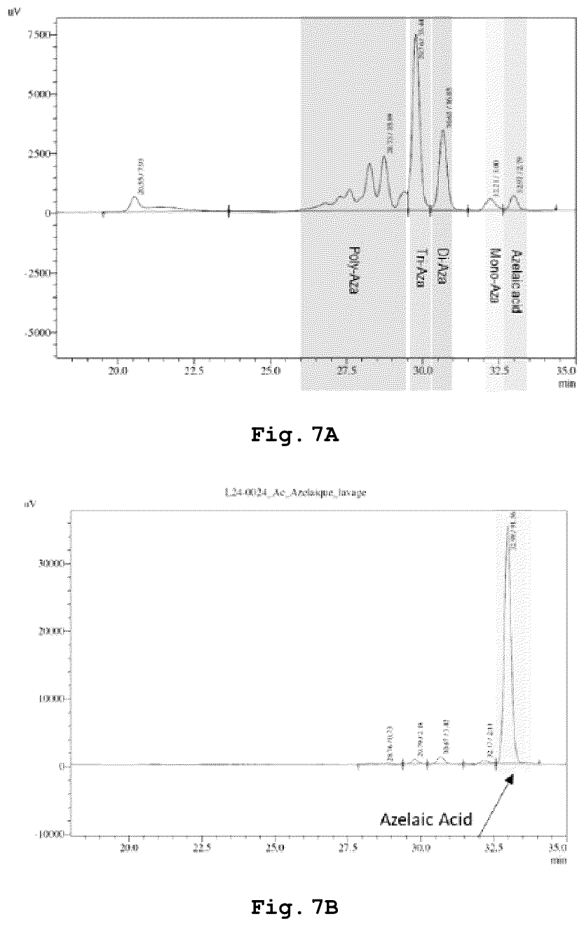

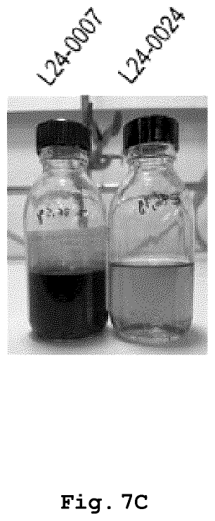

In these conditions, only Mono-AZA and Di-AZA were formed at the beginning of the reaction. Pure AZA was regularly consumed. However, after 3 h of reaction, slight amounts of TRIAZA and polymers were formed (relative concentration <5% w). To avoid more polymer to form, the reaction temperature was decreased down to 110° C. and kept constant during 16.5 h. A similar work-up procedure, employing hot water was used and 16 washing steps were necessary to reach a pH of 5 in the water phase (after washing) and a remaining AZA concentration of 3% in the obtained yellow oil phase as shown in FIG. 7 (A-C). The rest of the product was composed of 3% Mono-AZA, 17% of Di-AZA, 33% of TRIAZA and 40% of polymers. FIG. 7A demonstrates HPLC-SEC trace of L24-0024 after washing with hot water. FIG. 7B demonstrates an HPLC-SEC trace of collected AZA from the washing aqueous phases, and FIG. 7C shows a picture of a comparison between the obtained products.

### Example 8. The Oxidative Cleavage of Dihydroxylated VHOSO (diOH VHOSO) Using Bleach as Oxidant. Synthesis of TRIAZA and its Derivatives by Dihydroxylation of Epoxides

Very High Oleic Sunflower Oil (VHOSO) was selected for this pathway. Epoxidized VHOSO was prepared on site and used straightly for the dihydroxylation process. In an additional experiment epoxidized Olive oil with 73% of triolein was utilized. In additional scale up experiment oxidation of ozonated VHOSO an olive oil was used as precursors for cleavage of double bonds. The dihydroxylation process relies on the ring-opening of the epoxy moiety by reaction with water in acidic conditions, as depicted in Scheme 6.

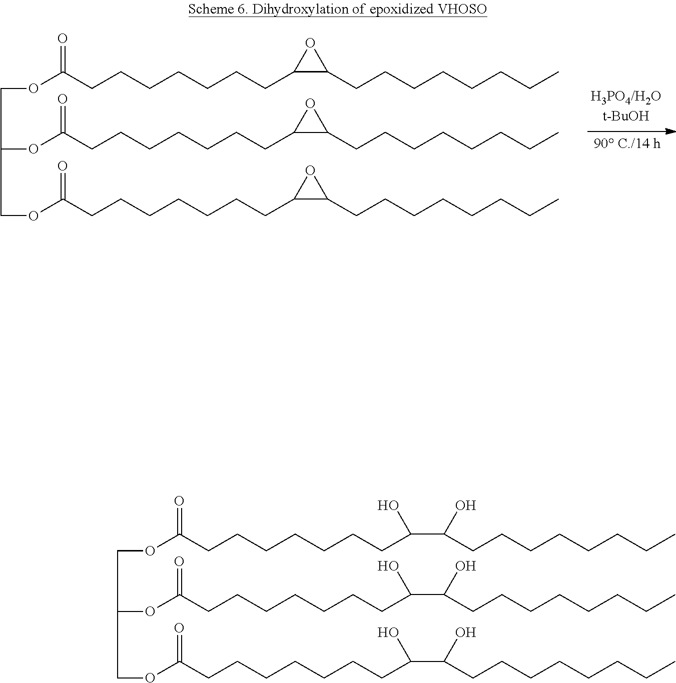 [SMILES: CCCCCCCCC1OC1CCCCCCCC(=O)OCC(COC(=O)CCCCCCCC1OC1CCCCCCCC)OC(=O)CCCCCCCC1OC1CCCCCCCC ; CCCCCCCCC(O)C(O)CCCCCCCC(=O)OCC(COC(=O)CCCCCCCC(O)C(O)CCCCCCCC)OC(=O)CCCCCCCC(O)C(O)CCCCCCCC]

The tert-Butanol green solvent1 was also added into the mixture to help fluidize the reactive mixture upon formation of the diol derivative. Two batches of dehydroxylated VHOSO were synthesized: L24-0073 and L24-0115. The reactions were performed in a 4L-reactor into which 2 kg of VHOSO were added, as well as tert-Butanol (⅓ of oil's weight). The obtained oils were methylated so that a mixture of fatty acid methyl esters (FAMEs) representing the oil composition is formed. The cocktail of FAMEs was then silylated prior to injection in the GC equipment. The obtained oils were silylated and directly injected in the GC equipment so that the composition in fatty acids, mono-, di- and triglycerides (MG/DG/TG) is known.

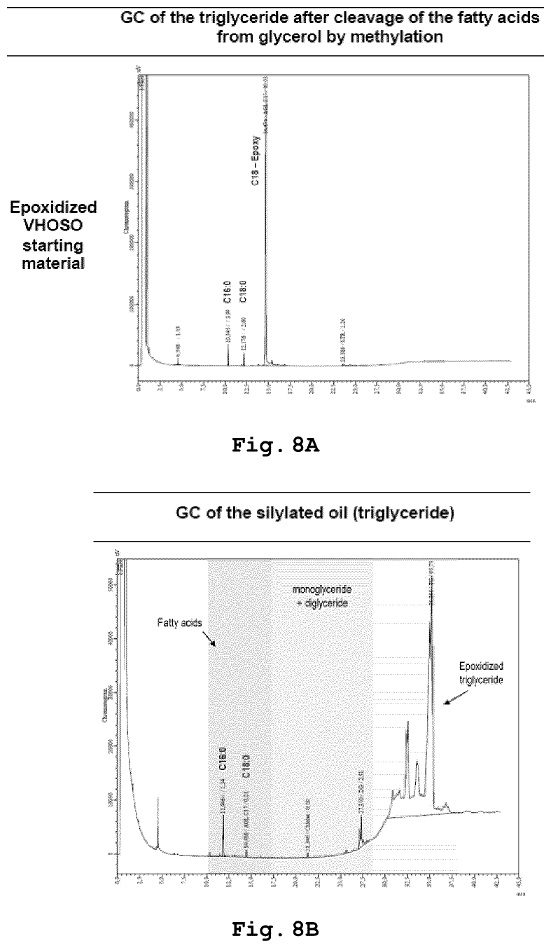

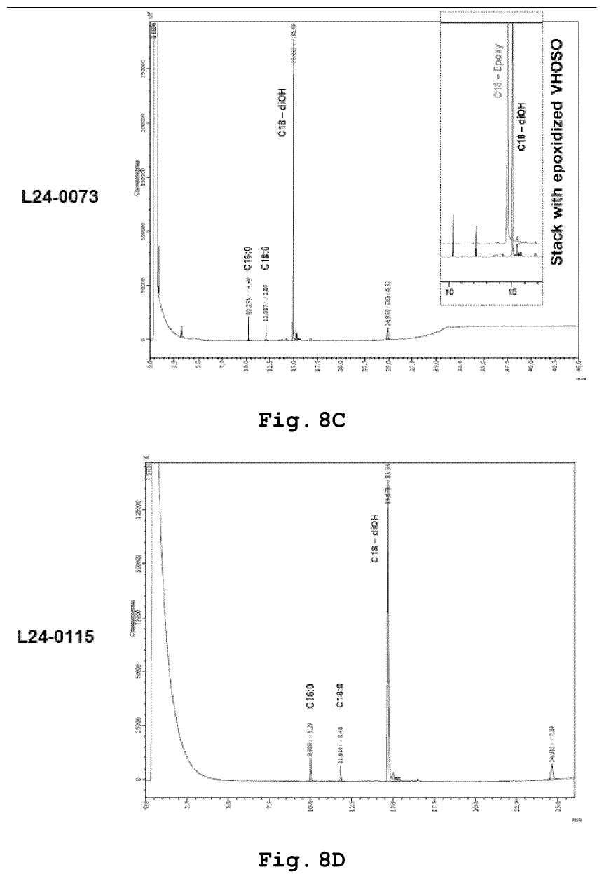

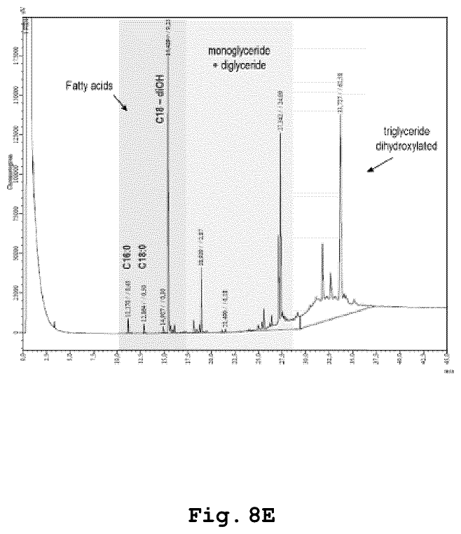

The methylated samples confirmed that all the epoxides moieties reacted during the ring opening, as depicted in FIG. 8C, with more than 85% of the fatty chains being epoxidized oleic acid (in the case of the starting material, FIGS. 8A-B or dihydroxylated oleic acid (in the cases of batches L24-0073 and L24-0115).

FIGS. 8A-E show the GC chromatograms obtained for the starting material (epoxidized VHOSO), as depicted in FIGS. 8A-B and for the two batches of dihydroxylated VHOSO (L24-0073, as depicted in FIG. 8C and L24-0115, as depicted in FIGS. 8D-E. Two types of sample preparation were made for the analysis:

The obtained oils were methylated so that a mixture of fatty acid methyl esters (FAMEs) representing the oil composition is got. The cocktail of FAMEs was then silylated prior to injection in the GC equipment.

The obtained oils were silylated and directly injected in the GC equipment so that the composition in fatty acids, mono-, di- and tri-glycerides (MG/DG/TG) is known.

TABLE 7
Compositions in Fatty acids, MG, DG and TG of
Epoxidized VHOSO and L24-0115 (dihydroxylated VHOSO) as obtained
by GC analyses after silylation of the samples. The results are
provided in % wt.

| | | Epoxidized | |
|---|---|---|---|
| | | VHOSO | L24-0115 |
| Fatty Acids | C16:0 | 1.3% | 0.5% |
| | C18:0 | 0.2% | 0.3% |
| | C18-diOH | — | 9.2% |
| | Others | — | 0.3% |
| Monoglycerides + Diglycerides | — | 2.7% | 27.1% |
| Triglycerides | — | 95.8% | 62.8% |

Indeed, the percentages of free fatty acids, MG and DG went from 4.2% wt to 37.4% wt. This chemical pathway indeed proved to be suitable for fatty acid methyl ester derivatives and is compliant with Green Chemistry, as shown in Scheme 7.

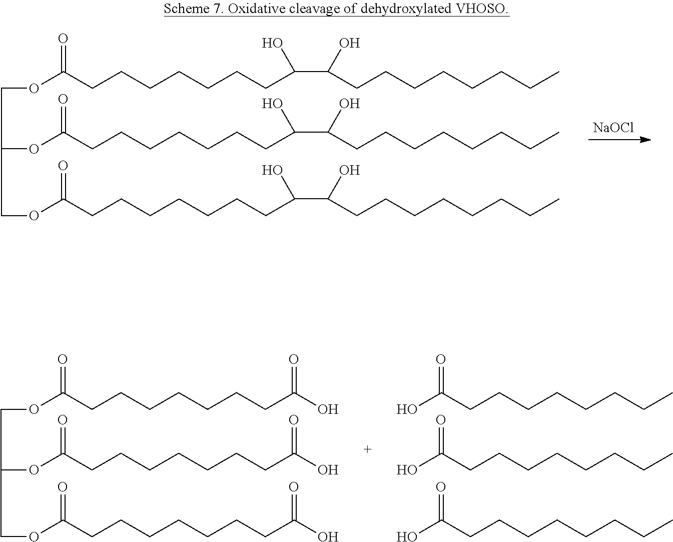

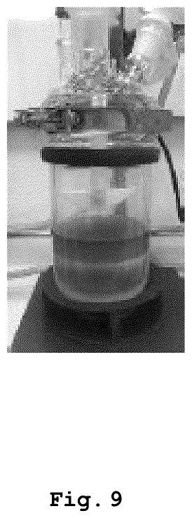

Bleach was poured into a 1L-glass reactor equipped with mechanical stirring, a temperature reader and a condenser, as depicted in FIG. 9. diOH VHOSO was melted in the oven prior to being added into the reactor. No heating was provided to the reaction. A very strong exothermic reaction, which reached a temperature of 107° C. in 5 minutes before it started to cool down was noted. It took 4 h of reaction time for the temperature to get down to 26° C. When the reaction was over, the aqueous phase (bleach) separated easily on top of the yellowish oil phase by simple decantation and could be separated, as depicted in FIG. 9.

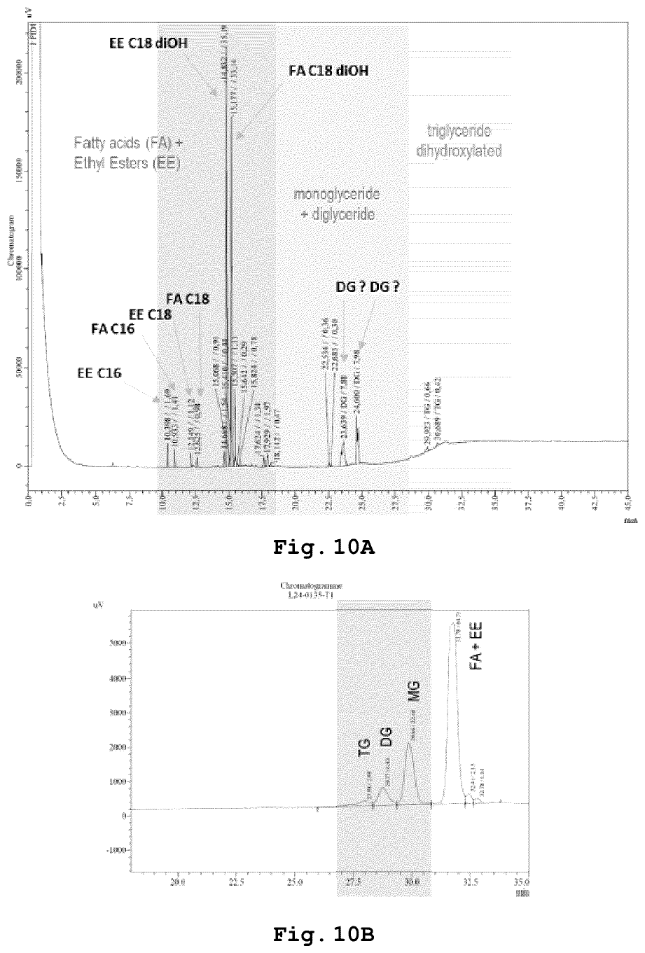

Batch L24-0135, diOH VHOSO and Ethanol were added into a 2L-reactor. diOH VHOSO solubilized directly into the solvent. Bleach was then added in 1 h to the reaction mixture under mechanical stirring. No exothermic behavior was noticed (the maximum temperature reached was 32° C.). The reaction mixture was then left to phase separate and bleach being the bottom phase, it could be easily separated. The reaction mixture was then washed with an acidic aqueous phase and again the mixture tended to emulsify, making the separation process complicated. 72 g of product were collected (yield of 48% wt/wt) and it was analyzed by HPLC-SEC and GC as shown in FIG. 10A-B, respectively and in Table 8.

TABLE 8
Composition in Free Fatty Acids, MG, DG and TG from

| | | % _{wt} | % _{wt} |
|---|---|---|---|
| | | at t₀ | at t_{final} |
| Fatty Acids | C16:0 | 0.5% | 3.1% |
| | C18:0 | 0.3% | 2.1% |
| | C18-diOH | 9.2% | 68.3% |
| | Others | 0.3% | 3.5% |
| Monoglycerides + | — | 27.1% | 16.5% |
| Diglycerides | | | |
| Triglycerides | — | 62.6% | 1.1% |

L24-0135 as obtained by GC analyses. The results are provided in % wt.

No polymer was noticed. The GC however showed that almost no triglyceride remained in the final mixture whereas the concentration of products eluting at retention times corresponding to fatty acids (and their ester forms) dramatically increased (77%), the rest mostly being MGs and DGS. A deeper analysis of the GC chromatograms also showed that ethyl esters had appeared. This was surprising since no heat was provided to the mixture. It may be that NaoCl being alkaline, catalyzed the transesterification of TGs to yield ethyl esters in addition to fatty acids that appeared by hydrolysis.

It was thus concluded that EtOH was not a suitable candidate as a solvent for this process because of its nucleophilicity making it prone to be engaged in transesterification reactions. For this reason, tert-Butanol was selected instead. It is indeed a green alcohol that does not exhibit any nucleophilic character.

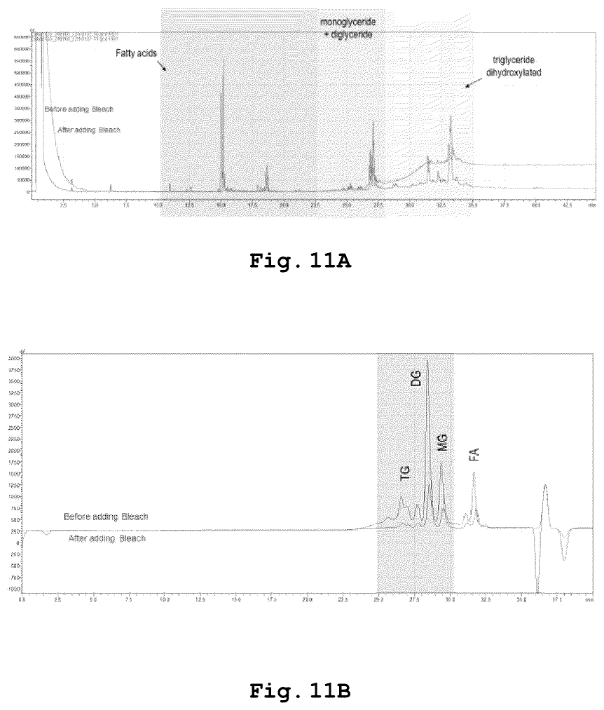

Batch L24-0137-diOH VHOSO and tert-Butanol was run in the exact same conditions of L24-0135, excepted for the solvent. L24-0137 was run in the exact same conditions as L24-0135, excepted for the solvent. Notably, diOH VHOSO is not fully soluble in t-BuOH at a concentration of 1 g/4 mL when added at room temperature. It became soluble after being heated at 50° C. in this solvent and brought back to RT. The addition of bleach was then performed in 15 min without any exothermic behavior noticed (the temperature plateaued at 20-21° C.). From the GC and HPLC-SEC analyses of the resulting mixture shown in FIG. 11A-B, respectively, it can be noticed that no difference is observed, meaning that no hydrolysis nor polymerization occurred. Notably, diOH VHOSO is not fully soluble in t-BuOH at a concentration of 1 g/4 mL when added at room temperature. It got soluble after being heated at 50° C. in this solvent and brought back to RT. The addition of bleach was then performed in 15 min without any exothermic behavior noticed (the temperature plateaued at 20-21° C.). From the GC and HPLC-SEC analyses of the resulting mixture shown in FIG. 11A-B, it can be noticed that no difference is observed, meaning that no hydrolysis nor polymerization occurred.

### Example 9: Microbiome Balance Effect: Microbial Lipase Targeting Mediated Activity of Anaplerotic TriAza −Glycerol Ester of Dicarboxylic Nonanoic MOCL

The activity of C. acnes-associated and P. acnes-associated lipase was tested in the presence of triolein (a trigger of lipase activity). Bacteria was grown for 4 days in liquid medium. P. acnes after double passage on Chocolate plates (Novamed) were grown bacteria in anaerobic conditions at 37° C. for 1-2 days. The bacteria were Gram stained, and the proper morphology of the bacteria was assured. Then, 1 O.D. 600 bacteria were resuspended in 1 ml of Thioglycolate medium (HyLabs) containing 50 μl triolein and grown in anaerobic conditions at 37° C. for 4 days. Following this incubation, 100 μl of emulsified substrate was added to the culture that was further maintained at 37° C. for overnight. Afterwards, the supernatant was separated from the bacteria by 1 min centrifugation at 10000 rpm and subjected to lipid extraction using clorophorm: methanol (2:1). The lipids after nitrogen evaporation and resuspension in clorophorm: methanol (2:1) were separated on silica TLC plates in a development solution of clorophorm: methanol: water (65:25:4), and visualized with BromCresol Green (Sigma).

### Example 10: Microbiome Balance Effect: Triolein is a Sufficient Substrate for Lipase Producing Microorganisms on Example of Staph. Aureus, Proving Use of Triolein Enriched Composition as Prebiotic for Lipophilic Microbiome

S. aureus bacteria was plated on the solid agar Petri dishes (1) nutrient free, (2) agar with spot with emulsified Triolein, and (3) plate with spot with emulsifier only. On the plates bacteria were incubated at 37° C. for 2 days. S. aureus growth was tested using colorimetric assay in the presence of emulsified triolein solid medium agar culture as shown in FIG. 12.

Results:

It appears that triglyceride (TG) is a sufficient and essential nutrient for skin microbiome, as shown in the plate containing emulsified triolein which allowed bacterial colonization, compared to lack of growth in the negative control or the plate containing only the emulsifier (FIG. 12). It is concluded that lipase-mediated hydrolysis of triolein generates free fatty acid as an essential energy source for growth of S. aureus.

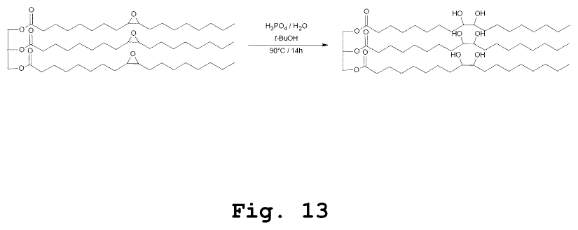

Example 11: The controlled “green” chemistry by dihydroxylation process of epoxidized Triolein-enriched oil. The dihydroxylation process relies on the ring-opening of the epoxy moiety by reaction with water in acidic conditions, as depicted in FIG. 13. The tert-Butanol green solvent1 is also added into the mixture to help fluidize the reactive mixture upon formation of the diol derivative. Very High Oleic Sunflower Oil (VHOSO) was selected as an interesting candidate for this pathway. VHOSO is known to be composed of about 90% of oleic acid (C18: 1), making it a very interesting candidate for the synthesis of TRIAZA since it would in theory maximize the formation of TRIAZA and pelargonic acid other instead of by-products. Moreover, epoxidized VHOSO was used for generation of MOCL TriAza derivatives.

Example 12: Microbiome balance effect: Selection of methods for QC-quality control-Functional Potency. Testing minimal inhibitory concentration (MIC) to P. acnes was executed by microdilution method. TriAza based biocontrol, microbial preservation. P. acnes after double passage on Chocolate plates (Novamed) were grown bacteria in anaerobic conditions at 37° C. for 1-2 days. The bacteria were Gram stained and the proper morphology of the bacteria was assured. Then, bacteria were resuspended in Thioglycolate medium (HyLabs) at concentration of 0.02 O.D.600. Meanwhile, ELISA plates containing at the first raw 200 μl of undiluted tested material were prepared. The rest wells were filled with 100 μl of Thioglycolate medium and 11 serial 1 dilutions by transferring 100 μl of undiluted material was performed. Once the ELISA plate was ready, 100 μl of prepared bacterial culture was added. Bacteria alone and medium w/o bacteria were used as standard controls. The plates were incubated in anaerobic conditions at 37° C. for 2 days. Minimal inhibitory concentration was determined visually and spectrophotometrically, while minimal bactericidal concentration was determined after plating of the bacteria on solid medium (Chocolate plates). On the plates bacteria were incubated in anaerobic conditions at 37° C. for 2 days and the number of appearing colonies were counted manually. Specification for positive functional potency for batch acceptance for the TriAza batches tested are selected in range of antimicrobial activity-MIC between 0,25-1 mM.

### Example 13: MOCL-Olebiome Anaplerotic Composition Comprising TriAza Mixtures Generation

Odd numbered fatty acids are found in small amounts acylated to various sphingolipids where they have unique properties and functions. Phospholipids of azelaic acid, cerebrosides, sphingolipids as natural metabolites of odd chain fatty acids are described in prior art.

No use of external compositions of mixtures comprising triglycerides, diglycerides, monoglycerides and mixtures enriched with triacylglycerols of azelaic acid and other C9 OCFA for use in longevity multifunctional food and topical formulations were described before.

Suprisingly, the disclosed composition can be enriched with the precursors of odd chain fatty acids. OCFA can endogenously bio-synthetized from precursors short chain FAS (e.g. tripropionate, propionic acid, succinate and derivativs of thereof) to long chain OCFA (C15; C17-ceramides) by internal elongases and other enzymatic machinery as in mammalian host and also, putatively, with help of microbiome.

TRiAza multifunctional glycerol conjugates or C9 MOCT/OCFA mixtures may comprise additional precursors or/and derivatives of OCFA (as exemplified in Table 9 below) and also derivatives, such as esters, dicarboxylic (e.g. brassylic acid), conjugated, hydroxy and branched isomers and glycerol's. Intracellular fatty acid elongase enzymes generate C9 OCFA from OCFA precursor with C 3-7 and derivative of OCFA can be derived/generated by elongases from C9 towards C11; C13 (brassylic), C15, C17 etc. up to very long chain saturated-OCFA (Table 9A). Commercialy available today C9 derivatives linear and branched are substrate for esterases and will form C9 FA post hydrolytic cleavage by intacellular esterase/hydrolase enzymes, same relevant for diesters of azelaic acid dicarboxylic precursors and diester derivatives (Table 9B).

TABLE 9
Optional Oblebiome anaplerotic composition, comprising precursors
or/and derivatives of MOCL

| A. C9 FA Precursors | |
|---|---|
| and Derivatives/ | B. Derivatives/precursorslinear |
| Carbon number | and branced nonanoates esters, |
| Systematic Name | diesters of C9 FA |
| C3-Propanoic acid | ISONONYL ISONONANOATE |
| C5-Pentanoic acid | CETEARYL NONANOATE |
| C7-Heptanoic acid | ETHYLHEXYL PELARGONATE |
| C9-Nonanoic acid | PROPANEDIOL DIPELARGONATE |
| C11-Undecanoic acid | CETYL STEARYL ISONONANOATE |
| C13-Tridecanoic acid | NEOPENTYL GLYCOL |
| | DIISONONANOATE |
| C15-Pentadecanoic acid | PENTAERYTHRITYL |
| | TETRAPELARGONATE |
| C17-Heptadecanoic acid | DIETHYL AZELATE |

TriAza can be obtained by oxidation of vegetable oils or animal fats as raw material enriched with triglycerides (TGs), or purified TG that comprise fatty acid-FA with n-9 (omega-9) unsaturation bond, or TG of C20 (n-11), whereas during oxidative cleavage, essentially at acidic QC-controlled conditions, i.e. without hydrolysis, TriAza examples of relevant FAs depicted below:

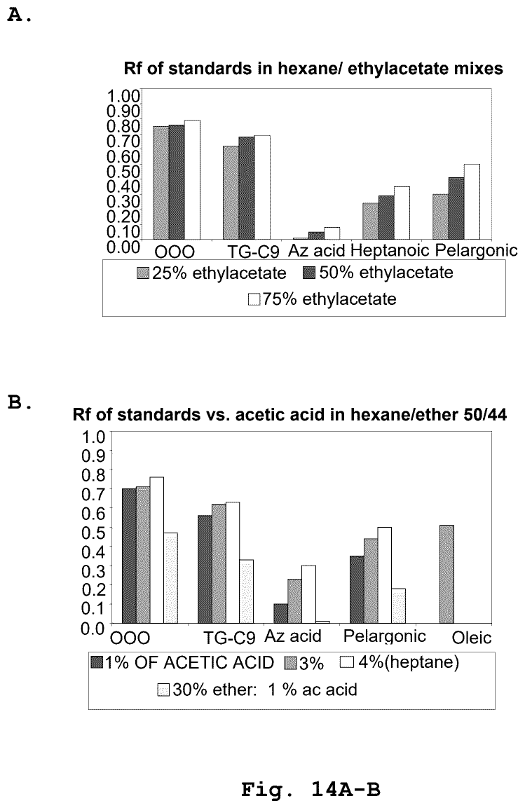

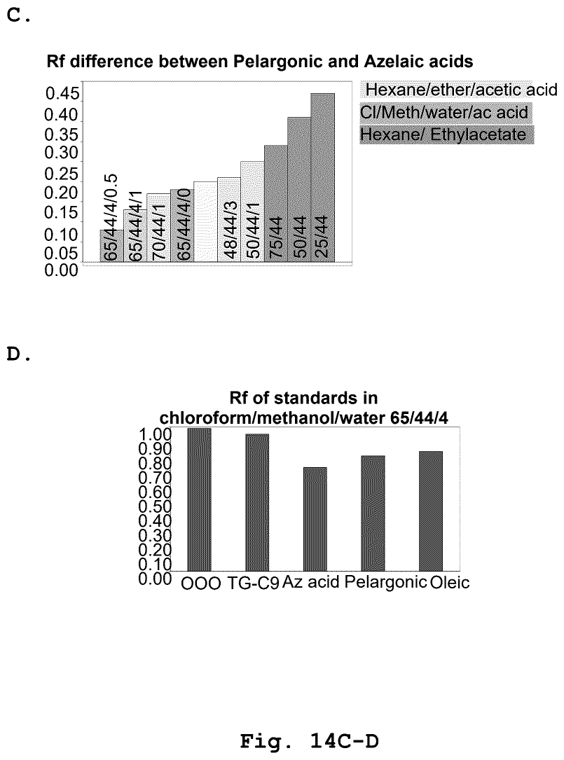

Analytical QC characterization. Thin layer chromatography (TLC) assays were tested with different running solvents for better separation of pair triacylglycerols-FFA and FFA-dicarboxylic acids. The best results were obtained with primulin. Bromcresol green was chosen for distinction between triglycerides and fatty acids: 0.05-1% solution Rhodamine B in ethanol or 2% solution in water following with spraying 10N KOH; 0.01% (w/v) primulin solution in acetone-water (60:40, v/v) with UV detection; 0.2% 2,7-dichlorofluorescein in methanol; 0.3 g Bromcresol green in mix Methanol/water (80° ml/200) with 8 drops of 30% NaOH; Sensitivity of coloring: about 1 μl of 100 mM solutions. Representative results of qualitative and quantitative control analytical results, represented in FIG. 14, characterize relative recovery of C9 Triaza derivatives of ozonolysis reaction including: 000-ozonides, AzaA-azelaic acid, Pelargonic acid/PA, and TriAza −TG9-derivatives.

### Results:

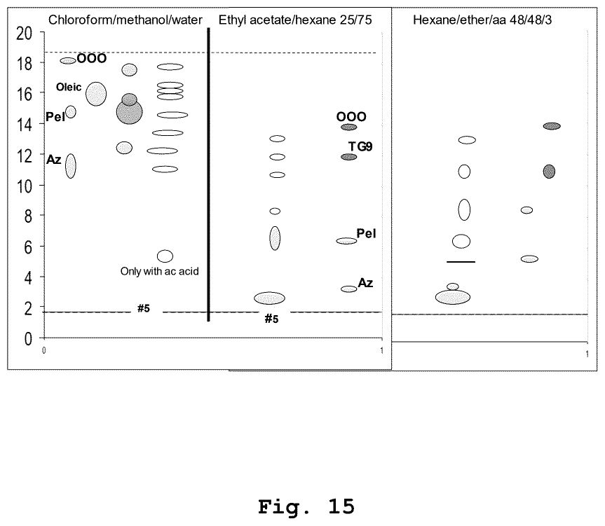

TLC spot distributionwere analysed in different feeding acid and methods and selected based on chloroform/methanol/water best separation ability. The chromatography, shown in FIG. 15 indicates that ozonolysis feed method with formic acid results (grey spots) in three end products of the reaction only: TriAza −TG9-(proven by MS and NMR analysis), PA-pelargonic by product and single ozonide band as non-utilized raw material. Esterification method-liquid synthesis resulted in highest yield of TriAza and di-Aza as by product, with underutilized azelaic acid (utilized in oversaturation) and DMAP reagent as by products. TriAza mix contained TriAza and Pelargonic acid recovered for MS analysis and chromatography by various analytical methods at various ratio as shown below.

MS were measured by LC/MS (Finnigan LCQ™ DUO, USA) in different modes. The LCQDUO MS detector uses quadrupole ion trap mass analyzer with an ion source external to the mass analyzer. All data are presented in Table 10 below:

TABLE 10
MS-MS Mass-spectroscopy analytical results.

| | |
|---|---|
| I | 1081 |

| OOO | 885.4 | | 886.4 | 885.3 | | | | | | | 885.3 |
|---|---|---|---|---|---|---|---|---|---|---|---|
| G | | 778 | | | 773.4 | | 773.5 | | 778.4 | | 778.5 |
| F | | 759 | 756 | 757.2 | 759.4 | | 759.4 | | | | 759.4 |
| E | | | | | 742.6 | | 745.5 | | 745.4 | | |
| TRIAZA | 602.7 | | | | 602 | 603 | 602 | 603 | 603.5 | 604.5 | 603.5 |

| | | | | | | |
|---|---|---|---|---|---|---|
| D | 586 | 581.3 | 584.5 | 585.5 | 585.6 | 585.7 |

| | | | | | | | | |
|---|---|---|---|---|---|---|---|---|
| C | 570 | 564.9 | 559 | 572 | 570 | 572 | 569.7 | 569.7 |

| B | | 556 | | | 555.6 | 555.7 | 553.6 | | |
|---|---|---|---|---|---|---|---|---|---|
| DiAza | 432.5 | | 443.2 | 431.5 | 431.6 | | | | |
| A | | 416 | | 415.4 | 415.5 | 415.5 | 415 | 416 | 417 |
| MonoAza | 262.3 | | | | 261.4 | | | | |

| | | | | |
|---|---|---|---|---|
| Azel | 188.2 | 188.4 | 187.3 | 187.3 |

| | | | | | |
|---|---|---|---|---|---|
| Pelargonic | 158.24 | 157.1 | 157.3 | 157.3 | 177.3 |

| | | | | | |
|---|---|---|---|---|---|
| 1.21 E3 | 4.14 E2 | 3.12 E4 | 6.28 E4 | 4.33 E5 | 9.23 E4 |
| AV 309 | AV 13 | AV 311 | AV 479 | AV 30 | AV 30 |
| 0.02-4.93 | 0.78-1.02 | 0.02-5.01 | 0.03-7.73 | 0.59-1.07 | 1.16-1.66 |
| −c | −p | −c | −c | +p | +p |

| | | | | | |
|---|---|---|---|---|---|
| Batch | OOO | OOO | #1 | #2 | #3 |

| I | | | | | | | | | | | |
|---|---|---|---|---|---|---|---|---|---|---|---|
| OOO | 885.4 | | | 889.1 | | | | | | 881.8 | |
| G | | | | | | 778.3 | | | | | |
| F | | | | 761.3 | | | | | | 759.3 | |
| E | | 731.3 | | 747.3 | | 743.5 | | | | | |
| TRIAZA | 602.7 | 603.5 | | 603.5 | 604.5 | | | | | 601.6 | 601.7 |
| D | | 585.6 | | 585.6 | | | | | | 585.5 | 585.6 |
| C | | 569.7 | | 569.7 | | 569.8 | | | | | 569.5 |
| B | | 555.8 | | 555.8 | | 555.9 | | | | | |
| DiAza | 432.5 | | | 429.6 | | 429.4 | | | | | 431.1 |
| A | | 416 | 416 | 415.6 | | 415.7 | | | | | |
| MonoAza | 262.3 | 257.5 | | 259.3 | | 258 | 259 | 260 | 261 | | |
| Azel | 188.2 | | | 187.3 | | | | | | | |
| Pelargonic | 158.24 | | | | | | | | | | |

| | | | |
|---|---|---|---|
| 3.14 E5 | 2.77 E5 | 3.57E+03 | 1.33E+03 |
| AV 48 | AV 27 | AV19 | AV 24 |

| | | | | |
|---|---|---|---|---|
| 0.22-0.99 | 0.21-0.64 | 0.53-1.62 | 0.26-0.66 | 0.49-0.93 |
| +p | +p | +p | −p | −p |

| | | | |
|---|---|---|---|
| Batch | #4 | #5 | #6 |

| I | | 1203 | 1226.4 | | 1204.1 | 1203.7 | | 1203.7 |
|---|---|---|---|---|---|---|---|---|
| OOO | 885.4 | 988 | | | | | | 884.1 |
| G | | | | | | | | |
| F | | 757.9 | | | 758.1 | | | |
| E | | | 744 | 744.9 | | 743.6 | | 743.8 |
| TRIAZA | 602.7 | 601.9 | 602.4 | | 602.6 | 602 | 604.3 | 602.5 |
| D | | | 586.3 | | 588.5 | 588.7 | | 588.6 |
| C | | | 574.1 | | 574.3 | 574.6 | | 574.5 |
| B | | 558 | | | | | | |
| DiAza | 432.5 | 431.8 | 431.9 | | | 432.2 | | 432 |
| A | | | | | 413.9 | 430.7 | | |
| MonoAza | 262.3 | 261.6 | 263.2 | | 263.5 | 261.9 | | 261.4 |
| Azel | 188.2 | 187.4 | 187.6 | | 189.1 | 187.8 | | 187.7 |
| Pelargonic | 158.24 | | | | | | | |

| | | | | |
|---|---|---|---|---|
| 5.91 E4 | 6.28 E4 | 6.28 E4 | 1.59 E4 | 1.39 E4 |
| AV 180 | AV 479 | AV 479 | AV 8 | |
| 2.06-5.85 | 2.2-3.2 | 0.88-1.92 | 1.11-1.38 | 1.11-2.47 |
| −p | −p | −p | −p | AV 34 |

| | | | | | |
|---|---|---|---|---|---|
| Batch | O19 | 1 hour | O19 | 3 hour | O18 |

Besides of triolein, diglycerols of oleic and linoleic acids also produced Mm similar to TriAza by positive-ion APCI mass spectra (Scheme 5) and dioleyl glycerol produce characteristic ions with mass 603.6. Additional, mass spectra of triolein oxidation products included hydroperoxides, epoxides and ketones, analyzed by atmospheric pressure chemical ionization (APCI) mass spectrometric detection, produce Mm 601.6-603.6 for all samples.

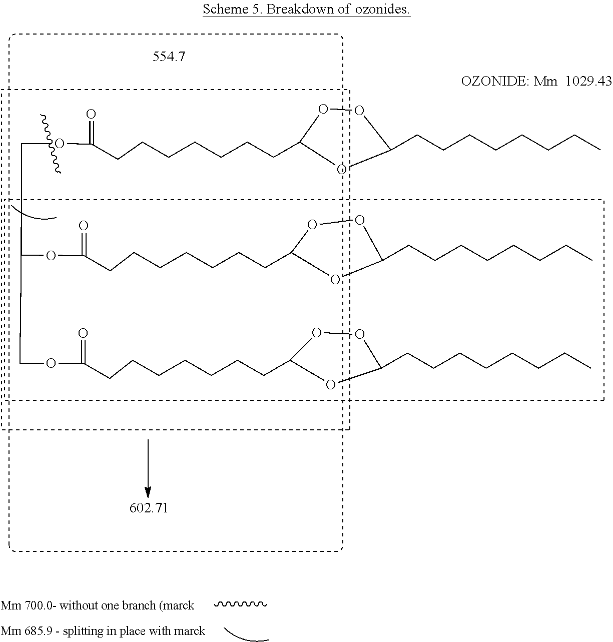

All spectra were compared to each other and the representative peaks were emphasized and summarized in Table 11 below:

TABLE 11
Mass spectrometry observed for various substances

| Substance | Mm | Mm observed | Possible structure of MOCL TriAza derivatives |
|---|---|---|---|
| Pelargonic | 158.24 | 157.3 | |
| C9-nonanoic | | | |
| Azelaic | 188.22 | 187.3 | |
| C9-dicarboxylic | | | |
| Mono-glycerol-Aza | 262.3 | 261.6 | |
| A- diglycerol C9 derivative 1 | 416.5 | 415.5 | 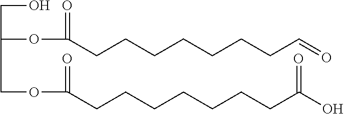 [SMILES: O=CCCCCCCCC(=O)OC(CO)COC(=O)CCCCCCCC(=O)O] |
| DiAza- diglycerol | 432.51 | 431.6 | |
| of azelaic acid | | | |
| B- TriAza derivative- keto | 554.7 | 555.8 | 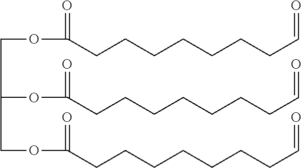 [SMILES: O=CCCCCCCCC(=O)OCC(COC(=O)CCCCCCCC=O)OC(=O)CCCCCCCC=O] |
| C TriAza derivative-mixed | 570.7 | 569.7 | 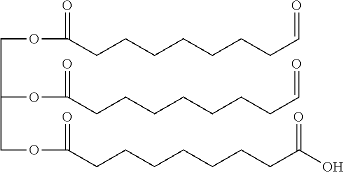 [SMILES: O=CCCCCCCCC(=O)OCC(COC(=O)CCCCCCCC(=O)O)OC(=O)CCCCCCCC=O] |
| D TriAza derivative mixed | 586.7 | 585.6 | 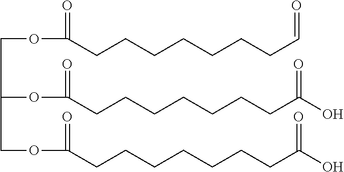 [SMILES: O=CCCCCCCCC(=O)OCC(COC(=O)CCCCCCCC(=O)O)OC(=O)CCCCCCCC(=O)O] |
| TriAza | 602.71 | 601.6 | 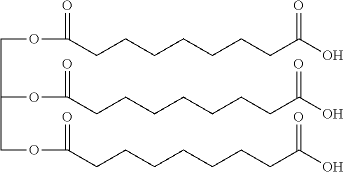 [SMILES: O=C(O)CCCCCCCC(=O)OCC(COC(=O)CCCCCCCC(=O)O)OC(=O)CCCCCCCC(=O)O] |
| F- di-olein derivative | 760.2 | 759 | 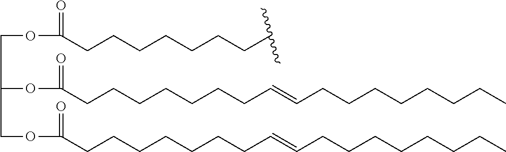 [SMILES: *CCCCCCCC(=O)OCC(COC(=O)CCCCCCC/C=C/CCCCCCCC)OC(=O)CCCCCCC/C=C/CCCCCCCC] |
| OOO- ozonide | 885.4 | 884.1 | Ozonide -OOO not oxidized |
| Tri-Aza precursor | | | |
| I | | 1081 | Dimer of Mm 601.6 |
| E- ozonide derivative Mono- Aza | 744.9 | 743.8-747 | 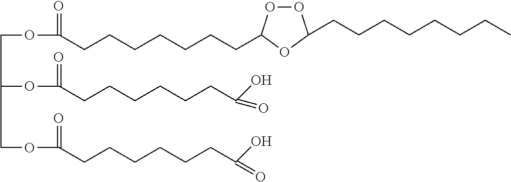 [SMILES: CCCCCCCCC1OOC(CCCCCCCC(=O)OCC(COC(=O)CCCCCCC(=O)O)OC(=O)CCCCCCC(=O)O)O1] |

### Example 14: Esterification Synthesis of TriAza as a Mixture of 2:1—Ratio of Azelaic Acid-Aza: Pelargonic Acid (PA)

A solution of DCC (725 mg, 3.5 mmol) and DMAP (430 mg, 3.5 mmol) in methylene chloride (10 ml) was added to a solution of glycerol (200 mg, 2.2 mmol), Aza (95%, 610 mg, 2.2 mmol) and PA (99%, 310 mg, 1.1 mmol) in methylene chloride (40 ml) at room temperature under nitrogen. As the reaction proceeded a precipitate dicyclohexylurea formed. After 5 h hexane (50 ml) was added to precipitate more dicyclohexylurea and the reaction was filtered and concentrated to dryness. Purification by flash chromatography (5% ethyl acetate/hexane) yielded the pure triglycerides as a colorless oil.

### Example 15: Esterification synthesis of TriAza as a Mixture of 1:2 Ratio Of—Pentadecanoic Acid-Azelaic MOCL Derivative of C15: Aza

A solution of DCC (725 mg, 3.5 mmol) and DMAP (430 mg, 3.5 mmol) in methylene chloride (10 ml) was added to a solution of glycerol (200 mg, 2.2 mmol), Aza (988, 305 mg, 1.1 mmol) and C15 (99%, 620 mg, 2.2 mmol) in methylene chloride (40 ml) at room temperature under nitrogen. As the reaction proceeded a precipitate dicyclohexylurea formed. After 5 h hexane (50 ml) was added to precipitate more dicyclohexylurea and the reaction was filtered and concentrated to dryness. Purification by flash chromatography (5% ethyl acetate/hexane) yielded the pure triglycerides as a colorless oil.

### Example 16: Esterification Synthesis of MOCL-TriAza as a Mixture 2:1 Ratio of Aza: RetA-Retinoic Acid as as Optional Ingredients of Olebiome Composition

A mixture of glycerol (200 mg, 2.2 mmol), Aza (988, 610 mg, 2.2 mmol), RetA (99%, 305 mmol, 1.1 mmol) and p-toluene sulfonic acid (20 mg) were heated at 140° C. for 5 h under a stream of nitrogen. The reaction was cooled and purified by flash chromatography (5% ethyl acetate/hexane) to yield the pure triglycerides as a colorless oil.

### Example 17: Esterification Synthesis of MOCL-TriAza Derivatives as a Mixture 1:2 Ratio of Aza: BrA-Brassylic C13 Dicarboxylic Acid Acid as Optional Ingredients of Olebiome Composition

Notably triglycerol of C13-brassylic acid classical oxidative cleavage of olefinic compounds with manganate/periodate system or metal-catalyzed processes. Oxidative cleavage of olefins with tungstic acid or co-oxidation with oxone was successful with 80% yield of pelargonic acid and C13 acid. A mixture of glycerol (200 mg, 2.2 mmol), Aza (98%, 305 mg, 1.1 mmol), BrA (99%, 620 mmol, 2.2 mmol) and p-toluene sulfonic acid (20 mg) were heated at 140° C. for 5 h under a stream of nitrogen. The reaction was cooled and purified by chromatography flash (5% ethyl acetate/hexane) to yield the pure triglycerides as a colorless oil. BrA obtained by oxidation of eruric acid-(˜50%) obtained from rapeseed oil by oxidative cleavage of raw rapeseed oil with tungistic acid H₂OWO4, resulted in high yield mix of PA and BrA at 80° C. during oxygen bubbling or oxygen 10 bar pressure for 12 hours.

### Example 18: Esterification Synthesis of TriAza Under QC-Quality Controlled Processing

Screening of end products of reaction after process was done in ozonolysis/oxidation (acidic pH) vs oxidation (acidic pH) only and ozonolysis/oxidation (at neutral or basic pH) reactions exemplified in Table 12 below, proving that TriAza can be obtained at acidic oxidation only, otherwise hydrolysis of TG results in FA release even if desaturation of bonds resulted in azelaic acid formation, glycerol bond was fully hydrolyzed. Therefore-PA is essential by-product and can serve as identity specification in QC parameters, but Aza is an indicator of resulted reaction not properly processed and can serve as impurity specification for QC release.

TABLE 12
Batch process conditions and end products

| Batch process/end | | | | |
|---|---|---|---|---|
| products | Oleic | azelaic | Pelargonic | TriAza |
| Ozonolysis/H2O2/Asc | − | − | + | + |
| Pelargonic/separation | − | − | + | + |
| Ozonolysis/O2- | − | − | + | + |
| propionic/succinic | | | | |
| acid, pH < 6-5,5 | | | | |
| Ozonolysis/oxidation, | + | + | + | − |
| pH 7-8 | | | | |

### Example 19: Testing Microbiome Balace Functionality of MOCL by Minimal Inhibitory Concentration to P. acnes by Microdilution Method

P. acnes after double passage on Chocolate plates (Novamed) were grown in anaerobic conditions at 37° C. for 1-2 days. The bacteria were Gram stained and the proper morphology of the bacteria was assured. Then, bacteria were resuspend in Thioglycolate medium

(HyLabs) at concentration of 0.02 O.D.600. Meanwhile, ELISA plates containing at the first raw undiluted tested material were prepared. The rest wells were filled with 100 μl of Thioglycolate medium and 11 serial ½ dilutions by transferring 100 ul of undiluted material was performed. Once the ELISA plate was ready, 100 ul of prepared bacterial culture was added. Bacteria alone and medium w/o bacteria were used as standard controls. The plates were incubated in anaerobic conditions at 37° C. for 2 days. Minimal inhibitory concentration was determined visually and spectrophotometrically, while minimal bactericidal concentration was determined after plating of the bacteria on solid medium (Chocolate plates). On the plates bacteria were incubated in anaerobic conditions at 37° C. for 2 days and the number of appeared colonies were counted manually.

### Results:

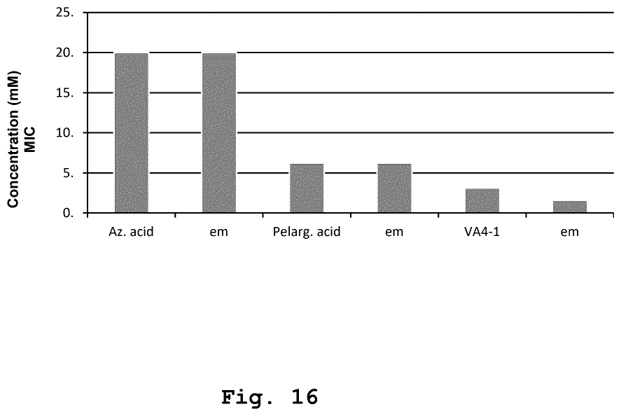

Screening antibacterial efficacy of C9-OCFA (azelaic-Az. Acid and Pelargonic-Pelarg. Acid) and MOCT-TriAza (batch VA4-1) with and without emulsifier, as shown in FIG. 16, proves that TriAza has the highest anti-growth bacteriostatic activity. MIC bacteriostatic activity of TriAza only, not C9 FA, is enhanced by use of surfactant or emulsifier, where 1 mM is sufficient to stop growth of the pathogen. Therefore, TriAza: formula will essential comprise “green” surfactant emulsifier in the disclosed composition

TABLE 13
Olebiome anaplerotic composition- based Formulas
for Biopotency and Efficacy Experiments

| Olebiome | | | |
|---|---|---|---|
| Composition | Formula 1 | Formula 2 | Formula 3 |
| Carrier oil | 70% Jojoba | 70% Olive | 70% Argan |
| comprising | oil/Sunflower | oil/almond | oil/sesame |
| triolein at | oil (50-50) | oil (60/30) | oil (80/20) |
| least 20% | | | |
| TriAZA/ | TriAza | Tg9 + Tg7 | TriAza/C9 |
| MOCFA/OCFA | 8%/Pelargonic | (50-50) 10%/isoC9 | diester- |
| | 10% | ester/diester | DiAza 10% |
| | | mix14% | |
| Ascorbyl | 10% | 5% | 10 |
| palmitate | | | |
| CoQ10 | 1% | 1% | — |
| Vitamin E | | | 10 |
| Acyl- | 1% | | |
| carnitine | | | |

### Example 20: Method of testing the “aged appearance” status of the skin

Method of subjective qualitative evaluation of skin whitening and smoothening at the test site and improvement in fine lines is evaluated subjectively by the investigator and/or by the subjects in accordance with the following scoring pattern:−3=marked deterioration;−2=moderate, visibly uneven deterioration;−1=slight deterioration; 0=no perceptible change or improvement; 1=slight change or improvement; 2=moderate change or improvement (whitening; perceptible and visible change, with less than 50% lightening of skin color); 3=marked or improvement remarkable change or improvement (whitening; very visible change with even and uniform skin whitening covering more than 80% of the contact area). Three study volunteers used TriAza for reduction of skin “aged appearance”-all three after month of use reported 2-3 scale of appearance improvement.

### Example 21: Method of measuring quantitative skin quality under

ongoing evaluation

Methods of quantitative skin quality under ongoing evaluation including transepidermal water loss (TEWL), is measured on the cheeks with a Tewameter® TM300 (Courage+Khazaka ElectronicGmbH), which measures water evaporation from the skin. For normal skin, under ambient conditions, TEWL oscillates between 4 and 10 g/h/m2. This water loss accounts for a total of about 500 ml per day but may increase up to 30 times higher when the epidermis is damaged. Therefore, TEWL correlates with skin barrier function and can be a measure of dysfunction. TEWL is regarded as an important parameter when measuring skin barrier integrity.

### Example 22: Method of measuring moisture level or hydration state of the skin of the face

The moisture level or hydration state of the skin of the face is measured with a Corneometer® (Courage+Khazaka Electronic GmbH, Cologne, Germany). As with the Mexameter®, the test site is the cheekbone area. The Corneometer® uses capacitance to measure the moisture content of the stratum corneum.

### Example 23: Method of measuring aging appearance using the grade of Crow's feet wrinkles

Measurement of aging appearance using the grade of Crow's feet wrinkles (GCFW) is a standardized method by 1-6 point scale skin smoothening. GCFW, i.e. “crow's feet” at the outer corner of the eye can be determined by clinical scoring of the Crow's feet wrinkles. Alternatively, digital imaging systems to test site for wrinkle reduction and SkinSys software (Sometech Inc, Seoul, South Korea) and a Coccam digital camera (Beauty Korea World Co Ltd, Seoul, South Korea) are used. SkinSys is professional skin analysis and treatment software that enables the user to perform a well-organized scientific evaluation of cosmetic products. The skin analysis and measuring functions include: 1) 3D analysis of skin curvature (wrinkle reduction); and 2) analysis of keratin content by using a precise edge-detection algorithm. To test effect of wrinkles subdermal injection of TriAza Formulas 1-3 (Table 13) are coadminstrated with botox and hyaluronic acid and elasin for evauation.

### Example 24: Method of measuring skin elasticity

Skin elasticity is quantified with a Triplesense TR-3 sensor scan device (Schott Moritex Corporation, Saitama, Japan). To test elasticity, samples of Olebiome C999 formula OCFA/MOCT Formula 2 was preconditoning for topical administration of low molecular weight hyaluronic acid for evaluation.

### Example 25: Method of measuring colorimetric skin-whitening efficacy

Colorimetric skin-whitening efficacy are quantified by means of the melanin index using a Mexameter® MX18 (Courage+Khazaka Electronic GmbH, Cologne, Germany). Whitening efficacy is tested in the cheekbone area. The melanin index recorded is the average of three readings. The measurement is based on the absorption principle. The probe emits light of three defined wavelengths and a receiver measures the light reflected by the skin and thus the light absorbed. The melanin is measured by using two of the three wavelengths, which are chosen to correspond to the different rates of light absorption by the melanin pigments. Formula 1 (Table 13) of Olebiome C999 with Asc acid, CoQ10 and Acyl carnitine are provided below.

### Example 26: Wound healing compositions

The wound healing compositions of Table 13 is formulated for cell culture experiments and may be utilized in topical products, ingestible products, and tissue culture medium to protect mammalian cells and increase the resuscitation rate of injured mammalian cells.

### Example 27. Measuring reduced levels of hydrogen peroxide

Mammalian epidermal keratinocytes can be employed to examine the ability of various antioxidants to reduce levels of hydrogen peroxide in these cells. Hydrogen peroxide levels are measured after the cells are exposed to ultraviolet light in the wavelength range from 290 to 320 or nm (UV-B) to the inflammatory compound 12-O-tetradecanoyl-phorbol-13-acetate (TPA). TriAza Formula 1 and 3 at concentration 0,5-1% of culture media dish and can be tested at various concentrations to determine the effect of concentrations of this antioxidant on the hydrogen peroxide production by epidermal cells. Mammalian epidermal keratinocytes are isolated by trypsinization of epithelial sheets and grown in modified basal MCDB 153 medium supplemented with epidermal growth factor, 10% fetal calf serum and hydrocortisone. Cells are maintained in a humidified incubator with 5% carbon dioxide at 37° C. Keratinocytes are seeded in 60 mm culture dishes at a cell density of 3×10 cells per dish and the cultures are exposed to 1 M.E.D. dose of ultraviolet-B light (100 mj/cm) or treated with 100 ng/ml of TPA. TriAza cell culture. Formulas 1-3 (see Table 13) are dissolved in 2% DMSO and 2% surfactant (PVP). The appropriate concentration of test solution or combination of test solutions are added to the cells immediately prior to exposure of the cells to ultraviolet light-B or TPA [100 ng/ml]. Stock solutions are prepared so that the vehicle did not constitute more than 1% of the total volume of the culture media.

Intracellular hydrogen peroxide production by mammalian epidermal keratinocytes cab be measured using dichlorofluorescein diacetate (DCFH-DA, Sigma). DCFH-DA is a non-polar non-fluorescent compound that readily diffuses into cells where it is hydrolyzed to the polar non-fluorescent derivative DCFH which then becomes trapped within the cells.

In the presence of intracellular hydrogen peroxide, DCFH is oxidized to the highly fluorescent compound DCF. Hence, cellular fluorescence intensity is directly proportional to the level of intracellular hydrogen peroxide produced. Cellular fluorescence intensity can be monitored by fluorimetry and by flow cytometry. Mammalian epidermal keratinocytes (1×10⁶ per dish) are incubated at 37° C. with 5 uM of DCFH-DA. Production of hydrogen peroxide is measured using a Coulter Profile analytical flow cytometer. Each analysis is repeated three times and the quantitation of fluorescence is expressed in terms of femtomoles (fmol, 10 moles) of DCF oxidized per cell, which is a direct measure of the intracellular hydrogen peroxide produced. All comparisons are assessed against the controls, which produced 250 hydrogen peroxide-H—O-fmol/cell. The positive numbers represent H₂O: production in excess of the control and the negative numbers represent H₂O: production below the control.

C999 composition of Formula 1-Table 13 resulted in 2,5-5 folds reduction of HO: production in comparison to control, for 0,5-1% concentration of the TriAza comprising formulas (Formula 3-Table 13) only at concentration 0.25%-0,5% resulted in same range of activity as C999 Olebiome (Formula 2-Table 13).

### Example 28. Alternative cell culture experiment of Olebiome

TriAza −Measuring reduced levels of hydrogen peroxide

Additional experiments of protective epidermal cell barrier effect can be performed by measurement of TBAR activity under divalent iron induced oxidative stress resulted in lipid peroxidation (LPO) ex vivo. Human keratinocytes tissue (Mattek, EpiOral) culture are preincubated with Formula 1, Formula 2 and Formula 3 (Table 13) during 30 min at 37° C. Afterwards, freshly prepared FeSO4.7HO are added to induce lipid peroxidation and slices are incubated for another 15 min at 37° C. with gentle shaking under permanent oxygen aeration. At the end of the incubation period, medium is separated from the tissue by centrifugation at 3500×g for 5 min and 0.5 ml of medium is taken for thiobarbituric acid-TBA reactive substance (thiobarbituric acid reactive substance-TBARS) content determination. Not treated control under LPO stress is evaluated as 100% of oxidative stress damage. The most prominent inhibition of TBARS production (about 60%) is observed after Formula 3 (reasonable impact of vitamin E), Formula 1 and 2 results in 25% and 40% TBARS reduction respectively.

### Example 29. Examination of Olebiome compositions TriAza −Formula 1 and C999 MOCT/OCFA-Formula 2 on functionality assay for wound gap closure-scratch assay

In vitro wound healing assay can be performed by measuring percent gap open for a wound created on fully confluent culture of primary gingival keratinocytes (ATCC@ PCS-200-014™) treated with Formula 1 and 2. 1% of total culture media dish is added from each Formula 1, 2 and 3 dissolved in DMSO and prepared as described above. Images are acquired at 8; 12; 16 and 24 hrs., post administration under a phase contrast microscope. Media only on gap closure is utilized as control. For the control group the wound gap closed at <50% at 24 hrs. For Formula 1 and 3 treatments >80% closure is seen in 12 hours, 100% closure is seen at 16 hrs. For Formula 2 and 3>80% closure is seen at 24 hrs. All Formulas have wound closure potential for the protection of epidermal mammalian cells barrier.

### Example 30: Use of the TriAza composition for food

To test dietary/functional food effect of TriAza composition on healthy and patients' metabolome pattern, the following metabolomic tests are performed:

1.

Analysis of quantitative urinary organics acids by GC-MS (mmol/mol creatinine), including: 3-OH-propionate, heptanoate, octanoate, methylmalonate (MMA), 3-OH-pentanoate (BHP), 3-keto-pentanoate (BKP), pimelate, methylcitrate, pyruvate, acetoacetate (AcAc), lactate, 3-OH-butyrate (BHB), 2-oxoglutarate, succinate, fumarate, malate, aconitate, isocitrate, citrate adipate, suberate, and sebacate.

2. Blood chemistries include tests for:
glucose, potassium, CO2, anion gap, BUN, creatinine, albumin, AST (SGOT), ALT (SGPT), ammonia, GGT, creatine kinase (CPK), cholesterol, triglycerides, HDL, and LDL. Blood levels of 3-OH-butyrate (BHB), acetoacetate (AcAc), 3-OH-pentanoate (BHP), 3-ketopentanoate (BKP), free carnitine.

Subjects are instructed to consume 10% (1^{st} group: two snacks per day), 20% (2^{nd} group: four snacks per day) of their estimated total energy needs from the study oil during the study.

Based on FDA approved triheptanoin (C7 MOCT) safe dose-˜ 35% of general intake1^{st} will group receive the composition standardized to 50 g TriAza+ ‘PA per day, and 2^{nd} group, crossover design 100 g per day, but not higher that 150 g day (divided in two or four meals (snacks) per day.

Suppliers for synthetic TriAza are oleic excipients producers such as IOI Olea GmBH, Stepan Inc., Tx-USA, or SASOL, GmbH, Mead-Johnson Nutritionals, or other similar lipid oil synthesis producers or Dr. Reddy synthetic oxidation expert group.

### Regimen

Recommended adjunctive low fat-low carbohydrate diet is maintained along with two or four “snacks” in Rainbow kits. Rainbow packages used as spread & dip sauces, or as dressing oil for salads, vegetables or low carb low fat low sugar recommended diet. With the gradual decrease in caloric requirement with age, the amount of ˜ 1.0 gm/kg/day should be sufficient for MOCT intake.

Microbiome and biomarkers of hallmarks of aging (https://en.wikipedia.org/wiki/Hallmarks_of_aging) related tests.

The effect of microbiome-enriched composition as a food or nutritional supplement can be clinically tested metabolomics, on genomic stability and telomer protection on suitable models. In vitro tests related to healthy microbiome protects telomeres and genomic integrity against cellular stress, commensal microbiome species prevalence has been shown to be associated with longer telomers in some animals. In vitro and ex vivo tests of Antiaging effect can be tested by biomarkers characterization through autophagy and senolytic markers, SIRT enzyme assay, COX assay (thromboxane and other prostaglandins release) and IDO assay.

### Example 31: Preparation of hydrophilic compartment of biomimetic longevity functional food anaplerotic amino acids

To prepare hydrophilic compartment of biomimetic longevity functional food anaplerotic amino acids (AAA) comprising protein, hydrolysate was mixed during heating with trehalose/xylose syrup and hydrophilic longevity inducing active component. Prior to mixing, the special longevity inducing active biomimetic sugar trehalose/xylitol mixture is heated at minimum 30° C. until boiling to form syrup in water at ratio 1:1-1:2 sugars in water. Then, chocolate carrier and surfactant are mixed. Next, the mixture of the chocolate-green surfactant formed as carrier for biomimetic longevity inducing active composition and then, hydrophilic mixture compartment as above is added while stirring to emulsify. The final biomimetic chocolate spread is prepared by adding and stirring TriAza to the emulsion as described above. Optional probiotics can be added to complete longevity inducing active biomimetic functional or medical food composition.

### Example 32: Methods for producing a spread-dip type chocolate composition

The following procedures relates to various methods for producing a spread-dip type chocolate composition, and to a method for producing a functional/medical food chocolate spread comprising a TriAza, longevity metabolic mimetic, and AAE, including autophagy inducers (wheat germ-spermidine enriched extract and trehalose) in chocolate spread-dip composition:

1. Phase-1: Dilute hot extracted wheat germ extract/oil (comprising at least 1-2% of spermidine), hydrolyzed protein source, e. g. (specified gelatine with amino-acid content enriched with glycine 1 with selected special sugars, i.e. trehalose/xylose (at ratio=1:4-1:10) syrup mix during heating, cool and during cooling homogenize with hydrophilic AAE (i.e. carnitine, ascorbic acid, succinic acid, citric acid, lactic acid, choline, adenosine, indoles, SCFA—e.g. butyric acid) and derivatives of thereof.

2. Phase-2: Add 1 part of high-quality melted chocolate for 2 parts of hydrophilic Phase 1 water and homogenize during heating, optionally add natural amphiphilic plasticizer (C9 esters) and/or, solvent (isopropyl myristate/isopropyl oleate), surfactant (preferably GML-glycerol monolaurate).

3. Phase-3: Add lipid/fat (vegetable oil, butter, PC/PS, cholesterol, MCT) composition fortified with OCFA/MOCT-C999 (5-10%) and/or precursors and derivatives of thereof and lipid-soluble vitamins and ARE not >1-2% each (e.g. Vitamin E and Vitamins group B, e.g. niacinamide, caffeine, polyphenols, terpenes, e.g. limonene, coQ10, phytosterols, flavors vanillin, not more than 10% total, bile acids, etc.), and homogenize under air free environment-closed mixer, nitrogen stream at 35° C. for two hours.

4. Optionally, Phase 1 is added as a dry powder mixed into oil phase directly with chocolate premixed with oil phase and surfactants and homogenized during warming.

5. When total homogenate is cold-preferably add probiotic such as bifidum, acidophilus, lactobacillus, 124 actococcus, selected protective longevity-related microbiome, pre-packed and maintained under sterile/aseptic conditions before and during mixture to prevent contamination.

Surprisingly, the prevalence of oily phase (since the chocolate spread is a water in oil emulsion) mix of C9 in oil with vitamins, surfactants, xylitol, and acids all serve as natural preservatives and therefore control probiotic stability and prevent contaminations. In addition, even MCT, and derivatives are screened as useful natural preservatives of the composition, GML-glycerol monolaurate is selected as an adjunctive multifunctional natural preservative to MOCL based composition. S. aureus spiked contamination microbiologically screened and best results for antimicrobial activity represented by natural preservative selected-glycerol monolaurate-GML emulsifier:
MIC=17, 5 mmol with C9-triisononanoin+GML; MIC=23 mmol with tripelargonin; MIC=5 mmol with diethylester of azelaic acid+GML; MIC=2, 5 mmol of TriAza+GML).

### Example 33: Alternative methods for producing a spread-dip type chocolate composition

The following describes another optional method of preparation of chocolate spread longevity food:

1. Prepare Phase 1-Hydrophilic compartment: consisting of carb-hydrolyzed protein mixture (AA mix preferably based on anaplerotic amino acids enriched in hydrolyzed collagen/gelatine mix, AA metabolite mimetics), hydrophilic AAE and probiotic mix: heating at temperature 50-70° C. the mixed probiotic and hydrophilic AAE and Trehalose-xylose to form a mixed amphiphilic syrup, the preferable ratio of xylose/trehalose is 1:4-1:5; preferable ratio of carb/sugars to protein 3:1-4:1-5:1. Essential AAEs are hydrophilic creatine (ratio creatine/protein=1:5-1:10), essential longevity biomimetic AAE is spermidine 1% (preferably wheat germ extract specified with 10-20% spermidine), adenosine (200-500 mg/dose), optionally hydrophilic vitamins and longevity actives (AscA, choline, succinic acid, indoles).

2. Prepare 2-Emulsifying compartment: Mixing the chocolate with selected natural sourced green surfactant (GML, glucoside based, C9 ester-based plasticizer), optionally green solvent (such as C9 alcohol, isopropyl myristate, isopropyl oleate, isopropyl and palmitate.

3. Add the Phase 1-hydrophillic compartment (AA/hydrolyzed protein and carb) of mixed syrup to the Phase 2-emulsifying mixture of the chocolate and then form a stirred water in oil emulsion.

4. Add C9 fortified lipid Phase 3-C9 lipid/oil/fat hydrophobic compartment with hydrophobic AAE essentially comprising vitamin E (e.g. optionally comprising coQ10, caffeine, vit B-niacinamide, phyto-protectants, such as phenols, flavanols, and terpenes to the emulsion, and stirring the emulsion until uniform homogenate texture at medium heat 30-40° C. optionally in closed homogenizer and/or under nitrogen/argon gas to prevent oxidation).

### Example 34: Use of TriAza as a functional balance-biotic in milk

Culture medium (consist with dry milk/non-fat) was prepared based on TriAza (25%), selected novel prebiotic mixture (crude fiber, inulin, xylitol, vanillin, date honey) and selected mixture of probiotics including: Lactobacillus acidophilus,

Bifidobaterium lactis, Lactobacillus rhaminosus, and Lactobacillus bulgaricus. Culture was kept in simple fermenter with low oxygen at 25° C. at dark dry place. No yeast, fungal or external bacterial contamination was determined.

### Example 35: SPF boosting effect of Olebiome Formula 8

The objective of SPF study was to determine the SPF booster capacity of Formula 8 was enriched with apricot oil and comprise following ingredients as per INCI names: Olea Europaea Fruit Oil, Ethylhexyl Pelargonate, Isononyl Isononanoate, Triheptanoin, Prunus Armeniaca Kernel Oil, Tripelargonin, Ascorbyl Palmitate, Ubiquinone. This formula was tested as per in vitro measurement for sunscreens as per guidance in ISO 24 443. Measurements were made with a Labspeère UV 2000S, compared to a reference standard with a determined SPF (Monaderm P5). VR08019-1.08 oil was incorporated at 10% into a sun cream SPF30, reference MONADERM P5. An in vitro SPF measurement was then performed on this mixture. Comparatively, a mixture of the reference SPF30 sunscreen containing 10% Caprylic/Capryc

Triglycerides was also measured. The test SPF levels of reference MONADERM P5+10% caprylic/capric triglyceride was 34, and for Olebiome tested batch, the SPF level was 43.

Surprisingly, the results showed that Olebiome composition can be used as an effective UV filter compound to boost SPF at 25%-40%, therefore reducing concentration of conventional sunscreen filter compounds.

Table 14 summarizes the study parameters:

TABLE 14
SPF measurements

| Test | SPF | UVA | Critical wavelength (nm) |
|---|---|---|---|
| Reference MONADERM P5 | 30.06 | 13.40 | 378.00 |
| Reference MONADERM P5 + | 34.05 | 14.93 | 377.20 |
| 10% Caprylic/Capric | | | |
| Triglyceride | | | |
| Reference MONADERM P5 + | 42.92 | 18.35 | 377 |
| 10% Olebiome Formula 8 | | | |

### Example 36. In-vitro assessment of irritating potential using the neutral red release assay

The irritation potential was assessed in-vitro by directly applying the Olebiome Basic multipurpose Formula 7 comprising following Ingredients: Olea Europaea Fruit Oil, Ethylhexyl

### Pelargonate, Isononyl Isononanoate, Triheptanoin, Prunus

Armeniaca Kernel Oil, Tripelargonin, Ascorbyl Palmitate,

Ubiquinone was assessed in-vitro by directly applying to rabbit corneal fibroblast monolayers and evaluating its effects using the neutral red release assay.

To assess its cytotoxicity Olebiome Basic multipurpose Formula 7 was diluted in paraffin oil and put in contact with SIRC fibroblasts preloaded with Neutral Red vital dye. The cells were exposed to concentrations of test product for a fixed period. The percentage of cell death was calculated for each tested concentration. The cytotoxicity of the test item was determined by the evaluation of the IC50 (concentration inducing 50% cell death) value. No significant cytotoxic effect (cell death <10%) was observed in the range of all tested concentrations. The IC50 was estimated to be higher than 50%. According to the quotation scale, the cytotoxicity of the test item is considered negligible. Therefore, Olebiome was proven as a non-irritating SPF booster. Since some SPF agents are known as irritating ingredients like avobenzone, oxybenzone and others, Olebiome compositions were tested for irritating test in dermatological clinical study as described below.

### Example 37. Clinical in-vivo test of Olebiome Multipurpose oil for dry skin

Dermatological test to assess the presence of an allergic reaction or contact eczema. In vivo skin irritation method-semi-open test conducted on 10 subjects with a history of allergies. The purpose of the clinical GCP study was to assess irritating properties (skin tolerance) of the product on healthy adult skin, with applied patch test. The skin at the application spot (arms or interscapular area) was healthy, without lesions. The preparation in appropriate concentration is applied onto filter paper discs of 12 mm diameter, manufactured by

SmartPractice® and then fixed to the arm or interscapular area with the use of a sticking patch. At the same time, to guarantee objective results of the study and to exclude possible reading errors connected with dermal irritations, two control samples (control sample called “blind” and control sample containing water) are used. The dermatologist removes the patch 48 hours after the application and examines the skin reaction 30 minutes after the removal. 72 hours after the application, the dermatologist examines the skin again for a reaction. If irritations appear or persist 72 hours after the application, an additional examination takes place after 96 hours. While determining the skin reaction, the dermatologist assesses the irritating and sensitizing effects of the tested product. The study allows to conclude that product Multipurpose oil for Dry skin Olebiome Formula 8 Armeniaca Kernel Oil, Triheptanoin, Ethylhexyl Pelargonate, Tripelargonin, Ascorbyl Palmitate, Ubiquinone) used by volunteers, who didn't report documented oversensitivity or a history of adverse reactions to individual ingredients of the tested product, is well tolerated by the skin. In the tested group of volunteers there were no irritations or allergic reactions. The product meets the requirements of compatibility test with the skin (Skin Compatibility Test) and can be classified as non-irritating.

### Example 38. Cell viability Upon Peroxidation-ROS Stress

(hydrogen peroxide treatment) and Mitochondria Functionality: ATP and ROS Assay.

To evaluate the effects of Olebiome on mitochondria, ATP levels and overall intracellular ROS (DCFDA) in control or Olebiome-treated cells. Fibroblast cell lines were cultured in MEM supplemented with 10% FBS and 2 mM L-glutamine (all reagents-Life Sciences-Invitrogen brands) at 370C with 5% CO₂.. Olebiome formula at concentration 10% per cell culture well volume as dissolved in DMSO and added to the growth medium. To induce oxidative stress, Olebiome-treated skin fibroblast and control cells were incubated with PBS containing HO: at 1 mM for 60 minutes. The cells were then washed three times using PBS and subjected to a cellular stress assay. Intracellular ATP content was measured by using luminescence ATP detection system (ATPlite, PerkinElmer). After the cells had been harvested with 0.05% trypsin, lysed with the lysis buffer for five minutes, the substrate solution was added and mixed for another five minutes to conduct the reaction for light generation. After dark adaptation, the luminescence intensity of each well was acquired on SpectraMax M5 Microplate Reader. Improvements in ATP production (15-20%) and reductions in ROS levels (30-50%) (DCFDA, measured by FACS analysis) were found in Olebiome-treated cells compared to control. In addition, it was shown that hydrogen peroxide caused a significant reduction in cell viability (MTT assay), and both low (10%) and high (25%) concentrations of Olebiome improved cell viability back to control level.

### Example 39. Rapid effect of Olebiome on face skin moisture loss

within four hours post administration on aged volunteers including dry and sensitive skin subjects.

Sun exposure and radiation stress may lead to fast loss of skin hydration. The immediate effect of Olebiome composition was tested after 4 hours post application by clinical TEWL test. The aim of the study was to confirm the action prevent water loss by epidermis-transepidermal water loss within 4 hours. The test was conducted with a special measuring device of the Courage+Khazaka Company-Tewameter@ TM 300. An instrumental study was performed on the face skin. One half of the face was the test zone, the other was the control zone. Directly before the measurements the subject's skin was not treated by other products. Before the product application the measurements of TEWL were done in each zone in all tested and controlled places. Next the measurements of TEWL were performed before (to) and after 4 hours (t4 h) from the product application. Each final result is the arithmetic mean of individual values obtained during a measurement lasting a minimum of 20 seconds. The study was carried out in an air-conditioned room with the temp. of 20+2° C. and relative humidity 50+10%.

Subjects’ selection: study designed to prove applicability of Olebiome SPF antiaging booster >25% of subjects were >60 years old, >50% of subjects were >50 years old, all subjects were >35 years old. Diverse subjects were recruited males and females, with dry, combined and sensitive skin types to prove multifunctionality and universality of SPF booster. Since aged population and effect of age related and external stresses such as solar, radiation and chemotherapy are usually relevant for skin over 50% of subjects recruited were dry-skin type. Results: as per clinical dermatology guidance product's efficacy is confirmed in the case of the positive results obtained in more than 50% of subjects—it was proven that Olebiome composition reinforces hydro-lipid barrier within 4 hours after application in 100% of aged subjects, see Table 15 below:

TABLE 15
Tewameter ® TM 300.

| Subject's no. >age 50 | t4h |
|---|---|
| 1. >50 | −0, 4 |
| 2. >50 | −1, 0 |
| 3. >50 | −1, 1 |
| 4. >50 | −0, 2 |
| 5. >50 | −0, 6 |
| 6. | −1, 3 |
| 7. | −1, 5 |
| 8. | −1, 1 |
| 9. | −1, 2 |
| 10. | −0, 2 |
| 11. >50 | −1, 1 |
| 12. >50 | −0, 6 |
| 13. >50 | −1, 0 |
| 14. >50 | −0, 8 |
| 15. >50 | −1, 2 |
| 16. >50 | −1, 4 |
| 17. >50 | −1, 4 |
| 18. >50 | −0, 6 |
| 19. >50 | −0, 8 |
| 20. >50 | −0, 9 |
| 21. >50 | −1, 1 |
| Mean | −0, 9 |
| Min | −1, 5 |
| Max | −0, 2 |
| SD | 0, 4 |
| Median | −1, 0 |
| delta % | −10% |
| % subjects with positive effect | 100% |

The results of TEWL (transepidermal water loss) measurements after 4 hours (t4 h) from product application in comparison to the control zone in g/h/m2. Values of TEWL (transepidermal water loss) in g/h/m2 in comparison to the control zone.

### Example 40. Development and quality characteristics of food

### Olebiome Oleogels.

Oleogels that were developed are composed of waxes in edible oils in order to replace explored for various food applications, including solid chocolate and semi-solid spreadable (“spread and dip™”) products. Rice bran wax, candelilla wax, carnauba wax, and beeswax, have been screened as being safe (GRAS) by the FDA. Favorable oxidative status and storage stability between screened formulations based on Olebiome composition composed of: 1. Olive oil ˜30% of total formula, was selected due to high Triolein levels (about 80%) and polyunsaturated fatty acids as well as its natural antioxidants such as tocopherols and polyphenols, which contribute to its nutritional value and oxidative stability. However, risk of initiation and propagation stage of lipid peroxidation reaction during storage is lower in medical, pharmaceutical grade olive oil see Table 16-Peroxidation Value (PV)<2. Final concentration of Triolein-enriched pure (QC tested) olive oil.

2. MOCL blend which includes noananoic and heptanoic glycerol and nonglycerol esters blend, and mixture of Aza, TriAza and DiAza, at concentration ˜20%. Aza derivatives were prepared by oxidation of ozonated olive oil and esterification respectively.

3.

Hydrophilic stabilizers (at 20% of total Olebiome formula). Screening revealed that non-polar antioxidants such as alpha tocopherol are more active in oil-hydrophilic compartment of oleogel, and polar Trolox (water soluble) and ascorbic acid was more active in oil compartment. Hydrophilic stabilizers-antioxidants were selected, namely Trolox and ascorbic acid and blended with hydrophilic biopolymers gelating stabilizers were selected (xylitol, alginate and gelatin mixture); 4.

Lipophilic oleogel stabilizers ˜30%.

Glycerol monolaurate-GML was selected as oleogel-inducer with emulsifying dispensing stabilizer with desirable physicochemical characteristics solid at RT and melted in mouth-melting point 50. Surprisingly, Olebiome oleogel best protection from oxidation during the process and storage was achieved with following stabilizers selected: GLM-glycerol monolaurate emulsifier (melting point 50-56° C.). By screening Olebiome formulations with formulated lipid stabilizers, the PV of the oleogels were higher than in raw materials but lower than those of bulk oil during storage without stabilizers. Especially low results were shown with formulas with GML and mix of waxes with melting point about 80° C.: rice bran wax (INCI Oryza sativa cera): melting point 79-85° C. and shellac wax with melting point 60-80° C. Lipid PV during TriAza oxidation processing reduced 50% with waxes, and twice when GML added. Final total concentration of lipophilic oleogel stabilizers-gelators, as selected-cacao butter, shellac wax and rice wax. Less of 5% comprise mixture of flavoring and lipophilic antiaging/longevity boosting ingredients, and/or probiotics to be added as per target product profile to waxes with GML blend during homogenizing and processing.

TABLE 16
PV parameters for QC specifications of Olebiome formulations

| | |
|---|---|
| QC test | PV specification, mEq/kg |

RM QC

| | |
|---|---|
| Ozonated olive oil | >1000 |
| MOCL esters blend without TriAza | <1 |
| TriAza enriched rm (ozonated, esterified, | 30-50 |
| epoxidated/oxidated, etc.) | |
| Olive oil (nonmedical grade) during storage | >3 |
| Olive oil pure medical grade during storage | <2 |

In process QC

| | |
|---|---|
| TriAza 0,5-50% MOCL blend | 10-30 |
| Olebiome processed formulation | >5 <30 |
| Final Olebiome product PV QC specification | <30 |

QC parameters selection shown in Table 16: ISO/TC 34/SC 11 has specified a method for the iodometric determination of the PV of animal and vegetable fats and oils with a visual endpoint detection. PV is a measure of the amount of oxygen chemically bound to oil or fat as peroxides, particularly hydroperoxides. The PV is usually expressed in milliequivalents (mEq) of active oxygen per kilogram of oil. Multiplication of the PV (mEq of active oxygen per kilogram) by the equivalent mass of oxygen (equalling 8) gives the milligrams of active oxygen per kilogram of oil. The method is applicable to all animal and vegetable fats and oils, fatty acids, and their mixtures with PV from 0 mEq/kg to 30 mEq/kg (milliequivalents) of active oxygen per kilogram. In Olebiome formulation relatively high PV was observed during the process but as hydroperoxides, which are the primary oxidation products, further oxidize to aldehydes and ketones, which are the secondary oxidation products, measured as TBARS. Based on formulation experiments and stabilizers selection, we selected QC specification for “in process-QC” and “final product QC” of Olebiome formulation that was specified as PV=5-30 mEq/kg, while for raw materials (RM) “incoming rm QC” PV<5 was selected as specification to reduce LPO reaction risk during storage and keep LPO in partial oxidation stage as specified for Olebiome composition. PV in the range of 1-5 mEq/kg indicates a low level of initial oxidation development, is a criteria selected for raw material (carrier oil and MOCL non-TriAza source RM, i.e., pure olive oil selected as RM with QC PV<3 mEq/kg, MOCL of non TriAza source PV<2 mEq/kg. For TriAza RM PV exceeding 10 mEq/kg signify a high level of process of oxidation to final TriAza ingredient with traces of oxidation products impurities, wherein intermediate raw material source of oxidation precursor products (e.g. PV for ozonated oil may exceed 30 mEq/kg, even reach 1500 mEq/kg for olive oil post ozonation). PV values 5-10 mEq/kg are considered to indicate a moderate level of oxidation for “in process QC” is acceptable and expected in Olebiome formulation since stabilizers included during heating process of the formulation to moderate TriAza partial oxidation precursors with lipophilic antioxidants, e.g.

ascorbyl palmitate-AP. PV screening by commercial kit for Olebiome Formula 7 and Olebiome Formula 8 after a year of shelf-life at RT is >5 mEq/kg, no change of color and no nonanal rancid scent, no change in turbidity, full transparency appeared at visual and sensorial testing. To reach PV balance between desired TriAza precursor and source impurities and inhibition of

LPO chain reaction during process and storage, anhydrous oil formulations of Olebiome are preferably selected, or water free formulations Oleogels with optional hydrophilic stabilizers, most preferably “green” stabilizers selected.

Oleogels can be prepared by stabilization of high oil/lipid content (70-90%) emulsion, or/and colloid suspension with “green” stabilizer such as surfactant, biopolymer, or a mixture of surfactant and biopolymer, as above at 0.5-10% concentration. Utilized here “green” stabilizers are natural, sustainable biopolymers such as gelatin, xanthan gum, glycolipids, lipoproteins, amphiphilic moieties, green emulsifiers, surfactants and lipopeptides can be applied. Representative, non-limiting examples that can be used for natural/“green” stabilizers are: GML, MONTANOV™ 68 MB, an O/W “green” emulsifier (Seppic); n-palmitoyl-Lys-Thr-Thr-Lys-Ser as pentapeptide marketed by Sederma under reference Matrixyl®, or n-palmitoyl-Gly-Val-Val-Ala-Pro-Gly as lipohexapeptide, which is marketed by Sederma under reference Dermaxyl®.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

It will be understood that when an element is referred to as being “on,” “attached” to, “operatively coupled” to, “operatively linked” to, “operatively engaged” with, “connected” to, “coupled” with, “contacting,” etc., another element, it can be directly on, attached to, connected to, operatively coupled to, operatively engaged with, coupled with and/or contacting the other element or intervening elements can also be present. In contrast, when an element is referred to as being “directly contacting” another element, there are no intervening elements present.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Throughout this application, various embodiments of this invention may be presented in a range format.

It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Whenever the term “about” is used, it is meant to refer to a measurable value such as an amount, a temporal duration, and the like, and is meant to encompass variations of +20%, +10%, +5%, +1%, or +0.1% from the specified value, as such variations are appropriate to perform the disclosed methods.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, and medical arts.

All publications, patent applications, patents, and other references mentioned in the disclosures of these publications in their entireties are hereby incorporated by reference into this application in order to more fully describe the state of the art to which this invention pertains. In case of conflict, the patent specification, including definitions, will prevail. In addition, the materials, methods, and examples are illustrative only and not intended to be limiting. Throughout this application various publications, published patent applications and published patents are referenced.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description. While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention. Various embodiments have been presented. Each of these embodiments may of course include features from other embodiments presented, and embodiments not specifically described may include various features described herein.

1. An anaplerotic Olebiome composition comprising
a) a blend of anaplerotic MOCL esters;
b) a Triolein-enriched plant carrier oil, and
c) at least one lipophilic antioxidant and/or antiaging ingredient.

2. The anaplerotic Olebiome composition of claim 1, wherein the blend of MOCL esters is at least one of:
a. enriched with glycerol and non-glycerol esters of nonanoic monocarboxylic and dicarboxylic acids, or any precursors and derivatives thereof;
b. comprises a plurality of nonanoic fatty acid derivatives and/or precursors, wherein the and/or precursors derivatives are selected from heptanoic acid, dicarboxylic nonanoic acid, and/or at least partially oxidized Triolein derivative;
c. comprises a plurality of nonanoic fatty acid ester derivatives, wherein said derivatives comprise a partially oxidized dicarboxylic derivative of Triolein, and wherein said partially oxidized derivative of Triolein is selected from azelaic acid, ester of azelaic acid, diester of azelaic acid, or any combination thereof; or,
d. any combination thereof.

3. The anaplerotic Olebiome composition of claim 1, wherein the anaplerotic Olebiome composition is anhydrous, and wherein the total MOCL esters concentration in the anaplerotic Olebiome composition is between 25% to 70%.

4. The anaplerotic Olebiome composition of claim 1, wherein the Triolein-enriched plant carrier oil is selected from the group consisting of olive oil, apricot oil, argan oil, almond oil, sunflower oil, VHOSF, cannabis oil, CBD oil, soybean oil, avocado oil, and rice oil, sesame oil, and mixture of thereof, and, optionally, wherein Triolein-enriched plant carrier oil is edible plant carrier oil.

5. The anaplerotic Olebiome composition of claim 1, wherein the concentration of the Triolein enriched plant carrier oil is between 25% to 85%.

6. The anaplerotic Olebiome composition of claim 1, wherein at least one lipophilic antioxidant ingredient is lipophilic anti-aging agent.

7. The anaplerotic Olebiome composition of claim 6, wherein lipophilic anti-aging agent is selected from lipid soluble derivative of ascorbic acid, ascorbyl palmitate, mitochondrial/longevity-boosting ingredient, or any combination thereof.

8. The anaplerotic Olebiome composition of claim 7, comprising two lipophilic anti-aging agents selected from ascorbyl palmitate and coQ10.

9. The anaplerotic Olebiome composition of claim 1, wherein Triolein-enriched plant carrier oil comprises at least 30% by weight of Triolein.

10. The anaplerotic Olebiome composition of claim 1, comprising less than 30% of hydrophilic ingredients and free of pure water as a carrier.

11. The anaplerotic Olebiome composition of claim 1, further comprising at least one SPF agent, and, optionally, at least one excipient and/or formulation stabilizer complex, and wherein the excipient is a single- or a multiple-component excipient-stabilizer complex selected from ‘green” amphiphilic surfactant, biopolymer, or any combination thereof.

12. The anaplerotic anhydrous Olebiome composition of claim 1, in a form of oil, oil enriched emulsion, microemulsion, spray, soap, balm, foam, cream, oleogel, organogel, waxes blend, and solid stick with wax.

13. A method of enhancing the sun protection factor (SPF) of an SPF agent, comprising combining the SPF agent with an anaplerotic Olebiome composition of claim 1.

14. The method of claim 13, wherein combining the SPF agent with the anaplerotic Olebiome composition comprises applying to a sun exposed area of the skin the SPF agent and an amount of the anaplerotic Olebiome composition effective to enhance the sun protection factor (SPF) of the SPF agent.

15. The method of claim 14, wherein the anaplerotic Olebiome composition and the SPF agent are applied simultaneously or consequently.

16. The method of claim 14, wherein the anaplerotic Olebiome composition is applied as: layering as a serum/primer, coating layer before use of SPF agent, add-mixed with SPF agent, or preformulated with SPF agent.

17. The method of claim 15, wherein the sun protection factor (SPF) of an SPF agent is enhanced by between 15% to 55%.

18. A method of preventing and/or treating symptoms associated with at least one of at least one of
a. aging;
b. cellular oxidative stress;
c. external radiation induced oxidation; or,
d. any combination thereof,
comprising administering to a subject in need an amount of the anaplerotic Olebiome composition of claim 1 effective to prevent and/or treat the symptoms.

19. The method of claim 18, wherein the symptoms are selected from mitochondrial dysfunction, dryness, loss of water, loss of skin vitality, loss of elasticity, wrinkles, inflammation, microbiome imbalance, or any combination thereof.

20. A process for the preparation of the anaplerotic Olebiome composition of claim 1, comprising partially oxidating triolein of the plant carrier oil under anhydrous conditions, without hydrolyzing the acyl glycerol bond and preventing formation of lipid hydroperoxides and volatile by rancidity associated byproducts by adding lipophilic antioxidant.
